(19) United States
(12) Patent Application Publication
(10) Pub. No.: US 2026/0055113 A1
(43) Pub. Date: Feb. 26, 2026

(54) HETEROARYL COMPOUNDS FOR THE TREATMENT OF CANCER

(71) Applicant: Hoffmann-La Roche Inc., Little Falls, NJ (US)

(72) Inventors: Dong DING, Shanghai (CN); Lan LI, Shanghai (CN); Yanhua LIU, Shanghai (CN); Ming LYU, Shanghai (CN); Hongtao XU, Shanghai (CN); Dan ZHAO, Shanghai (CN); Ge ZOU, Shanghai (CN)

(73) Assignee: Hoffmann-La Roche Inc., Little Falls, NJ (US)

(21) Appl. No.: 19/103,440

(22) PCT Filed: Aug. 10, 2023
(86) PCT No.: PCT/EP2023/072123

§ 371 Date: Feb. 12, 2025

(30) Foreign Application Priority Data
Aug. 12, 2022 (WO) PCT/CN2022/112150
Nov. 30, 2022 (WO) PCT/CN2022/135460

(51) Int. Cl. C07D 487/04; A61K 31/513; A61P 35/00; C07D 498/04

(52) U.S. Cl. CPC C07D 487/04; A61K 31/513; A61P 35/00; C07D 498/04

(57) ABSTRACT

The present invention relates to compounds of formula (I), wherein A¹ to A⁷ and W are as described herein, and their pharmaceutically acceptable salt thereof, and compositions including the compounds and methods of using the compounds.

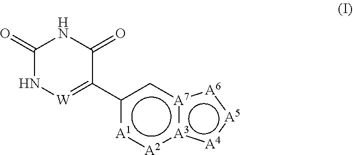

The present invention relates to organic compounds useful for therapy and/or prophylaxis in a mammal, and in particular to inhibition of CD73 useful for treating cancers.

### FIELD OF THE INVENTION

Hyper activation of the adenosine pathway contributes to immunosuppressive tumor microenvironment (TME) that impairs anti-tumor immunity and limits efficacy of immune checkpoint inhibitors. In the last step of the adenosine pathway, the enzyme ecto-5′-nucleotidase (CD73) catalyzes the conversion of AMP to adenosine, which is recognized by the adenosine receptors present in multiple immune cell-types, leading to suppression of the effector T cells and natural killer (NK) cells, activation of the regulatory T (Treg) and myeloid-derived suppressor cells (MDSCs), as well as other changes in the immune system that collectively culminate in an immunosuppressed environment. CD73 is frequently overexpressed in cancers and its upregulation is associated with poor clinical prognosis. Preclinical work in various in vivo tumor models demonstrated restoration of immune cell function and tumor growth inhibition upon genetic ablation or pharmacological inhibition of CD73. Therefore, it is conceivable that alleviating the immunosuppressive TME through CD73 inhibition has the therapeutic potential for restoring anti-tumor immunity and enhancing efficacy of immunotherapy to induce tumor regression. Given the uprising and unmet need for efficacious cancer treatments, inhibition of CD73 activity through administration of small molecule (SM) holds promise. This disclosure describes the invention of new small molecule CD73 inhibitors.

### SUMMARY OF THE INVENTION

The present invention relates to novel compounds of formula (I),

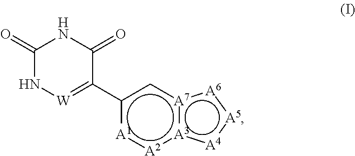

wherein
W is CH or N;
A¹ and A² are each independently CH or N;
A³ and A⁷ are each independently C or N;
A⁴, A⁵ and A⁶ are each independently O, S, N, CR¹ or NR²;
R¹ is H, halogen, cyano, C₁₋₆alkyl, C₃₋₇cycloalkyl, C₁₋₆alkoxyC₁₋₆alkyl or -L¹-R³;
R² is H, C₁₋₆alkyl, C₃₋₇cycloalkyl, C₁₋₆alkoxyC₁₋₆alkyl or -L²-R³;
wherein L¹ is O, S, NH, NR³, C₁₋₆alkylene, C₃₋₇cycloalkylene, heteroarylene or heterocyclylene;
   L² is C₁₋₆alkylene, C₃₋₇cycloalkylene, heteroarylene or heterocyclylene;
   R³ is optionally substituted group selected from C₁₋₆alkyl, C₃₋₇cycloal, C₃₋₇cycloalC₁₋₆alkyl, C₁₋₆alkoxyC₁₋₆alkyl, aryl, heteroaryl, heterocyclyl, arylC₁₋₆alkyl, heterocyclylC₁₋₆alkyl, heteroarylC₁₋₆alkyl, arylhaloC₁₋₆alkyl, heterocyclylhaloC₁₋₆alkyl and heteroarylhaloC₁₋₆alkyl;
or a pharmaceutically acceptable salt thereof.

The compounds of formula (I) show good CD73 inhibition. In another embodiment, the compounds of this invention showed superior cancer cell inhibition. In addition, the compounds of formula (I) also show good or improved human hepatocyte stability, cytotoxicity and solubility profiles.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “C₁₋₆alkoxy” denotes C₁₋₆alkyl-O—.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl or trifluoromethyl.

The term “haloC₁₋₆alkoxy” denotes haloC₁₋₆alkyl-O—.

The term “halophenyl” denotes a phenyl group wherein at least one of the hydrogen atoms of the phenyl group has been replaced by same or different halogen atoms, particularly chloro or fluoro atoms. Examples of halophenyl include chlorophenyl or fluorophenyl.

The term “halopyridinyl” denotes a pyridinyl group wherein at least one of the hydrogen atoms of the pyridinyl group has been replaced by same or different halogen atoms.

The term “halopyridazinyl” denotes a pyridazinyl group wherein at least one of the hydrogen atoms of the pyridazinyl group has been replaced by same or different halogen atoms.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “C₃₋₇cycloalkylene” denotes a divalent C₃₋₇cycloalkyl group.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-11 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Examples of heterocycles include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-onyl, azaspiro[5.5]undecanyl, tetrahydroindolyl, octahydroindolyl, tetrahydroisoindolyl, tetrahydroindazolyl, 1,1-dioxohexahydrothiopyranyl, and 2,3,4a,5,7,7a-hexahydro-[1,4]dioxino[2,3-c]pyrrolyl.

The term “heterocyclylene” denotes a divalent heterocyclyl group.

The term “aryl” denotes a monovalent aromatic carbocyclic mono- or bicyclic ring system comprising 6 to 10 carbon ring atoms. Examples of aryl moieties include phenyl and naphthyl.

The term “arylene” denotes a divalent aryl group.

The term “heteroaryl” refers to any mono-, bi-, or tricyclic aromatic ring system containing from 1 to 4 heteroatoms selected from nitrogen, oxygen, and sulfur, and in an example embodiment, at least one heteroatom is nitrogen. See, for example, Lang's Handbook of Chemistry (Dean, J. A., ed.) 13^{th} ed. Table 7-2 [1985]. Included in the definition are any bicyclic groups where any of the above heteroaryl rings are fused to an aryl ring, wherein the aryl ring or the heteroaryl ring is joined to the remainder of the molecule. In one embodiment, heteroaryl includes 5-6 membered monocyclic aromatic groups where one or more ring atoms is nitrogen, sulfur or oxygen. In one embodiment, heteroaryl includes 7-12 membered bicyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl, 1H-pyrazolo[3,4-d]pyrimidine, 1H-pyrazolo[3,4-d]pyridazine, imidazo[1,5-a]pyrazine, imidazo[5,1-f][1,2,4]triazine, [1,2,4]triazolo[4,3-a]pyrazine, 1H-pyrazolo[3,4-c]pyridazine, 1H-pyrazolo[3,4-b]pyridine, 1H-pyrazolo[4,3-d]pyrimidine, 1H-pyrazolo[3,4-c]pyridine, 1H-pyrazolo[4,3-c]pyridine and purinyl, as well as benzo-fused derivatives, for example benzoxazolyl, benzofuryl, benzothiazolyl, benzothiadiazolyl, benzotriazolyl, benzoimidazolyl, indazolyl and indolyl.

The term “heteroarylene” denotes a divalent heteroaryl group.

In particular embodiments, a heterocyclyl group or a heteroaryl group is attached at a carbon atom of the heterocyclyl group or the heteroaryl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group or heteroaryl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

In one embodiment, the skilled in the art can understand the keto-enol tautomerism may exist for certain structures as illustrated below:

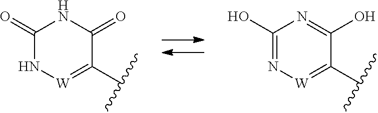

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment an optionally substituted group has 2 substituents. In another embodiment an optionally substituted group has 3 substituents. In another embodiment an optionally substituted group has 4 substituents. In another embodiment an optionally substituted group has 5 substituents.

The term “protecting group” or “PG” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor of CD73

The present invention relates to (i) a compound of formula (I),

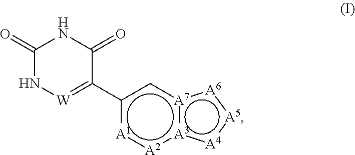

wherein

### W is CH or N;

A¹ and A² are each independently CH or N;
A³ and A⁷ are each independently C or N;
A⁴, A⁵ and A⁶ are each independently O, S, N, CR¹ or NR²;
R¹ is H, halogen, cyano, C₁₋₆alkyl, C₃₋₇cycloalkyl, C₁₋₆alkoxyC₁₋₆alkyl or -L¹-R³;
R² is H, C₁₋₆alkyl, C₃₋₇cycloalkyl, C₁₋₆alkoxyC₁₋₆alkyl or -L²-R³;
wherein L¹ is O, S, NH, NR³, C₁₋₆alkylene, C₃₋₇cycloalkylene, heteroarylene or heterocyclylene;
L² is C₁₋₆alkylene, C₃₋₇cycloalkylene, heteroarylene or heterocyclylene;
R³ is optionally substituted group selected from C₁₋₆alkyl, C₃₋₇cycloal, C₃₋₇cycloalC₁₋₆alkyl, C₁₋₆alkoxyC₁₋₆alkyl, aryl, heteroaryl, heterocyclyl, arylC₁₋₆alkyl, heterocyclylC₁₋₆alkyl, heteroarylC₁₋₆alkyl, arylhaloC₁₋₆alkyl, heterocyclylhaloC₁₋₆alkyl and heteroarylhaloC₁₋₆alkyl;
   or a pharmaceutically acceptable salt thereof.

Another embodiment of present invention is (ii) a compound of formula (Ia) according to (i),

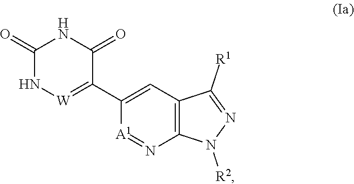

wherein
W is CH;
A¹ is N;
R¹ is (C₁₋₆alkyl)₂amino, (C₁₋₆alkylhalopyrazolyl)C₁₋₆alkoxy, (C₁₋₆alkylhalopyridinyl)C₁₋₆alkoxy, (C₁₋₆alkylpyrazolyl)C₁₋₆alkoxy, (C₁₋₆alkylpyridinyl)C₁₋₆alkoxy, (C₁₋₆alkylpyridinyl)haloC₁₋₆alkoxy, (C₁₋₆alkylthiazolyl)C₁₋₆alkoxy, (cyanophenyl)C₁₋₆alkoxy, (haloC₁₋₆alkylphenyl)C₁₋₆alkoxy, (halophenyl)C₁₋₆alkoxy, (halopyridazinyl)C₁₋₆alkoxy, (halopyridinyl)C₁₋₆alkoxy, (halopyridinyl)haloC₁₋₆alkoxy, (phenylC₁₋₆alkyl)pyrazolyl, benzoxazolylC₁₋₆alkoxy, C₁₋₆alkoxy, C₁₋₆alkyl, C₃₋₇cycloalkyl, C₃₋₇cycloalkyl(C₁₋₆alkyl)amino, C₃₋₇cycloalkylC₁₋₆alkyl(C₁₋₆alkyl)amino, phenylC₁₋₆alkoxy, phenylC₁₋₆alkyl, phenylC₁₋₆alkyl(C₁₋₆alkyl)amino, phenylC₁₋₆alkylamino, phenylC₃₋₇cycloalkyl, phenylhaloC₁₋₆alkoxy, pyridinylC₁₋₆alkoxy or pyridinylhaloC₁₋₆alkoxy;
R² is C₁₋₆alkyl;
or a pharmaceutically acceptable salt thereof.

A further embodiment of present invention is (iii) a compound of formula (Ia) according to (i) or (ii), or a pharmaceutically acceptable salt thereof, wherein R¹ is (C₁₋₆alkylpyridinyl)haloC₁₋₆alkoxy, (halopyridinyl)haloC₁₋₆alkoxy, (phenylC₁₋₆alkyl)pyrazolyl, C₃₋₇cycloalkyl, C₃₋₇cycloalkylC₁₋₆alkyl(C₁₋₆alkyl)amino, phenylC₁₋₆alkoxy, phenylC₁₋₆alkyl, phenylC₁₋₆alkyl(C₁₋₆alkyl)amino, phenylC₁₋₆alkylamino, phenylC₃₋₇cycloalkyl, phenylhaloC₁₋₆alkoxy, pyridinylC₁₋₆alkoxy or pyridinylhaloC₁₋₆alkoxy.

A further embodiment of present invention is (iv) a compound of formula (Ia) according to any one of (i) to (iii), or a pharmaceutically acceptable salt thereof, wherein R¹ is (1-phenylethyl)amino, 1-(2-pyridinyl)ethoxy, 1-(2-pyridinyl)ethoxy, 1-cyclopentylethyl(methyl)amino, 1-phenylcyclopropyl, 1-phenylethoxy, 1-phenylethoxy, 1-phenylethyl, 2,2,2-trifluoro-1-(2-pyridinyl)ethoxy, 2,2,2-trifluoro-1-phenyl-ethoxy, 2,2-difluoro-1-(2-pyridinyl)ethoxy, 2,2-difluoro-1-(5-fluoro-2-pyridinyl)ethoxy, 2,2-difluoro-1-(6-methyl-2-pyridinyl)ethoxy, 2,2-difluoro-1-phenyl-ethoxy, 2-benzylpyrazol-3-yl, cyclobutyl or methyl(1-phenylethyl)amino.

A further embodiment of present invention is (v) a compound of formula (Ia) according to any one of (i) to (iv), or a pharmaceutically acceptable salt thereof, wherein R¹ is (C₁₋₆alkylpyridinyl)haloC₁₋₆alkoxy, (halopyridinyl)haloC₁₋₆alkoxy, C₃₋₇cycloalkylC₁₋₆alkyl(C₁₋₆alkyl)amino, phenylC₁₋₆alkoxy, phenylC₁₋₆alkyl(C₁₋₆alkyl)amino, phenylC₁₋₆alkyl, phenylC₁₋₆alkylamino, phenylhaloC₁₋₆alkoxy, pyridinylC₁₋₆alkoxy or pyridinylhaloC₁₋₆alkoxy.

A further embodiment of present invention is (vi) a compound of formula (Ia) according to any one of (i) to (v), or a pharmaceutically acceptable salt thereof, wherein R¹ is (1-phenylethyl)amino, 1-(2-pyridinyl)ethoxy, 1-cyclopentylethyl(methyl)amino, 1-phenylethoxy, 1-phenylethyl, 2,2-difluoro-1-(2-pyridinyl)ethoxy, 2,2-difluoro-1-(5-fluoro-2-pyridinyl)ethoxy, 2,2-difluoro-1-(6-methyl-2-pyridinyl)ethoxy, 2,2-difluoro-1-phenyl-ethoxy or methyl(1-phenylethyl)amino.

A further embodiment of present invention is (vii) a compound of formula (I) according to any one of (i) to (vi), wherein R² is methyl.

A further embodiment of present invention is (viii) a compound of formula (Ia) or a pharmaceutically acceptable salt thereof, according to any one of (i) to (v), wherein
W is CH;
A¹ is N;
R¹ is (C₁₋₆alkylpyridinyl)haloC₁₋₆alkoxy, (halopyridinyl)haloC₁₋₆alkoxy, C₃₋₇cycloalkylC₁₋₆alkyl(C₁₋₆alkyl)amino, phenylC₁₋₆alkoxy, phenylC₁₋₆alkyl(C₁₋₆alkyl)amino, phenylC₁₋₆alkyl, phenylC₁₋₆alkylamino, phenylhaloC₁₋₆alkoxy, pyridinylC₁₋₆alkoxy or pyridinylhaloC₁₋₆alkoxy;
R² is C₁₋₆alkyl;
or a pharmaceutically acceptable salt thereof.

A further embodiment of present invention is (ix) a compound of formula (Ia) or a pharmaceutically acceptable salt thereof, according to any one of (i) to (vi), wherein
W is CH;
A¹ is N;
R¹ is (1-phenylethyl)amino, 1-(2-pyridinyl)ethoxy, 1-cyclopentylethyl(methyl)amino, 1-phenylethoxy, 1-phenylethyl, 2,2-difluoro-1-(2-pyridinyl)ethoxy, 2,2-difluoro-1-(5-fluoro-2-pyridinyl)ethoxy, 2,2-difluoro-1-(6-methyl-2-pyridinyl)ethoxy, 2,2-difluoro-1-phenyl-ethoxy or methyl(1-phenylethyl)amino;
R² is methyl;
or a pharmaceutically acceptable salt thereof.

Another embodiment of present invention is (x) a compound of formula (Ib) according to (i),

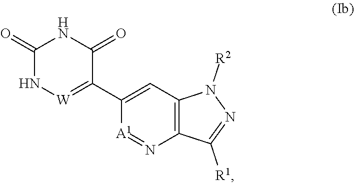

wherein
W is CH;
A¹ is N;
R¹ is H or halogen;
R² is C₁₋₆alkyl;
or a pharmaceutically acceptable salt thereof.

A further embodiment of present invention is (xi) a compound of formula (Ib) according to (x), wherein R¹ is halogen.

A further embodiment of present invention is (xii) a compound of formula (Ib) according to (x) or (xi), wherein R¹ is chloro.

A further embodiment of present invention is (xiii) a compound of formula (Ib) according to any one of (x) to (xii), wherein R² is methyl.

A further embodiment of present invention is (xiv) a compound of formula (Ib) according to any one of (x) to (xiii), wherein
W is CH;
A¹ is N;
R¹ is chloro;
R² is methyl;
or a pharmaceutically acceptable salt thereof.

Another embodiment of present invention is (xv) a compound of formula (Ic) according to (i),

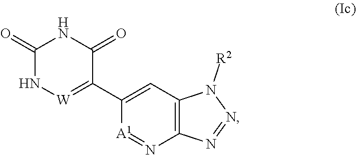

wherein
W is CH;
A¹ is N;
R² is C₁₋₆alkyl;
or a pharmaceutically acceptable salt thereof.

A further embodiment of present invention is (xvi) a compound of formula (Ic) according to (xv), wherein R² is methyl.

Another embodiment of present invention is (xvii) a compound of formula (Id) according to (i),

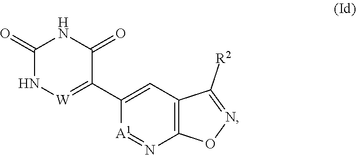

wherein
W is CH;
A¹ is N;
R¹ is C₃₋₇cycloalkyl;
or a pharmaceutically acceptable salt thereof.

A further embodiment of present invention is (xviii) a compound of formula (Id) according to (xvii), wherein R¹ is cyclobutyl.

The present invention relates to (i′) a compound of formula (I),

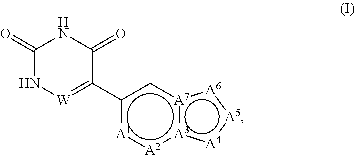

wherein
W is CH or N;
A¹ and A² are each independently CH or N;
A³ and A⁷ are each independently C or N;
A⁴, A⁵ and A⁶ are each independently N, CR¹ or NR²;
R¹ is H, halogen, cyano, C₁₋₆alkyl, C₃₋₇cycloalkyl, C₁₋₆alkoxyC₁₋₆alkyl or -L¹-R³;
R² is H, C₁₋₆alkyl, C₃₋₇cycloalkyl, C₁₋₆alkoxyC₁₋₆alkyl or -L²-R³;
wherein L¹ is O, S, NH, C₁₋₆alkylene, C₃₋₇cycloalkylene, heteroarylene or heterocyclylene;
   L² is C₁₋₆alkylene, C₃₋₇cycloalkylene, heteroarylene or heterocyclylene;
   R³ is optionally substituted aryl, heteroaryl, heterocyclyl, arylC₁₋₆alkyl, heterocyclylC₁₋₆alkyl or heteroarylC₁₋₆alkyl;
or a pharmaceutically acceptable salt thereof.

Another embodiment of present invention is (ii′) a compound of formula (Ia) according to (i′),

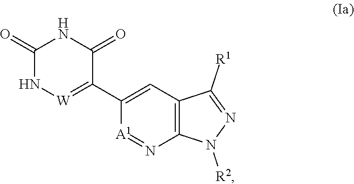

wherein
W is CH;
A¹ is N;
R¹ is (cyanophenyl)C₁₋₆alkoxy, (haloC₁₋₆alkylphenyl)C₁₋₆alkoxy, (halophenyl)C₁₋₆alkoxy, (phenylC₁₋₆alkyl)pyrazolyl, C₁₋₆alkoxy, C₁₋₆alkyl, C₃₋₇cycloalkyl, phenylC₁₋₆alkoxy, phenylC₁₋₆alkyl, phenylC₃₋₇cycloalkyl, pyridinylC₁₋₆alkoxy;
R² is C₁₋₆alkyl;
or a pharmaceutically acceptable salt thereof.

A further embodiment of present invention is (iii′) a compound of formula (Ia) according to (i′) or (ii′), or a pharmaceutically acceptable salt thereof, wherein R¹ is (phenylC₁₋₆alkyl)pyrazolyl, phenylC₁₋₆alkoxy, phenylC₁₋₆alkyl, phenylC₃₋₇cycloalkyl or pyridinylC₁₋₆alkoxy.

A further embodiment of present invention is (iv′) a compound of formula (Ia) according to any one of (i′) to (iii′), or a pharmaceutically acceptable salt thereof, wherein R¹ is 1-(2-pyridinyl)ethoxy, 1-phenylcyclopropyl, 1-phenylethoxy, 1-phenylethyl or 2-benzylpyrazol-3-yl.

A further embodiment of present invention is (v′) a compound of formula (I) according to any one of (i′) to (iv′), wherein R² is methyl.

A further embodiment of present invention is (vi′) a compound of formula (Ia) or a pharmaceutically acceptable salt thereof, according to any one of (i′) to (v′), wherein
W is CH;
A¹ is N;
R¹ is (phenylC₁₋₆alkyl)pyrazolyl, phenylC₁₋₆alkoxy, phenylC₁₋₆alkyl, phenylC₃₋₇cycloalkyl or pyridinylC₁₋₆alkoxy;
R² is C₁₋₆alkyl;
or a pharmaceutically acceptable salt thereof.

A further embodiment of present invention is (vii′) a compound of formula (Ia) or a pharmaceutically acceptable salt thereof, according to any one of (i′) to (vi′), wherein
W is CH;
A¹ is N;
R¹ is 1-(2-pyridinyl)ethoxy, 1-phenylcyclopropyl, 1-phenylethoxy, 1-phenylethyl or 2-benzylpyrazol-3-yl;
R² is methyl;
or a pharmaceutically acceptable salt thereof.

Another embodiment of present invention is (viii′) a compound of formula (Ib) according to (i′),

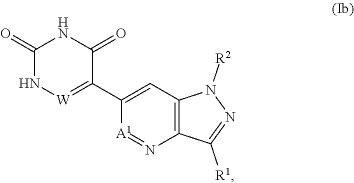

wherein
W is CH;
A¹ is N;
R¹ is H or halogen;
R² is C₁₋₆alkyl;
or a pharmaceutically acceptable salt thereof.

A further embodiment of present invention is (ix′) a compound of formula (Ib) according to (viii′), wherein R¹ is halogen.

A further embodiment of present invention is (x′) a compound of formula (Ib) according to (viii′) or (ix′), wherein R¹ is chloro.

A further embodiment of present invention is (xi′) a compound of formula (Ib) according to any one of (viii′) to (x′), wherein R² is methyl.

A further embodiment of present invention is (xii′) a compound of formula (Ib) according to any one of (viii′) to (xi′), wherein
W is CH;
A¹ is N;
R¹ is chloro;
R² is methyl;
or a pharmaceutically acceptable salt thereof.

Another embodiment of present invention is (xiii′) a compound of formula (Ic) according to (i′),

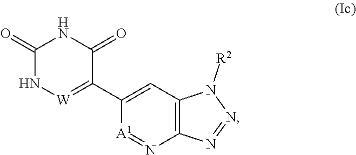

wherein
W is CH;
A¹ is N;
R² is C₁₋₆alkyl;
or a pharmaceutically acceptable salt thereof.

A further embodiment of present invention is (xiv′) a compound of formula (Ic) according to (xiii′), wherein R² is methyl.

Another embodiment of present invention is (xix) a compound selected from the following:
5-(1,3-dimethylpyrazolo[3,4-c]pyridazin-5-yl)-1H-pyrimidine-2,4-dione;
5-(3-cyclopropyl-1-methyl-pyrazolo[3,4-c]pyridazin-5-yl)-1H-pyrimidine-2,4-dione;
5-(1-methylpyrazolo[4,3-c]pyridazin-6-yl)-1H-pyrimidine-2,4-dione;
5-(1-methyltriazolo[4,5-c]pyridazin-6-yl)-1H-pyrimidine-2,4-dione;
5-[1-methyl-3-(1-phenylethyl)pyrazolo[3,4-c]pyridazin-5-yl]-1H-pyrimidine-2,4-dione;
5-[1-methyl-3-(1-phenylcyclopropyl)pyrazolo[3,4-c]pyridazin-5-yl]-1H-pyrimidine-2,4-dione;
5-[3-(2-benzylpyrazol-3-yl)-1-methyl-pyrazolo[3,4-c]pyridazin-5-yl]-1H-pyrimidine-2,4-dione;
5-(3-isopropoxy-1-methyl-pyrazolo[3,4-c]pyridazin-5-yl)-1H-pyrimidine-2,4-dione;
5-(3-chloro-1-methyl-pyrazolo[4,3-c]pyridazin-6-yl)-1H-pyrimidine-2,4-dione;
5-[1-methyl-3-(1-phenylethoxy)pyrazolo[3,4-c]pyridazin-5-yl]-1H-pyrimidine-2,4-dione;
3-[1-[5-(2,4-dioxo-1H-pyrimidin-5-yl)-1-methyl-pyrazolo[3,4-c]pyridazin-3-yl]oxyethyl]benzonitrile;
4-[1-[5-(2,4-dioxo-1H-pyrimidin-5-yl)-1-methyl-pyrazolo[3,4-c]pyridazin-3-yl]oxyethyl]benzonitrile;
5-[3-[1-(2-chlorophenyl)ethoxy]-1-methyl-pyrazolo[3,4-c]pyridazin-5-yl]-1H-pyrimidine-2,4-dione;
5-[3-[1-(3-chlorophenyl)ethoxy]-1-methyl-pyrazolo[3,4-c]pyridazin-5-yl]-1H-pyrimidine-2,4-dione;
5-[3-[1-(4-chlorophenyl)ethoxy]-1-methyl-pyrazolo[3,4-c]pyridazin-5-yl]-1H-pyrimidine-2,4-dione;
5-[3-[1-(4-fluorophenyl)ethoxy]-1-methyl-pyrazolo[3,4-c]pyridazin-5-yl]-1H-pyrimidine-2,4-dione;
5-[1-methyl-3-[1-(2-pyridyl)ethoxy]pyrazolo[3,4-c]pyridazin-5-yl]-1H-pyrimidine-2,4-dione;
5-[1-methyl-3-[1-(3-pyridyl)ethoxy]pyrazolo[3,4-c]pyridazin-5-yl]-1H-pyrimidine-2,4-dione;
5-[1-methyl-3-[1-(4-pyridyl)ethoxy]pyrazolo[3,4-c]pyridazin-5-yl]-1H-pyrimidine-2,4-dione;
5-[1-methyl-3-[1-[3-(trifluoromethyl)phenyl]ethoxy]pyrazolo[3,4-c]pyridazin-5-yl]-1H-pyrimidine-2,4-dione;
5-[1-methyl-3-[1-(2-methylthiazol-4-yl)ethoxy]pyrazolo[3,4-c]pyridazin-5-yl]-1H-pyrimidine-2,4-dione;
5-[1-methyl-3-[1-(5-methylthiazol-2-yl)ethoxy]pyrazolo[3,4-c]pyridazin-5-yl]-1H-pyrimidine-2,4-dione;
5-[3-[1-(4-chloro-1-methyl-pyrazol-3-yl)ethoxy]-1-methyl-pyrazolo[3,4-c]pyridazin-5-yl]-1H-pyrimidine-2,4-dione;
5-[1-methyl-3-[1-(2-methylpyrazol-3-yl)ethoxy]pyrazolo[3,4-c]pyridazin-5-yl]-1H-pyrimidine-2,4-dione;
5-[3-[1-(1,3-benzoxazol-2-yl)ethoxy]-1-methyl-pyrazolo[3,4-c]pyridazin-5-yl]-1H-pyrimidine-2,4-dione;
5-[1-methyl-3-[(1S)-1-(2-pyridyl)ethoxy]pyrazolo[3,4-c]pyridazin-5-yl]-1H-pyrimidine-2,4-dione;
5-[1-methyl-3-[(1S)-1-phenylethoxy]pyrazolo[3,4-c]pyridazin-5-yl]-1H-pyrimidine-2,4-dione;
5-[3-[(1R)-2,2-difluoro-1-phenyl-ethoxy]-1-methyl-pyrazolo[3,4-c]pyridazin-5-yl]-1H-pyrimidine-2,4-dione;
5-[1-methyl-3-[(1R)-2,2,2-trifluoro-1-(2-pyridyl)ethoxy]pyrazolo[3,4-c]pyridazin-5-yl]-1H-pyrimidine-2,4-dione;
5-[1-methyl-3-[(1S)-2,2,2-trifluoro-1-(2-pyridyl)ethoxy]pyrazolo[3,4-c]pyridazin-5-yl]-1H-pyrimidine-2,4-dione;
5-[1-methyl-3-[(1R)-2,2,2-trifluoro-1-phenyl-ethoxy]pyrazolo[3,4-c]pyridazin-5-yl]-1H-pyrimidine-2,4-dione;
5-[1-methyl-3-[(1S)-2,2,2-trifluoro-1-phenyl-ethoxy]pyrazolo[3,4-c]pyridazin-5-yl]-1H-pyrimidine-2,4-dione;
5-[3-[(1R)-2,2-difluoro-1-(2-pyridyl)ethoxy]-1-methyl-pyrazolo[3,4-c]pyridazin-5-yl]-1H-pyrimidine-2,4-dione;
5-[3-[(1S)-2,2-difluoro-1-(2-pyridyl)ethoxy]-1-methyl-pyrazolo[3,4-c]pyridazin-5-yl]-1H-pyrimidine-2,4-dione;
5-[3-1[(1R)-2,2-difluoro-1-(5-fluoro-2-pyridyl)ethoxy]-1-methyl-pyrazolo[3,4-c]pyridazin-5-yl]-1H-pyrimidine-2,4-dione;
5-[3-[(1S)-2,2-difluoro-1-(5-fluoro-2-pyridyl)ethoxy]-1-methyl-pyrazolo[3,4-c]pyridazin-5-yl]-1H-pyrimidine-2,4-dione;
5-[3-[(1R)-2,2-difluoro-1-(6-methyl-2-pyridyl)ethoxy]-1-methyl-pyrazolo[3,4-c]pyridazin-5-yl]-1H-pyrimidine-2,4-dione;
5-[3-[(1S)-2,2-difluoro-1-(6-methyl-2-pyridyl)ethoxy]-1-methyl-pyrazolo[3,4-c]pyridazin-5-yl]-1H-pyrimidine-2,4-dione;
5-[1-methyl-3-[methyl(1,2,2-trimethylpropyl)amino]pyrazolo[3,4-c]pyridazin-5-yl]-1H-pyrimidine-2,4-dione;
5-[3-[1-cyclopentylethyl(methyl)amino]-1-methyl-pyrazolo[3,4-c]pyridazin-5-yl]-1H-pyrimidine-2,4-dione;
5-[3-[cyclopentyl(methyl)amino]-1-methyl-pyrazolo[3,4-c]pyridazin-5-yl]-1H-pyrimidine-2,4-dione;
5-[1-methyl-3-[[(1S)-1-phenylethyl]amino]pyrazolo[3,4-c]pyridazin-5-yl]-1H-pyrimidine-2,4-dione;
5-[1-methyl-3-[methyl-[(1S)-1-phenylethyl]amino]pyrazolo[3,4-c]pyridazin-5-yl]-1H-pyrimidine-2,4-dione;
5-(3-cyclobutyl-1-methyl-pyrazolo[3,4-c]pyridazin-5-yl)-1H-pyrimidine-2,4-dione;
5-[3-[(1S)-1-(5-fluoro-6-methyl-2-pyridyl)ethoxy]-1-methyl-pyrazolo[3,4-c]pyridazin-5-yl]-1H-pyrimidine-2,4-dione;
5-[1-methyl-3-[(1S)-1-(6-methyl-2-pyridyl)ethoxy]pyrazolo[3,4-c]pyridazin-5-yl]-1H-pyrimidine-2,4-dione;
5-[1-methyl-3-[(1S)-1-(4-methyl-2-pyridyl)ethoxy]pyrazolo[3,4-c]pyridazin-5-yl]-1H-pyrimidine-2,4-dione;
5-[3-[(S)-1-(5-fluoro-2-pyridyl)ethoxy]-1-methyl-pyrazolo[3,4-c]pyridazin-5-yl]-1H-pyrimidine-2,4-dione;
5-[3-[(1S)-1-(6-chloro-2-pyridyl)ethoxy]-1-methyl-pyrazolo[3,4-c]pyridazin-5-yl]-1H-pyrimidine-2,4-dione;
5-[3-[(1S)-1-(6-chloropyridazin-3-yl)ethoxy]-1-methyl-pyrazolo[3,4-c]pyridazin-5-yl]-1H-pyrimidine-2,4-dione; and
5-(3-cyclobutylisoxazolo[5,4-c]pyridazin-5-yl)-1H-pyrimidine-2,4-dione;
   or a pharmaceutically acceptable salt thereof.

Another embodiment of present invention is related to (xx) a process for the preparation of a compound according to any one of (i) to (xv) comprising any one of the following steps:
a) Deprotection of compound of formula (XVI),

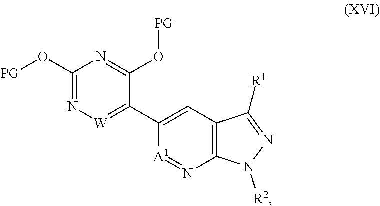

with an acid or dealkylation reagent, or through hydrogenation to afford the compound of formula (Ia),

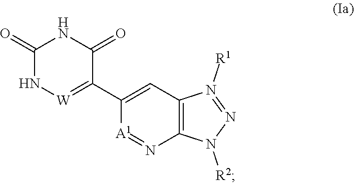

b) Deprotection of compound of formula (XXI),

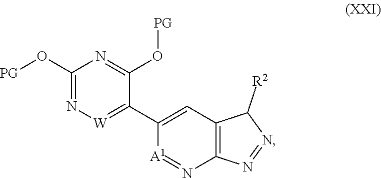

with an acid or dealkylation reagent, or through hydrogenation to afford the compound of formula (Ib-1),

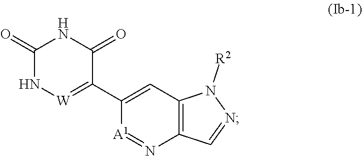

c) Deprotection of compound of formula (XXVI),

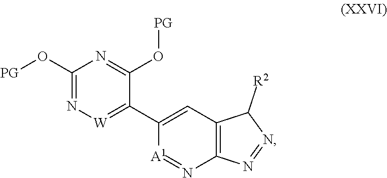

with an acid or dealkylation reagent, or through hydrogenation to afford the compound of formula (Ic),

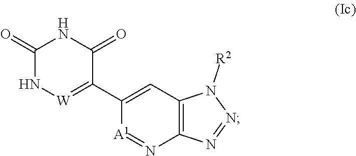

d) Deprotection of compound of formula (XXXII),

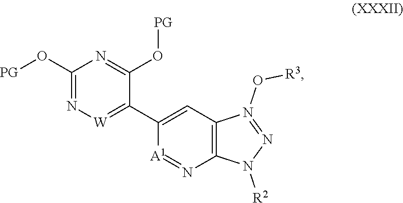

with an acid or dealkylation reagent, or through hydrogenation to afford the compound of formula (Ia-1),

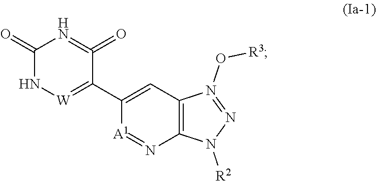

e) Deprotection of compound of formula (XXXVI),

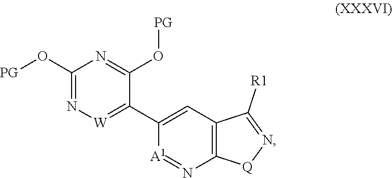

with an acid or dealkylation reagent, or through hydrogenation to afford the compound of formula (XXXVII),

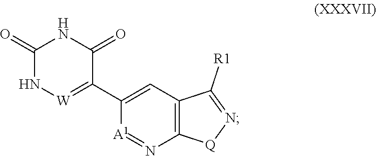

wherein
each PG is independently an oxygen protecting group; wherein PG is selected from methyl, tert-butyl, TBS, ethoxymethyl and benzyl;
in step a), b), c), d) and e), the acid is trifluoroacetic acid or aqueous hydrochloric acid; the dealkylation reagent is TMSCl and NaI; the hydrogenation is conducted with Pd/C;
A¹, W, R¹ to R³ are defined as in any one of claims (i) to (xviii) or (i′) to (xiv′).

Another embodiment of present invention is (xxi) a compound or pharmaceutically acceptable salt according to any one of (i) to (xix) or (i′) to (xiv′) for use as therapeutically active substance.

Another embodiment of present invention is (xxii) a pharmaceutical composition comprising a compound in accordance with any one of (i) to (xix) or (i′) to (xiv′) and a pharmaceutically acceptable excipient.

Another embodiment of present invention is (xxiii) the use of a compound according to any one of (i) to (xix) or (i′) to (xiv′) for treating cancers.

Another embodiment of present invention is (xxiv) the use according to (xxiii), wherein the cancer is pancreatic cancer, colorectal cancer, gastric cancer, esophageal cancer, liver cancer, lung cancer, breast cancer, ovarian cancer, prostate cancer or melanoma.

Another embodiment of present invention is (xxv) the use of a compound according to any one of (i) to (xix) or (i′) to (xiv′) for inhibiting CD73.

Another embodiment of present invention is (xxvi) the use of a compound according to any one of (i) to (xix) or (i′) to (xiv′) for the preparation of a medicament for the treatment or prophylaxis of cancers, wherein the cancer is pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia.

Another embodiment of present invention is (xxvii) the use of a compound according to any one of (i) to (xix) or (i′) to (xiv′) for the preparation of a medicament as a CD73 inhibitor.

Another embodiment of present invention is (xxviii) a compound or pharmaceutically acceptable salt according to any one of (i) to (xix) or (i′) to (xiv′), when manufactured according to a process of (xx).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit the enzymatic activity of CD73 protein in converting AMP to adenosine. In one example, the pharmaceutically effective amount of the compound of the invention administered parenterally per dose will be in the range of about 0.01 to 100 mg/kg, alternatively about 0.1 to 50 mg/kg of patient body weight per day, with the typical initial range of compound used being 0.3 to 30 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 0.1 mg to 500 mg of the compound of the invention compounded with about 0.1 to 500 mg anhydrous lactose, about 0.1 to 500 mg sodium croscarmellose, about 0.1 to 500 mg polyvinylpyrrolidone (PVP) K30, and about 0.1 to 500 mg magnesium stearate. The powdered ingredients are first mixed together and then mixed with a solution of the PVP. The resulting composition can be dried, granulated, mixed with the magnesium stearate and compressed to tablet form using conventional equipment.

An example of an aerosol formulation can be prepared by dissolving the compound, for example 1 to 450 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of cancers. Another embodiment includes a pharmaceutical composition comprising a compound of formula (I) for use in the treatment of cancer.

The following embodiments illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention inhibit the enzymatic activity of CD73 in converting AMP to adenosine. Accordingly, the compounds of the invention are useful for reducing the adenosine levels in the TME. Compounds of the invention are useful for promoting immune-mediated killing of cancer cells that overexpress CD73, e.g. pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia. Alternatively, compounds of the invention are useful for promoting immune-mediated killing of cancer cells that are dependent on the adenosine pathway or in malignant solid tumor where the adenosine pathway is potentiated by dysregulation or mutation of effector pathways as EGFR-RAS-MAPK, PI3K-AKT-driven signaling, for targeted therapy in pancreatic adenocarcinoma, non-small cell lung cancer, esophageal and gastric adenocarcinoma, etc. More broadly, the compounds can be used for the treatment and prophylaxis of all cancer types which exhibit immunosuppressive TME.

Another embodiment includes a method of treating or preventing cancer in a mammal in need of such treatment, wherein the method comprises administering to said mammal a therapeutically effective amount of a compound of formula (I), a stereoisomer, tautomer or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R³, and W are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

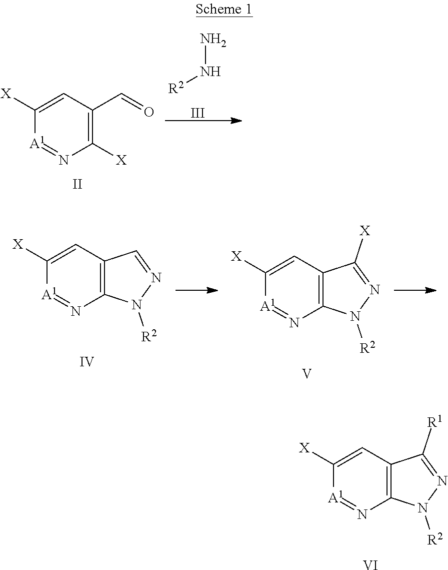

wherein each X is independently halogen.

Compound of formula (VI) can be prepared according to Scheme 1. The di-halogenated heteroaryl aldehyde (II) can be cyclized with hydrazine derivatives (III) to afford compound of formula (IV). Treatment compound of formula (IV) with various halogenation reagents (e.g. NBS, liquid Br₂ or NIS etc.) can provide compound of formula (V). Selective metal catalyzed coupling reaction (such as Buchwald-Hartwig amination, or Ullmann coupling) of compound of formula (V) with halide, R³OH, R³NH₂ or (R³)₂NH, or Chan-Lam coupling between compound of formula (XI) and boronic acid R¹B(OH)₂, or Suzuki-Miyaura type coupling between compound of formula (XI) and boronic acid R¹B(OH)₂ or R¹Bpin can afford compound of formula (VI).

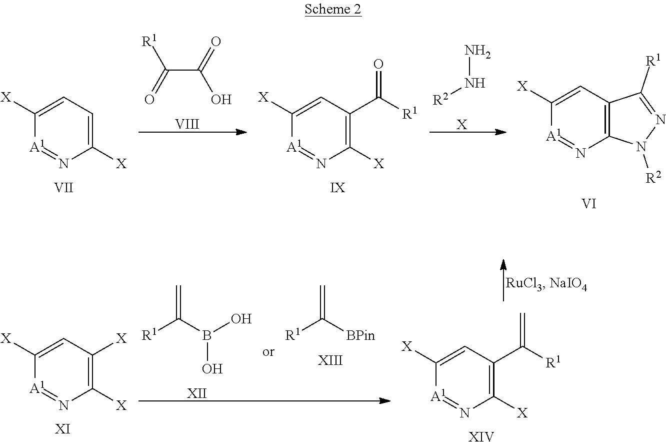

Alternatively, compound of formula (VI) can be prepared according to Scheme 2. A decarboxylative oxidative acylation between compound of formula (VII) and compound of formula (VIII) can be achieved using transition metal (e.g. AgNO₃) as catalyst and persulphate (e.g. Na₂S₂O₈, (NH₄)₂S₂O₈) as oxidant under acidic condition for preparing compound of formula (IX). Treating compound of formula (IX) with compound of formula (X) can achieve the cyclization reaction providing compound of formula (VI).

Compound of formula (IX) can be prepared according to another synthetic route from compound of formula (XI). A regioselective Suzuki-Miyaura type coupling of tri-halogenated heteroarenes (XI) with boronic acid (XII) or compound of formula (XIII) (e.g. boronate ester, trifluoroborate salt) in the presence of a palladium catalyst (e.g. Pd(dppf)Cl₂, Pd(PPh₃)₄, cataCXium-A-Pd-G3, etc.) and bases (e.g. Na₂CO₃, K₂CO₃, Cs₂CO₃, etc.) can be conducted to provide compound of formula (XIV). With the treatment of a catalyst (e.g. RuCl₃, OsO₄ etc.) in combination with an oxidant (e.g. NaIO₄, Oxone™, NaOCl etc.), the alkene group of compound of formula (XIV) can be oxidatively cleaved to a carbonyl group to afford compound of formula (IX).

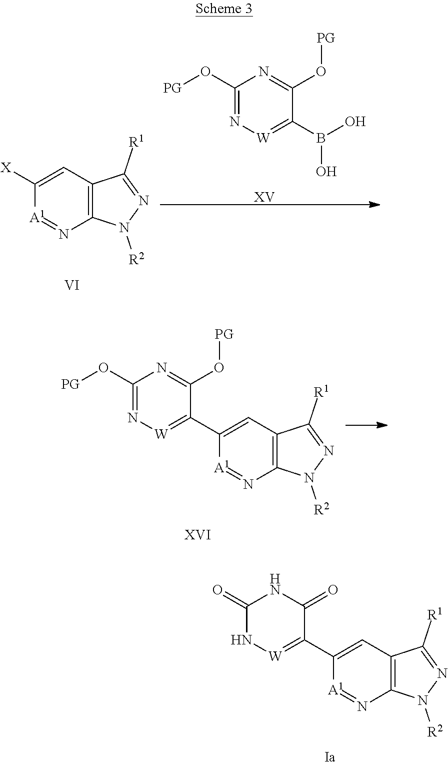

wherein each PG is independently an oxygen protecting group, such as methyl, tert-butyl, TBS, ethoxymethyl and benzyl.

Compound of formula (Ia) can be prepared according to Scheme 3. A Suzuki-Miyaura type coupling of compound of formula (VI) with heteroaryl boronic acid (XV) in the presence of a palladium catalyst (e.g. Pd(dppf)Cl₂, Pd(PPh₃)₄, cataCXium-A-Pd-G3 etc.) and bases (e.g. Na₂CO₃, K₂CO₃, Cs₂CO₃ etc.) can be conducted to provide compound of formula (XVI). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (Ia).

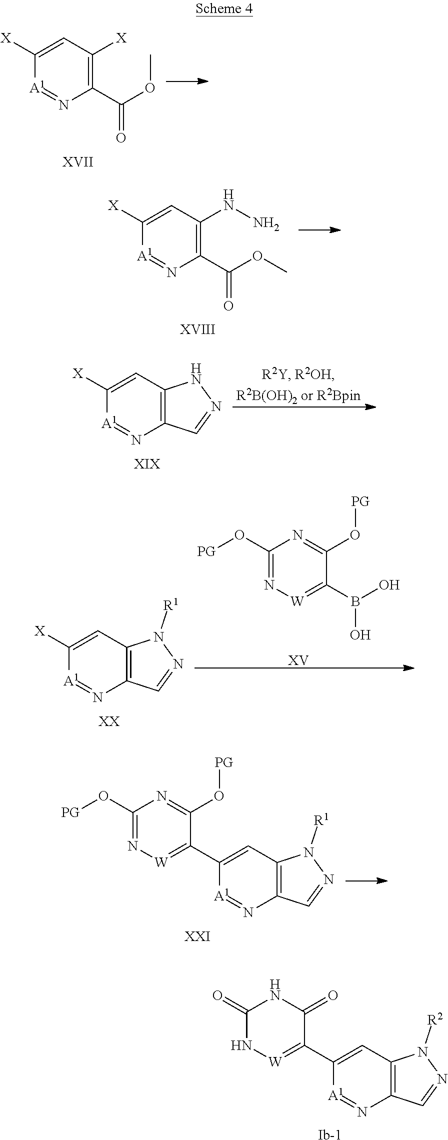

wherein Y is halogen, OTf, OMs, or OTs.

Compound of formula Ib-1) can be prepared according to Scheme 4. A regioselective aromatic nucleophilic substitution reaction (S_{N}Ar) of di-halogenated heteroarenes (XVII) with hydrazine hydrate with or without a non-nucleophilic base can deliver compound of formula (XVIII). A semi-reduction of the methyl ester group of compound of formula (XVIII) to aldehyde with a reducing agent, such as diisobutyl aluminium hydride (DIBAL-H), and then a tandem intramolecular condensation can provide compound of formula (XIX). The defined R² group of compound of formula (XX) can be introduced via nucleophilic substitution of compound of formula (XIX) with R²Y, or via transition metal-mediated coupling reaction (e.g. Buchwald-Hartwig or Ullmann-Ma amination with R²Y, Chan-Lam coupling with R²B(OH)₂ or R²Bpin etc.) or via Mitsunobu reaction with alcohol R²OH. A Suzuki-Miyaura type cross-coupling between compound of formula (XX) and heteroaryl boronic acid (XV) can provide compound of formula (XXI). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (Ib-1).

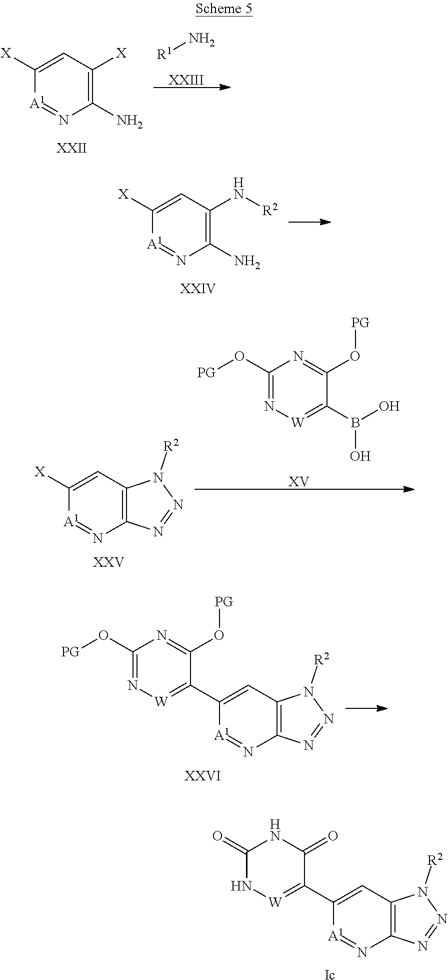

wherein each X is independently halogen.

Compound of formula (Ic) can be prepared according to Scheme 5. A regioselective aromatic nucleophilic substitution reaction (S_{N}Ar) between di-halogenated heteroaryl amine (XXII) and amine (XXIII) in the presence of a non-nucleophilic base, such as N,N-diisopropylethylamine, can deliver compound of formula (XXIV). Treatment of compound of formula (XXIV) with sodium nitrite in an acid condition can provide the triazole (XXV). A Suzuki-Miyaura type cross-coupling between triazole (XXV) and heteroaryl boronic acid (XV) can provide compound of formula (XXVI). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (Ic).

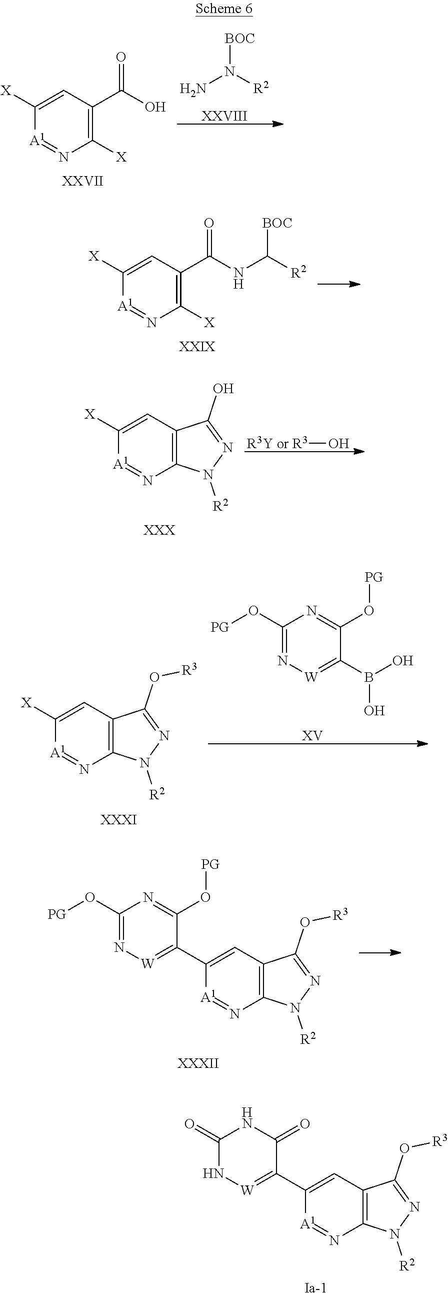

wherein Y is halogen, OTf, OMs, or OTs.

Compound of formula (Ia-1) can be prepared according to Scheme 6. Treatment of acid (XXVII) with oxalyl chloride was followed by reaction with a substituted hydrazine (XXVIII) to afford compound of formula (XXIX). Treatment of compound of formula (XXIX) with an acid, such as TFA or HCl, resulted in intramolecular cyclization to afford the compound of formula (XXX). The compound of formula (XXXI) can be obtained via nucleophilic substitution of compound of formula (XXX) with R³Y or via Mitsunobu reaction with alcohol R³OH. A Suzuki-Miyaura type cross-coupling between compound of formula (XXXI) and heteroaryl boronic acid (XV) can provide compound of formula (XXXII). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (Ia-1).

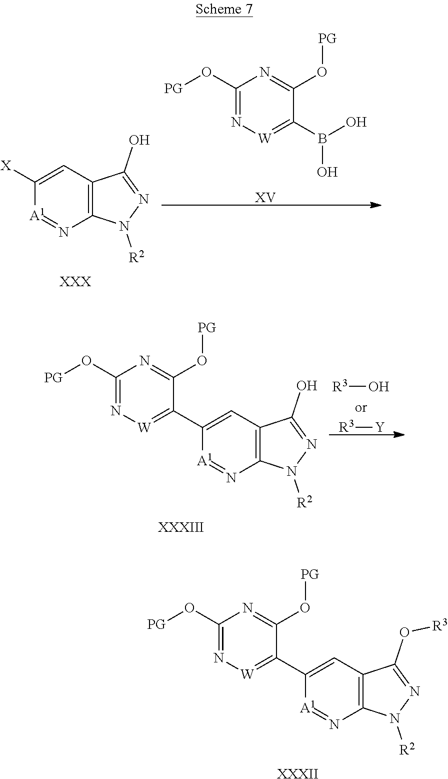

wherein Y is halogen, OTf, OMs, or OTs.

Compound of formula (XXXII) can be alternatively prepared according to Scheme 7. A Suzuki-Miyaura type cross-coupling between compound of formula (XXX) with heteroaryl boronic acid (XV) can provide compound of formula (XXXIII). The compound of formula (XXXII) can be obtained via Mitsunobu reaction of compound of formula (XXXIII) with alcohol R³OH or via nucleophilic substitution with R³Y.

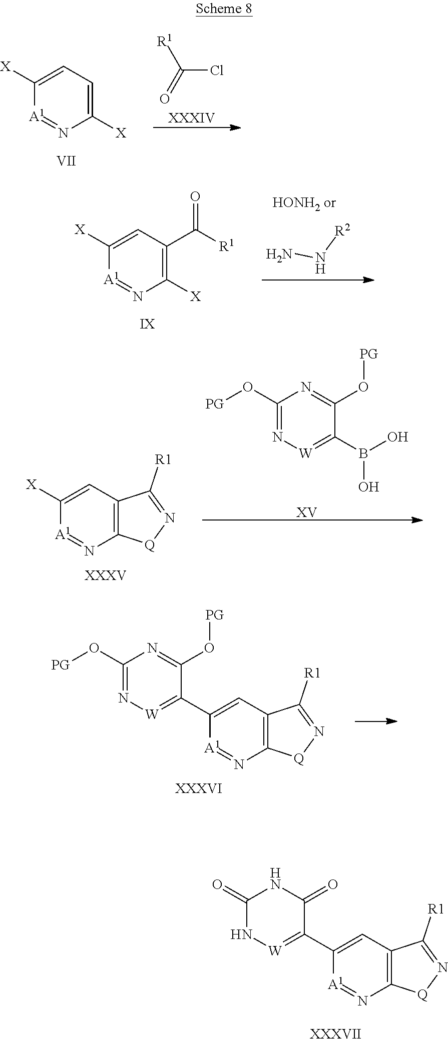

wherein each X is independently halogen. Q is O or NR².

Compound of formula (XXXVII) can be prepared according to Scheme 8. A deprotonative-zincation of compound of formula (VII) using TMPMgCl·LiCl together with ZnCl₂, Zn(OPiv)₂ etc. or using TMPZnCl·LiCl etc. A subsequent acylation reaction of the above organic zinc reagent with the compound of formula (XXXIV) can be achieved with the assistance of transition metal (e.g. CuCN·2LiCl) for preparing compound of formula (IX). Treating compound of formula (IX) with hydroxylamine derivatives or hydrazine derivatives can achieve the cyclization reaction providing compound of formula (XXXV). A Suzuki-Miyaura type cross-coupling between compound of formula (XXXV) and heteroaryl boronic acid (XV) can provide compound of formula (XXXVI). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (XXXVII).

Compounds of this invention can be obtained as mixtures of diastereomers or enantiomers, which can be separated by methods well known in the art, e.g. (chiral) HPLC or SFC. In another embodiment, compounds of this invention can be obtained according to above scheme by using corresponding chiral starting materials.

This invention also relates to a process for the preparation of a compound of formula (I) comprising any of the following steps:
a) Deprotection of compound of formula (XVI),

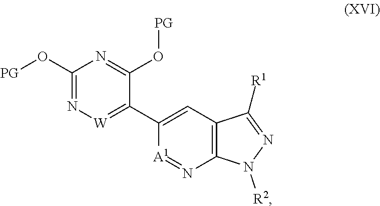

with an acid or dealkylation reagent, or through hydrogenation to afford the compound of formula (Ia),

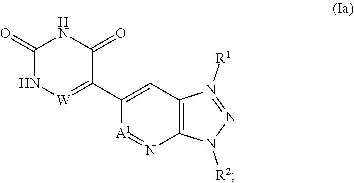

b) Deprotection of compound of formula (XXI),

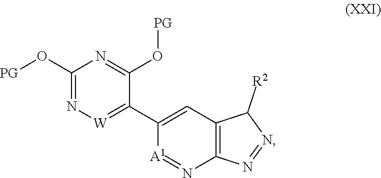

with an acid or dealkylation reagent, or through hydrogenation to afford the compound of formula (Ib-1),

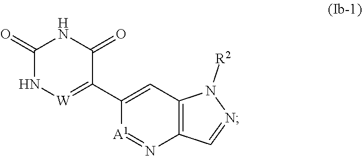

c) Deprotection of compound of formula (XXVI),

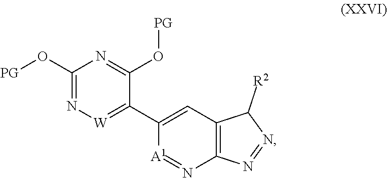

with an acid or dealkylation reagent, or through hydrogenation to afford the compound of formula (Ic),

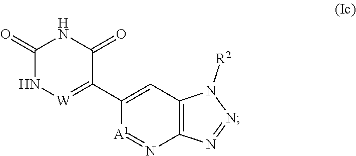

d) Deprotection of compound of formula (XXXII),

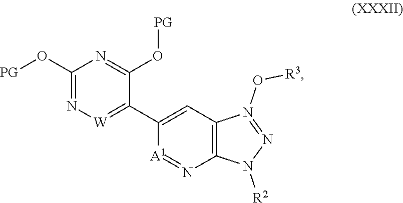

with an acid or dealkylation reagent, or through hydrogenation to afford the compound of formula (Ia-1),

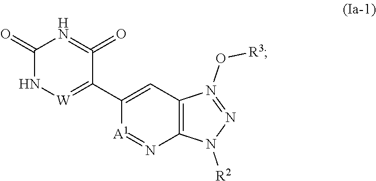

e) Deprotection of compound of formula (XXXVI),

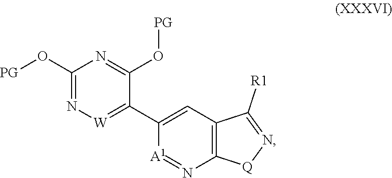

with an acid or dealkylation reagent, or through hydrogenation to afford the compound of formula (XXXVII),

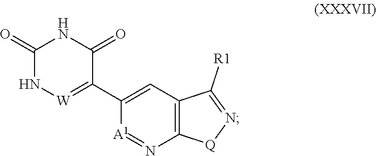

wherein:
in step a), b), c) d) and e), the acid can be, for example, trifluoroacetic acid or aqueous hydrochloric acid; the dealkylation reagent can be, for example TMSCl and NaI; the hydrogenation is conducted with Pd/C.

A compound of formula (I) when manufactured according to the above process is also an object of the invention.

### Examples

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
AcOH: acetic acid
BTMPO N,N′-bis(2,4,6-trimethoxyphenyl)oxamide
DCE: dichloroethane
DCM: dichloromethane
DIPEA or DIEA: N,N-diisopropylethylamine
DIBAL-H: Diisobutylaluminium hydride
DME: Dimethoxyethane
DMF: N,N-Dimethylformamide
DMP Dess-Martin periodinane
EA or EtOAc: ethyl acetate
FA: formic acid
HATU 1-[Bis(dimethylamino)methylene]-1H-1,2,3-triazolo[4,5-b]pyridinium 3-oxide hexafluorophosphate
IC₅₀: half inhibition concentration
LCMS liquid chromatography-mass spectrometry
MS: mass spectrometry
NBS: N-Bromosuccinimide
Pd(dppf)Cl₂·DCM [1,1′-Bis(diphenylphosphino)ferrocene]dichloropalladium(II), complex with dichloromethane
PE: petroleum ether
PPh₃ triphenylphosphine
prep-HPLC: preparative high performance liquid chromatography
prep-TLC: preparative thin layer chromatography
rt: room temperature
RT: retention time
RuCl[(R,R)-TsDPEN](p-cymene) ((R,R)-2-amino-1,2-diphenylethyl)[(4-tolyl)sulfonyl]amido](p-cymene) Ruthenium(II)chloride
SFC: supercritical fluid chromatography
TFA: trifluoroacetic acid
TLC: thin layer chromatography
TMSCHF₂ (difluoromethyl)trimethylsilane
v/v volume ratio

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (MH)⁺.

NMR Spectra were obtained using Bruker Avance 400 MHz or 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Example 1

### 5-(1,3-dimethylpyrazolo[3,4-c]pyridazin-5-yl)-1H-pyrimidine-2,4-dione

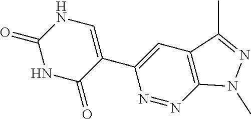 [SMILES: Cc1nn(C)c2nnc(-c3c[nH]c(=O)[nH]c3=O)cc12]

The titled compound was synthesized according to the following scheme:

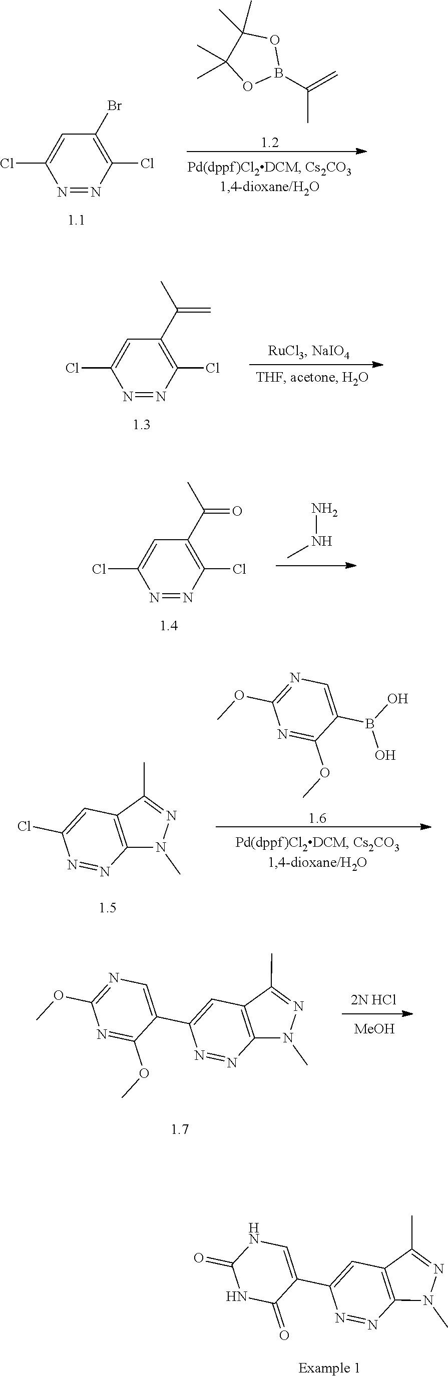

### Step (a): Preparation of 3,6-dichloro-4-isopropenyl-pyridazine (Compound 1.3)

To a solution of 4-bromo-3,6-dichloro-pyridazine (compound 1.1, 0.90 g, 3.95 mmol) in 1,4-dioxane (10 mL) and water (1 mL) was added 2-isopropenyl-4,4,5,5-tetramethyl-1,3,2-dioxaborolane (compound 1.2, 0.73 g, 4.34 mmol), Cs₂CO₃ (2.57 g, 7.9 mmol) and Pd(dppf)Cl₂·DCM (289.0 mg, 0.39 mmol). The resultant mixture was degassed and purged with N₂ for three times, and then the mixture was stirred at 80° C. for 7 h under N₂ atmosphere. After being cooled to room temperature, the reaction mixture was diluted with water (50 mL), extracted with EA (50 mL) twice. The combined organic layer was washed with brine (20 mL), dried over Na₂SO₄, filtered and concentrated in vacuo. The residue was purified by silica gel column chromatography (eluent: PE/EA, from 20/1 to 10/1) to afford compound 1.3 (0.70 g). ¹H NMR (400 MHz, DMSO-d₆) δ=8.01-7.91 (m, 1H), 5.61-5.44 (m, 1H), 5.39-5.21 (m, 1H), 2.13-2.07 (m, 3H).

### Step (b): Preparation of 1-(3,6-dichloropyridazin-4-yl)ethanone (Compound 1.4)

To a 50 mL round-bottom flask equipped with a magnetic stir bar was added 3,6-dichloro-4-isopropenyl-pyridazine (compound 1.3, 0.60 g, 3.17 mmol) followed by the addition of water (10 mL), THF (10 mL) and acetone (10 mL). Then sodium metaperiodate (2.04 g, 9.52 mmol) and Ruthenium(III) chloride hydrate (65.83 mg, 0.32 mmol) were added into the mixture at room temperature. The flask was then evacuated and backfilled with nitrogen for three times. The mixture was stirred at room temperature for another 16 h. The reaction was quenched with saturated Na₂SO₃ aqueous solution (30 mL), extracted with EA (40 mL) for three times. The combined organic layer was washed with brine (30 mL), dried over Na₂SO₄, filtered and concentrated in vacuo. The residue was purified by silica gel column chromatography (eluent: PE/EA, from 100/1 to 5/1) to afford compound 1.4 (0.40 g). ¹H NMR (400 MHz, DMSO-d₆) δ=8.29 (s, 1H), 2.58 (s, 3H).

### Step (c): Preparation of 5-chloro-1,3-dimethyl-pyrazolo[3,4-c]pyridazine (Compound 1.5)

The mixture of 1-(3,6-dichloropyridazin-4-yl)ethanone (compound 1.4, 0.30 g, 1.57 mmol) and methylhydrazine (0.22 g, 4.71 mmol) in 1-butanol (3 mL) was heated at 150° C. in a microwave reactor for 1 h. After being cooled to room temperature, the reaction mixture was diluted with 1 M HCl (20 mL), and extracted with EA (50 mL) twice. The combined organic layer was washed with brine (20 mL), dried over Na₂SO₄, filtered and concentrated in vacuo to afford compound 1.5 (0.40 g) which was used directly for the next step without further purification. ¹H NMR (400 MHz, DMSO-d₆) δ=8.47 (s, 1H), 4.16 (s, 3H), 2.55 (s, 3H).

### Step (d): Preparation of 5-(2,4-dimethoxypyrimidin-5-yl)-1,3-dimethyl-pyrazolo[3,4-c]pyridazine (Compound 1.7)

To a solution of 5-chloro-1,3-dimethyl-pyrazolo[3,4-c]pyridazine (compound 1.5, 0.15 g, 0.82 mmol) in 1,4-dioxane (1 mL) and water (0.1 mL) was added 2,4-dimethoxypyrimidine-5-boronic acid (compound 1.6, 0.15 g, 0.82 mmol), Cs₂CO₃ (0.53 g, 1.64 mmol) and Pd(dppf)Cl₂·DCM (0.06 g, 0.08 mmol). The resultant mixture was degassed and purged with N₂ for three times, and then the mixture was stirred at 100° C. for 1 h under N₂ atmosphere. After being cooled to room temperature, the reaction mixture was diluted with water, extracted EA (20 mL) for three times. The combined organic layer was washed with brine (20 mL), dried over Na₂SO₄, filtered and concentrated in vacuo. The residue was purified by silica gel column chromatography (eluent: PE/EA, from 30/1 to 5/1) to afford compound 1.7 (70.0 mg). ¹H NMR (400 MHz, DMSO-d₆) δ=8.86 (s, 1H), 8.48 (s, 1H), 4.21 (s, 3H), 4.04 (s, 3H), 4.01 (s, 3H), 2.60 (s, 3H).

### Step (e): Preparation of 5-(1,3-dimethylpyrazolo[3,4-c]pyridazin-5-yl)-1H-pyrimidine-2,4-dione (Example 1)

To a solution of 5-(2,4-dimethoxypyrimidin-5-yl)-1,3-dimethyl-pyrazolo[3,4-c]pyridazine (compound 1.7, 30.0 mg, 0.1 mmol) in methanol (0.1 mL) was added 2M HCl (1.0 mL). The reaction mixture was stirred at 60° C. for 1 h, then concentrated to afford a crude product, which was purified by pre-HPLC to afford Example 1 (9.5 mg). MS: calc'd 259.1 [(M+H)⁺], measured 259.2 [(M+H)⁺]. ¹H NMR (400 MHz, DMSO-d₆) δ=11.51 (s, 1H), 11.45 (br d, J=5.6 Hz, 1H), 8.67 (s, 1H), 8.33 (d, J=6.4 Hz, 1H), 4.18 (s, 3H), 2.57 (s, 3H)

### Example 2

### 5-(3-cyclopropyl-1-methyl-pyrazolo[3,4-c]pyridazin-5-yl)-1H-pyrimidine-2,4-dione

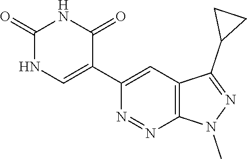 [SMILES: Cn1nc(C2CC2)c2cc(-c3c[nH]c(=O)[nH]c3=O)nnc21]

The titled compound was synthesized according to the following scheme:

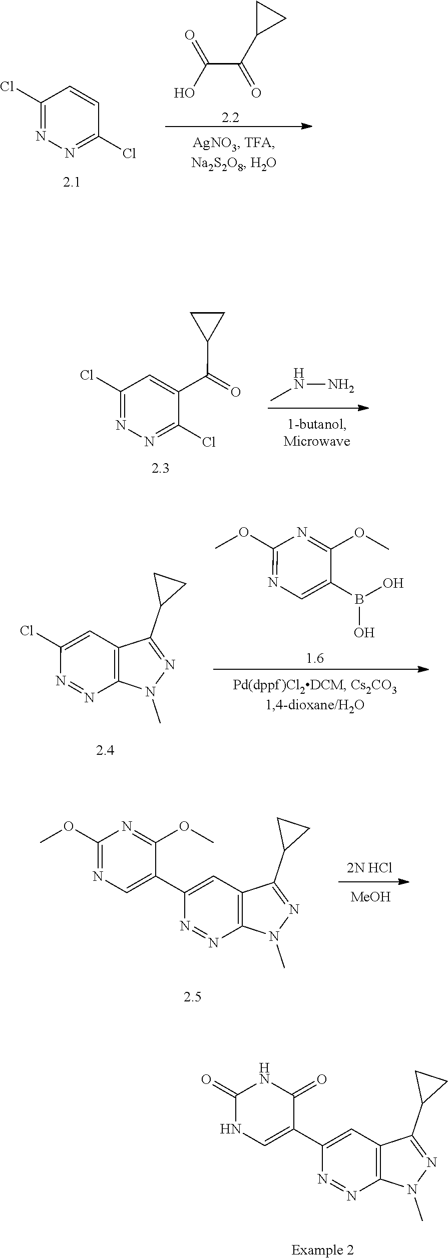

### Step (a): Preparation of cyclopropyl-(3,6-dichloropyridazin-4-yl)methanone (Compound 2.3)

To a mixture of 3,6-dichloropyridazine (compound 2.1, 0.80 g, 5.37 mmol), 2-cyclopropyl-2-oxo-acetic acid (compound 2.2, 0.92 g, 8.05 mmol), silver nitrate (0.18 g, 1.07 mmol) and TFA (0.41 mL, 5.37 mmol) in water (16 mL) was added a solution of Na₂S₂O₈ (1.92 g, 8.05 mmol) in water (8 mL) at 60° C. The resultant mixture was stirred at 60° C. for another 16 h under N₂ atmosphere. After being cooled to room temperature, the reaction mixture was diluted with water (50 mL), extracted with EA (50 mL) for three times. The combined organic layer was washed with brine (20 mL), dried over Na₂SO₄, filtered and concentrated in vacuo. The residue was purified prep-HPLC to afford compound 2.3 (0.20 g). MS: calc'd 217.0 [(M+H)⁺], measured 217.2 [(M+H)⁺].

### Step (b): Preparation of 5-chloro-3-cyclopropyl-1-methyl-pyrazolo[3,4-c]pyridazine (Compound 2.4)

To a solution of cyclopropyl-(3,6-dichloropyridazin-4-yl)methanone (compound 2.3, 0.20 g, 0.92 mmol) in 1-butanol (2 mL) was added methylhydrazine (0.62 mL, 4.69 mmol) to give a yellow solution. The resultant mixture was stirred at 150° C. in a microwave reactor for 1 h. After being cooled to room temperature, the reaction mixture was diluted with 1 M HCl (5 mL), extracted with EA (10 mL) for three times. The combined organic layer was washed with brine (20 mL), dried over Na₂SO₄, filtered and concentrated in vacuo to afford compound 2.4 (0.28 g) which was used directly for the next step without further purification. MS: calc'd 209.0 [(M+H)⁺], measured 209.2 [(M+H)⁺].

### Step (c): Preparation of 5-(3-cyclopropyl-1-methyl-pyrazolo[3,4-c]pyridazin-5-yl)-1H-pyrimidine-2,4-dione (Example 2)

5-(3-cyclopropyl-1-methyl-pyrazolo[3,4-c]pyridazin-5-yl)-1H-pyrimidine-2,4-dione (Example 2) was prepared in analogy to Example 1, by replacing 5-chloro-1,3-dimethyl-pyrazolo[3,4-c]pyridazine (compound 1.5) with 5-chloro-3-cyclopropyl-1-methyl-pyrazolo[3,4-c]pyridazine (compound 2.4) in step (d). 10.8 mg of Example 2 was obtained. MS: calc'd 285.1 [(M+H)⁺], measured 285.3 [(M+H)⁺]. ¹H NMR (400 MHz, DMSO-d₆) δ=8.69 (s, 1H), 8.32 (s, 1H), 4.13 (s, 3H), 2.40-2.32 (m, 1H), 1.11-1.04 (m, 2H), 1.03-0.98 (m, 2H).

### Example 3

### 5-(1-methylpyrazolo[4,3-c]pyridazin-6-yl)-1H-pyrimidine-2,4-dione

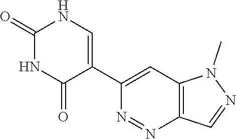 [SMILES: Cn1ncc2nnc(-c3c[nH]c(=O)[nH]c3=O)cc21]

The titled compound was synthesized according to the following scheme:

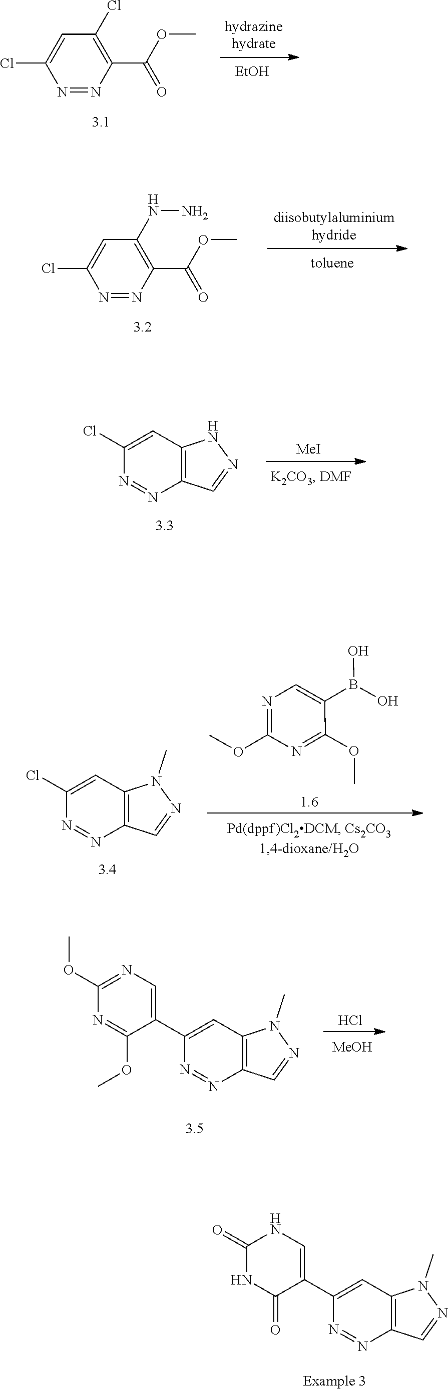

### Step (a): Preparation of methyl 6-chloro-4-hydrazino-pyridazine-3-carboxylate (Compound 3.2)

To a solution of methyl 4,6-dichloropyridazine-3-carboxylate (compound 3.1, 2.0 g, 9.66 mmol) in ethanol (20 mL) was added hydrazine hydrate (1.45 g, 28.97 mmol) at 0° C. The resultant mixture was warmed to 20° C. and stirred at the same temperature for 2 h. The resultant suspension was filtered, and the filter cake was washed with EtOAc (20 mL). The collected solid was concentrated in vacuo to afford compound 3.2 (2.0 g) which was used directly for the next step without further purification. ¹H NMR (400 MHz, Methanol-d₄) δ=7.25 (s, 1H), 3.14 (s, 3H).

### Step (b): Preparation of 6-chloro-1H-pyrazolo[4,3-c]pyridazine (Compound 3.3)

To a solution of methyl 6-chloro-4-hydrazino-pyridazine-3-carboxylate (compound 3.2, 0.15 g, 0.74 mmol) in DCM (3 mL) was added DIBAL-H (1.0 M in THF, 1.11 mL, 1.11 mmol) dropwise at −78° C. The resultant mixture was warmed to room temperature, and stirred at the same temperature for another 1 h. The reaction was quenched by slow addition of H₂O (20 mL), extracted with DCM (30 mL) for three times. The combined organic layer was washed with brine (20 mL), dried over Na₂SO₄, filtered and concentrated in vacuo. The residue was purified by prep-HPLC to afford compound 3.3 (50.0 mg). ¹H NMR (400 MHz, DMSO-d₆) δ=8.73 (d, J=0.8 Hz, 1H), 7.94 (d, J=0.8 Hz, 1H).

### Step (c): Preparation of 6-chloro-1-methyl-pyrazolo[4,3-c]pyridazine (Compound 3.4)

To a solution of 6-chloro-1H-pyrazolo[4,3-c]pyridazine (compound 3.3, 30.0 mg, 0.19 mmol) in DMF (0.5 mL) was added K₂CO₃ (53.65 mg, 0.39 mmol) and iodomethane (68.88 mg, 0.49 mmol). The resultant mixture was stirred at room temperature for 1 hr. The reaction mixture was diluted with water (5 mL), extracted with EA (10 mL) for three times. The combined organic layer was washed with brine (20 mL), dried over Na₂SO₄, filtered and concentrated in vacuo. The residue was purified by silica gel column chromatography (eluent: PE/EA, from 5/1 to 0/1) to afford compound 3.4 (22 mg). MS: calc'd 169.0 [(M+H)⁺], measured 169.0 [(M+H)⁺].

### Step (d): Preparation of 5-(1-methylpyrazolo[4,3-c]pyridazin-6-yl)-1H-pyrimidine-2,4-dione (Example 3)

5-(1-methylpyrazolo[4,3-c]pyridazin-6-yl)-1H-pyrimidine-2,4-dione (Example 3) was prepared in analogy to Example 1, by replacing 5-chloro-1,3-dimethyl-pyrazolo[3,4-c]pyridazine (compound 1.5) with 6-chloro-1-methyl-pyrazolo[4,3-c]pyridazine (compound 3.4) in step (d). 24.9 mg of Example 3 was obtained. MS: calc'd 245.0 [(M+H)⁺], measured 245.0 [(M+H)⁺]. ¹H NMR (400 MHz, DMSO-d₆) δ=11.79-11.47 (m, 2H), 8.81 (s, 1H), 8.63 (s, 1H), 8.59-8.54 (m, 1H), 4.11 (s, 3H).

### Example 4

### 5-(1-methyltriazolo[4,5-c]pyridazin-6-yl)-1H-pyrimidine-2,4-dione

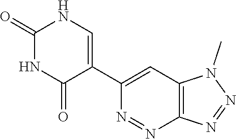 [SMILES: Cn1nnc2nnc(-c3c[nH]c(=O)[nH]c3=O)cc21]

The titled compound was synthesized according to the following scheme:

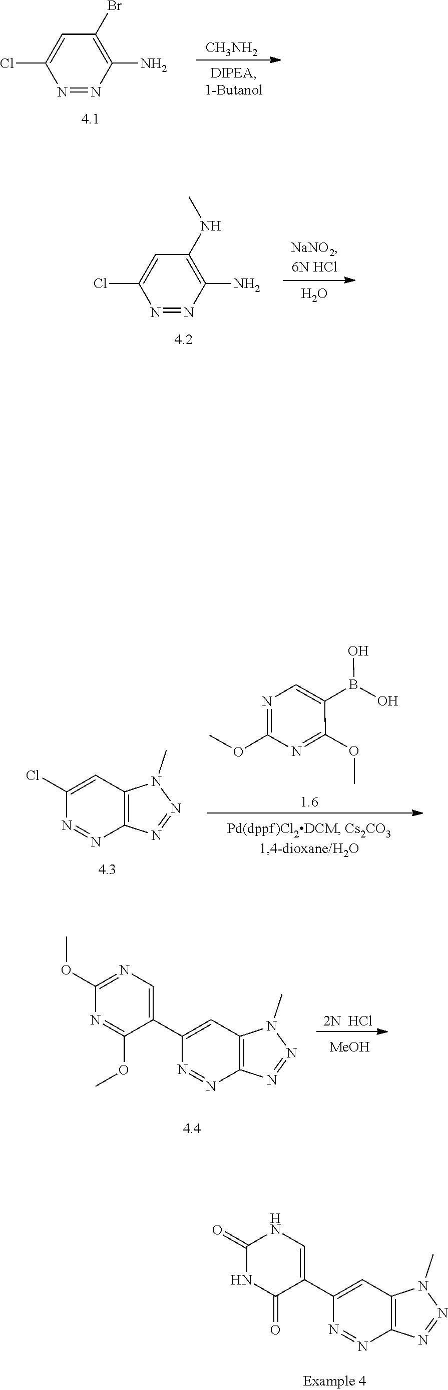

### Step (a): Preparation of 6-chloro-N⁴-methyl-pyridazine-3,4-diamine (Compound 4.2)

To a solution of 4-bromo-6-chloro-pyridazin-3-amine (compound 4.1, 5.0 g, 24.0 mmol) in 1-butanol (20 mL) was added DIEA (8.36 mL, 47.98 mmol) and methylamine (2 M in THF, 24.0 mL, 48.0 mmol) under N₂ atmosphere to give a brown solution. The resultant mixture was stirred at 80° C. for 16 h. The reaction mixture was concentrated and purified by prep-HPLC to afford compound 4.2 (3.5 g). MS: calc'd 159.0 [(M+H)⁺], measured 159.3 [(M+H)⁺].

### Step (b): Preparation of 6-chloro-1-methyl-triazolo[4,5-c]pyridazine (Compound 4.3)

To a solution of 6-chloro-N⁴-methyl-pyridazine-3,4-diamine (compound 4.2, 0.50 g, 3.15 mmol) in 6 M HCl (8 mL) was added a solution of sodium nitrite (0.24 g, 3.47 mmol) in water (2 mL) slowly at 0° C. The resultant mixture was stirred at room temperature for 2 h. After adjusting pH to 8 with sat.NaHCO₃ (aq), the mixture was extracted with EA (20 mL) for three times. The combined organic layer was washed with brine (20 mL), dried over Na₂SO₄, filtered and concentrated in vacuo. The residue was purified by silica gel column chromatography (eluent: PE/EA, from 1/1 to 0/1) to afford compound 4.3 (0.20 g). ¹H NMR (400 MHz, DMSO-d₆) δ=8.69 (s, 1H), 4.36 (s, 3H).

### Step (c): Preparation of 5-(1-methyltriazolo[4,5-c]pyridazin-6-yl)-1H-pyrimidine-2,4-dione (Example 4)

5-(1-methyltriazolo[4,5-c]pyridazin-6-yl)-1H-pyrimidine-2,4-dione (Example 4) was prepared in analogy to Example 1, by replacing 5-chloro-1,3-dimethyl-pyrazolo[3,4-c]pyridazine (compound 1.5) with 6-chloro-1-methyl-triazolo[4,5-c]pyridazine (compound 4.3) in step (d). 70.3 mg of Example 4 was obtained. MS: calc'd 246.0 [(M+H)⁺], measured 246.2 [(M+H)⁺]. ¹H NMR (400 MHz, DMSO-d₆) δ=11.69-11.55 (m, 2H), 8.84 (s, 1H), 8.69-8.58 (m, 1H), 4.38 (s, 3H).

### Example 5

### 5-[1-methyl-3-(1-phenylethyl)pyrazolo[3,4-c]pyridazin-5-yl]-1H-pyrimidine-2,4-dione

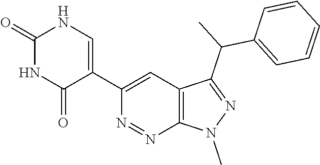 [SMILES: CC(c1ccccc1)c1nn(C)c2nnc(-c3c[nH]c(=O)[nH]c3=O)cc12]

The titled compound was synthesized according to the following scheme:

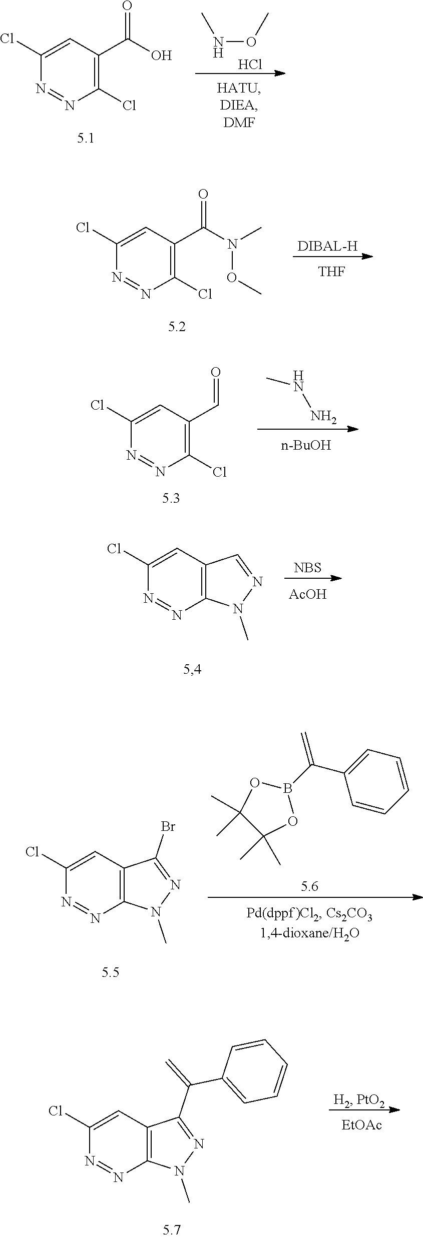

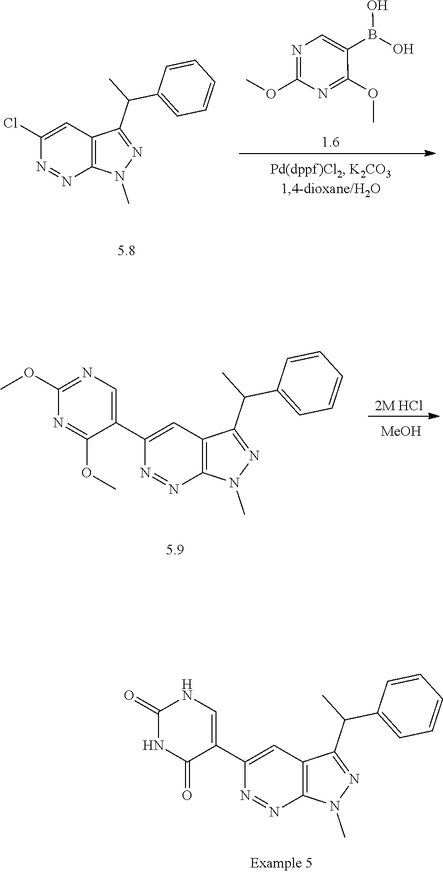

### Step (a): Preparation of 3,6-dichloro-N-methoxy-N-methyl-pyridazine-4-carboxamide (Compound 5.2)

To a solution of 3,6-dichloropyridazine-4-carboxylic acid (compound 5.1, 20.0 g, 103.63 mmol), DIEA (40.73 g, 310.9 mmol) and HATU (59.11 g, 155.45 mmol) in DMF (300 mL) was added N,O-dimethylhydroxylamine hydrochloride (15.16 g, 155.45 mmol). The resultant mixture was stirred at room temperature for 2 h. The reaction was quenched with water (1 L), extracted with EA (300 mL) for three times. The combined organic layer was washed with brine, dried over anhydrous sodium sulfate, filtered and concentrated in vacuo. The residue was purified by silica gel column chromatography (eluent: PE/EA, from 50/1 to 3/1) to afford compound 5.2 (5.0 g). ¹H NMR (400 MHz, Methanol-d₄) δ=8.09 (s, 1H), 3.60 (s, 3H), 3.41 (s, 3H).

### Step (b): Preparation of 3,6-dichloropyridazine-4-carbaldehyde (Compound 5.3)

To a solution of 3,6-dichloro-N-methoxy-N-methyl-pyridazine-4-carboxamide (compound 5.2, 10.0 g, 42.36 mmol) in THF (200 mL) was added drop-wise DIBAL-H (1.0 M in THF, 63.54 mL, 63.54 mmol) at −78° C. The resultant mixture was stirred at the same temperature for 1 h. The reaction was quenched by slow addition of saturated citric acid aqueous solution (200 mL), extracted with EA (100 mL) for three times. The combined organic layer was washed with brine, dried over anhydrous sodium sulfate, filtered and concentrated in vacuo to afford compound 5.3 (10.0 g) which was used directly for the next step without further purification. ¹H NMR (400 MHz, DMSO-d₆) δ=10.12 (s, 1H), 8.26 (s, 1H).

### Step (c): Preparation of 5-chloro-1-methyl-pyrazolo[3,4-c]pyridazine (Compound 5.4)

To a solution of 3,6-dichloropyridazine-4-carbaldehyde (compound 5.3, 10.0 g, 56.5 mmol) in n-butanol (60 mL) was added methylhydrazine (19.52 g, 169.5 mmol), the resultant mixture was stirred at 150° C. for 2 h. After being cooled to room temperature, the mixture was diluted with saturated aqueous ammonium chloride (60 mL), extracted with EA (60 mL) for three times. The combined organic layer was washed with brine, dried over anhydrous sodium sulfate, filtered and concentrated in vacuo. The residue was purified by silica gel column chromatography (eluent: PE/EA, from 100/1 to 2/1) to afford compound 5.4 (2.4 g). ¹H NMR (400 MHz, DMSO-d₆) δ=8.41 (s, 1H), 8.38 (s, 1H), 4.26 (s, 3H).

### Step (d): Preparation of 3-bromo-5-chloro-1-methyl-pyrazolo[3,4-c]pyridazine (Compound 5.5)

To a solution of 5-chloro-1-methyl-pyrazolo[3,4-c]pyridazine (compound 5.5, 1.5 g, 8.9 mmol) in AcOH (25.0 mL) was added NBS (6.33 g, 35.59 mmol), the resultant mixture was stirred at 80° C. for 24 h. The reaction mixture was diluted with water (150 mL), extracted with EA (30 mL) for three times. The combined organic layer was washed with brine, dried over anhydrous sodium sulfate, filtered and concentrated in vacuo. The residue was purified by silica gel column chromatography (eluent: PE/EA, from 50/1 to 10/1) to afford compound 5.5 (1.3 g). ¹H NMR (400 MHz, DMSO-d₆) δ=8.43 (s, 1H), 4.25 (s, 3H).

### Step (e): Preparation of 5-chloro-1-methyl-3-(1-phenylvinyl)pyrazolo[3,4-c]pyridazine (Compound 5.7)

To a solution of 3-bromo-5-chloro-1-methyl-pyrazolo[3,4-c]pyridazine (compound 5.5, 400.0 mg, 1.62 mmol) in 1,4-dioxane (10 mL) and water (1 mL) was added 4,4,5,5-tetramethyl-2-(1-phenylvinyl)-1,3,2-dioxaborolane (compound 5.6, 446.31 mg, 1.94 mmol), Pd(dppf)Cl₂·DCM (118.26 mg, 0.16 mmol) and K₂CO₃ (446.76 mg, 3.23 mmol). The resultant mixture was stirred at 80° C. for 1 h. After being cooled to room temperature, the reaction mixture was diluted with water (50 mL), and extracted with EA (10 mL) for three times. The combined organic layer was washed with brine, dried over anhydrous sodium sulfate, filtered and concentrated in vacuo. The residue was purified by silica gel column chromatography (eluent: PE/EA, from 50/1 to 10/1) to afford compound 5.7 (300.0 mg). ¹H NMR (400 MHz, DMSO-d₆) δ=8.15 (s, 1H), 7.52-7.36 (m, 5H), 6.05 (s, 1H), 5.83 (s, 1H), 4.24 (s, 3H).

### Step (f): Preparation of 5-chloro-1-methyl-3-(1-phenylethyl)pyrazolo[3,4-c]pyridazine (Compound 5.8)

To a 10 mL round-bottom flask equipped with a magnetic stir bar was added 5-chloro-1-methyl-3-(1-phenylvinyl)pyrazolo[3,4-c]pyridazine (compound 5.7, 140.0 mg, 0.52 mmol), followed by the addition of EA (4 mL) and PtO₂ (11.74 mg, 0.05 mmol). The flask was then evacuated and backfilled with hydrogen for three times. The mixture was stirred at 25° C. for 1 h under an atmosphere of hydrogen (15 psi). The reaction mixture was filtered through a pad of celite, and the pad was washed with EtOAc (4 mL). The filtrate was concentrated to afford compound 5.8 (140.0 mg) which was used directly for the next step without further purification. MS: calc'd 273.1, 275.1 [(M+H)⁺], measured 273.3, 275.3 [(M+H)⁺].

### Step (g): Preparation of 5-(2,4-dimethoxypyrimidin-5-yl)-1-methyl-3-(1-phenylethyl)pyrazolo[3,4-c]pyridazine (Compound 5.9)

To a solution of 5-chloro-1-methyl-3-(1-phenylethyl)pyrazolo[3,4-c]pyridazine (compound 5.8, 140.0 mg, 0.51 mmol) in 1,4-dioxane (2 mL) and water (0.2 mL) were added 2,4-dimethoxypyrimidine-5-boronic acid (compound 1.6, 188.86 mg, 1.03 mmol), Pd(dppf)Cl₂·DCM (37.55 mg, 0.05 mmol) and Cs₂CO₃ (334.48 mg, 1.03 mmol). The resultant mixture was stirred at 80° C. for 1 h. After being cooled to room temperature, the reaction mixture was diluted with water (5 mL), and extracted with EA (5 mL) for three times. The combined organic layer was washed with brine, dried over anhydrous sodium sulfate, filtered and concentrated in vacuo. The residue was purified by silica gel column chromatography (eluent: PE/EA, from 50/1 to 10/1) to afford compound 5.9 (160.0 mg). ¹H NMR (400 MHz, DMSO-d₆) δ=8.84 (s, 1H), 7.98 (s, 1H), 7.42-7.24 (m, 5H), 4.66 (d, J=7.2 Hz, 1H), 4.26 (s, 3H), 3.98 (s, 3H), 3.93 (s, 3H), 1.77 (d, J=7.2 Hz, 3H).

### Step (h): Preparation of 5-[1-methyl-3-(1-phenylethyl)pyrazolo[3,4-c]pyridazin-5-yl]-1H-pyrimidine-2,4-dione (Example 5)

The mixture of 5-(2,4-dimethoxypyrimidin-5-yl)-1-methyl-3-(1-phenylethyl)pyrazolo[3,4-c]pyridazine (compound 5.9, 140.0 mg, 0.37 mmol) and 2M HCl (7.0 mL) was stirred at 50° C. for 1 h, then concentrated to afford a crude product, which was purified by pre-HPLC to afford Example 5 (37.4 mg). MS: calc'd 349.1 [(M+H)⁺], measured 349.3 [(M+H)⁺]. ¹H NMR (400 MHz, DMSO-d₆) δ=11.40 (br d, J=2.0 Hz, 2H), 8.38 (s, 1H), 8.29 (s, 1H), 7.41-7.15 (m, 5H), 4.65 (d, J=7.2 Hz, 1H), 4.23 (s, 3H), 1.74 (d, J=7.2 Hz, 3H).

### Example 6

### 5-[1-methyl-3-(1-phenylcyclopropyl)pyrazolo[3,4-c]pyridazin-5-yl]-1H-pyrimidine-2,4-dione

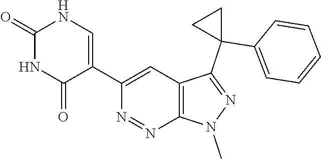 [SMILES: Cn1nc(C2(c3ccccc3)CC2)c2cc(-c3c[nH]c(=O)[nH]c3=O)nnc21]

The titled compound was synthesized according to the following scheme:

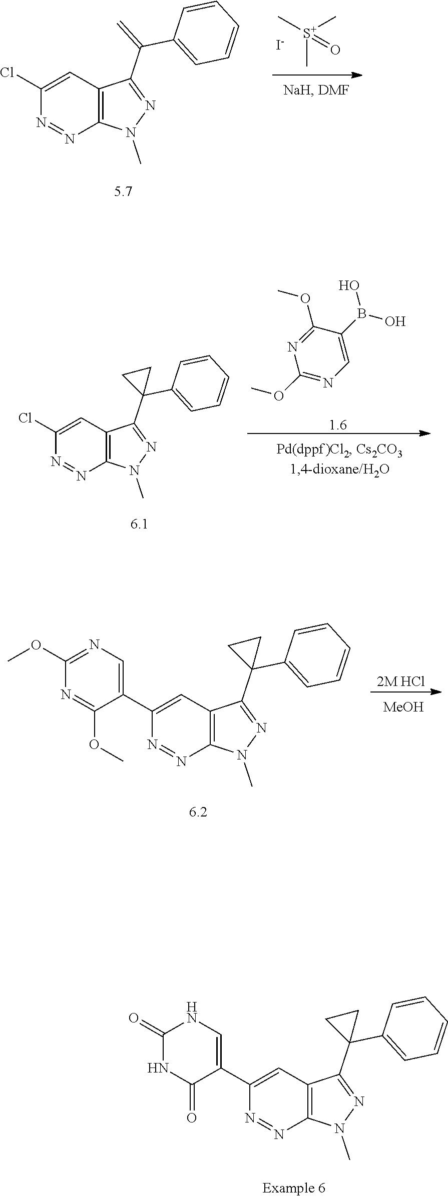

### Step (a): Preparation of 5-chloro-1-methyl-3-(1-phenylcyclopropyl)pyrazolo[3,4-c]pyridazine (Compound 6.1)

To an 8 mL vial equipped with a magnetic stir bar was added NaH (60% dispersion in mineral oil, 59.18 mg, 1.48 mmol) followed by the addition of DMF (3 mL). Then trimethylsulfoxonium iodide (487.74 mg, 2.22 mmol) was added into the mixture. The flask was evacuated and backfilled with nitrogen for three times. The resultant mixture was stirred at 25° C. for 1 h under an atmosphere of nitrogen. Then 5-chloro-1-methyl-3-(1-phenylvinyl)pyrazolo[3,4-c]pyridazine (compound 5.7, 200.0 mg) was added into the mixture at 25° C. The reaction mixture was stirred at 25° C. for another 1 h, quenched with water (20 mL), and extracted with EA (20 mL) for three times. The combined organic layer was washed with brine, dried over anhydrous sodium sulfate, filtered and concentrated in vacuo. The residue was purified by silica gel column chromatography (eluent: PE/EA, from 100/1 to 10/1) to afford compound 6.1 (80.0 mg). MS: calc'd 285.1 [(M+H)⁺], measured 285.3 [(M+H)⁺].

### Step (b): Preparation of 5-[1-methyl-3-(1-phenylcyclopropyl)pyrazolo[3,4-c]pyridazin-5-yl]-1H-pyrimidine-2,4-dione (Example 6)

5-[1-methyl-3-(1-phenylcyclopropyl)pyrazolo[3,4-c]pyridazin-5-yl]-1H-pyrimidine-2,4-dione (Example 6) was prepared in analogy to Example 5, by replacing 5-chloro-1-methyl-3-(1-phenylethyl)pyrazolo[3,4-c]pyridazine (compound 5.8) with 5-chloro-1-methyl-3-(1-phenylcyclopropyl)pyrazolo[3,4-c]pyridazine (compound 6.1) in step (g). 37.4 mg of Example 6 was obtained. MS: calc'd 361.1 [(M+H)⁺], measured 361.3 [(M+H)⁺]. ¹H NMR (400 MHz, DMSO-d₆) δ=11.41 (s, 1H), 11.37 (br d, J=6.0 Hz, 1H), 8.30 (d, J=6.0 Hz, 1H), 8.11 (s, 1H), 7.37-7.22 (m, 5H), 4.20 (s, 3H), 1.60-1.51 (m, 2H), 1.47-1.38 (m, 2H).

### Example 7

### 5-[3-(2-benzylpyrazol-3-yl)-1-methyl-pyrazolo[3,4-c]pyridazin-5-yl]-1H-pyrimidine-2,4-dione

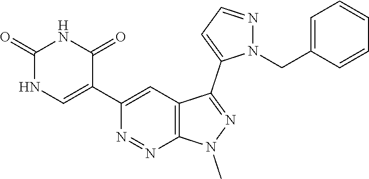 [SMILES: Cn1nc(-c2ccnn2Cc2ccccc2)c2cc(-c3c[nH]c(=O)[nH]c3=O)nnc21]

The titled compound was synthesized according to the following scheme:

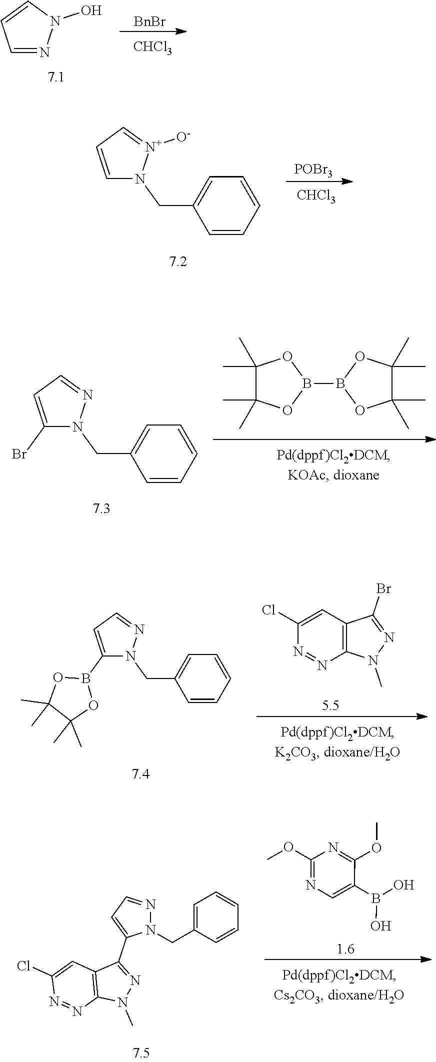

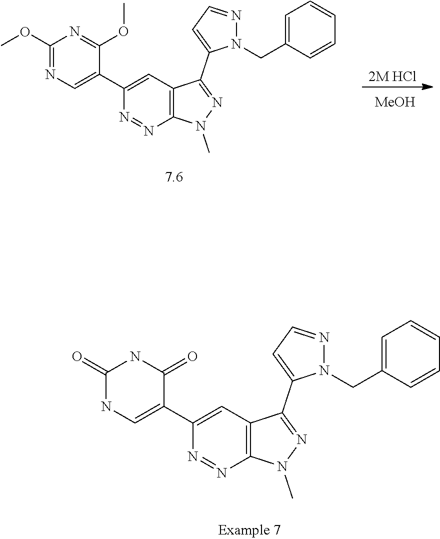 [SMILES: "MeOH" ; COc1ncc(-c2cc3c(-c4ccnn4Cc4ccccc4)nn(C)c3nn2)c(OC)n1 ; "2M HCl" ; Cn1nc(-c2ccnn2Cc2ccccc2)c2cc(-c3c[nH]c(=O)[nH]c3=O)nnc21]

### Example 7

### Step (a): Preparation of 1-benzyl-2-oxido-pyrazol-2-ium (Compound 7.2)

The mixture of 1-hydroxypyrazole (4.0 g, 47.57 mmol) and benzyl bromide (7.36 mL, 61.85 mmol) in chloroform (30 mL) was heated at reflux overnight. After being cooled to room temperature, the mixture was poured into toluene (100 ml) and extracted with 12 M HCl (5 mL) for three times. The combined aqueous layer was washed with toluene (20 mL) and cautiously basified with 33% NaOH aqueous solution to pH>10 under an ice bath. The aqueous layer was extracted with CHCl₃ (20 mL) for three times. After concentration, the crude product compound 7.2 (5.7 g) was obtained and was used into the next step without further purification. MS: calc'd 175.1 [(M+H)⁺], measured 175.2 [(M+H)⁺].

### Step (b): Preparation of 1-benzyl-5-bromo-pyrazole (Compound 7.3)

To a solution of 1-benzyl-2-oxido-pyrazol-2-ium (compound 7.2, 2.5 g, 14.35 mmol) in chloroform (10 mL) was added dropwise a solution of POBr₃ (8229.05 mg, 28.7 mmol, 2.0 eq) in chloroform (10 mL) at 0° C. The resultant mixture was heated to 50° C. and stirred for 2.5 h under an atmosphere of nitrogen. The mixture was evaporated to remove chloroform and the pH was adjusted to 7-8 by addition of sat. aq. NaHCO₃ solution. The aqueous solution was extracted with DCM (30 mL) for three times. The combined organic layer was washed with brine, dried over anhydrous sodium sulfate, filtered, and concentrated. The residue was purified by flash chromatography (silica gel, 80 g, 0% to 25% EA in PE) to afford compound 7.3 (5.2 g). MS: calc'd 237.0, 239.0 [(M+H)⁺], measured 237.0, 239.0 [(M+H)⁺]. ¹H NMR (400 MHz, CDCl₃) δ=7.95 (d, J=2.4 Hz, 1H), 7.69-7.58 (m, 5H), 6.72 (d, J=2.0 Hz, 1H), 5.8 (s, 2H).

### Step (c): Preparation of 1-benzyl-5-(4,4,5,5-tetramethyl-1,3,2-dioxaborolan-2-yl)pyrazole (Compound 7.4)

To a solution of 1-benzyl-5-bromo-pyrazole (compound 7.3, 150.0 mg, 0.63 mmol) in 1,4-dioxane (2 mL) were added bis(pinacolato)diboron (160.65 mg, 0.63 mmol), KOAc (124.18 mg, 1.27 mmol) and Pd(dppf)Cl₂·DCM (46.29 mg, 0.06 mmol). The resultant mixture was stirred at 80° C. for 1 h under an atmosphere of nitrogen. The reaction mixture was concentrated to afford a crude product, which was purified by flash chromatography (silica gel, 12 g, 0% to 50% EA in PE) to afford compound 7.4 (52.0 mg). MS: calc'd 285.1 [(M+H)⁺], measured 203.4, [(M+H—C₆H₁₀)⁺].

### Step (d): Preparation of 3-(2-benzylpyrazol-3-yl)-5-chloro-1-methyl-pyrazolo[3,4-c]pyridazine (Compound 7.5)

To a solution of 1-benzyl-5-(4,4,5,5-tetramethyl-1,3,2-dioxaborolan-2-yl)pyrazole (compound 7.4, 137.79 mg, 0.48 mmol)) in 1,4-dioxane (2 mL) and water (0.2 mL) were added 3-bromo-5-chloro-1-methyl-pyrazolo[3,4-c]pyridazine (compound 5.5, 120.0 mg, 0.48 mmol), K₂CO₃ (134.03 mg, 0.97 mmol) and Pd(dppf)Cl₂·DCM (35.47 mg, 0.05 mmol). The resultant mixture was stirred at 80° C. for 4 h under an atmosphere of nitrogen. After being cooled to room temperature, the reaction mixture was diluted with water (20 mL), and extracted with EA (20 mL) for three times. The combined organic layer was washed with brine, dried over anhydrous sodium sulfate, filtered and concentrated in vacuo. The residue was purified by silica gel column chromatography (eluent: PE/EA, from 100/1 to 10/1) to afford compound 7.5 (130.0 mg). MS: calc'd 325.1 [(M+H)⁺], measured 325.3 [(M+H)⁺].

### Step (e): Preparation of 5-[3-(2-benzylpyrazol-3-yl)-1-methyl-pyrazolo[3,4-c]pyridazin-5-yl]-1H-pyrimidine-2,4-dione (Example 7)

5-[3-(2-benzylpyrazol-3-yl)-1-methyl-pyrazolo[3,4-c]pyridazin-5-yl]-1H-pyrimidine-2,4-dione (Example 7) was prepared in analogy to Example 5, by replacing 5-chloro-1-methyl-3-(1-phenylethyl)pyrazolo[3,4-c]pyridazine (compound 5.8) with 3-(2-benzylpyrazol-3-yl)-5-chloro-1-methyl-pyrazolo[3,4-c]pyridazine (compound 7.5) in step (g). 16.0 mg of Example 7 was obtained. MS: calc'd 401.1 [(M+H)⁺], measured 401.4 [(M+H)⁺]. ¹H NMR (400 MHz, DMSO-d₆) δ=11.56 (s, 1H), 11.51 (br d, J=6.0 Hz, 1H), 8.82 (s, 1H), 8.38 (d, J=6.0 Hz, 1H), 7.75 (d, J=2.0 Hz, 1H), 7.35-7.10 (m, 5H), 6.98 (d, J=2.0 Hz, 1H), 5.83 (s, 2H), 4.33 (s, 3H).

### Example 8

### 5-(3-isopropoxy-1-methyl-pyrazolo[3,4-c]pyridazin-5-yl)-1H-pyrimidine-2,4-dione

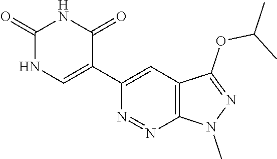 [SMILES: CC(C)Oc1nn(C)c2nnc(-c3c[nH]c(=O)[nH]c3=O)cc12]

The titled compound was synthesized according to the following scheme:

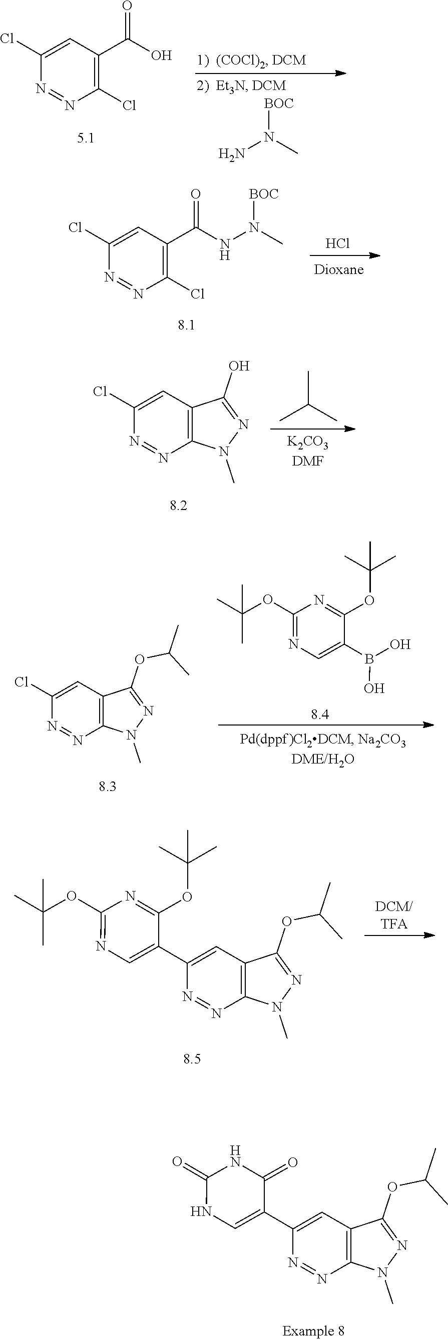

### Step (a): Preparation of tert-butyl N-[(3,6-dichloropyridazine-4-carbonyl)amino]-N-methyl-carbamate (Compound 8.1)

3,6-dichloropyridazine-4-carboxylic acid (compound 5.1, 1.0 g, 5.18 mmol) was suspended in dichloromethane (12.5 mL) and catalytic amount of DMF (5 drops). The mixture was cooled to 0° C. Oxalyl chloride (613.86 μL, 7.25 mmol) was slowly added and continued to stir at 0° C. for 20 min. The resultant mixture was warmed to 25° C. and stirred for 20 min until a clear solution was obtained. The solvent and residual oxalyl chloride were removed entirely under vacuum and the residue was re-dissolved in dichloromethane (12.5 mL). tert-butyl N-amino-N-methyl-carbamate (833.25 mg, 5.7 mmol) and triethylamine (2.22 mL, 15.54 mmol) was dissolved in dichloromethane (12.5 mL) and was cooled to 0° C. The above solution of acyl chloride was added dropwise under 0° C., then the solution was slowly warmed to room temperature and stirred for 4 hrs until the completion of reaction. The mixture was diluted with dichloromethane (30 mL) and was washed with water (20 mL×2), brine (20 mL×2), and dried over Na₂SO₄. The organic phase was concentrated to get a crude compound 8.1, which was used directly for the next step without further purification. MS: calc'd 321.1 [(M+H)⁺], measured 321.1 [(M+H)⁺].

### Step (b) Preparation of 5-chloro-1-methyl-pyrazolo[3,4-c]pyridazin-3-ol (Compound 8.2)

The mixture of above crude compound 8.1 and HCl/dioxane (4 M, 25 mL) was stirred at 55° C. for another 10 h. The desired product compound 8.2 was filtered out from the solution and dried to obtain a red solid (762.0 mg). MS: calc'd 185.0 [(M+H)⁺], measured 185.1 [(M+H)⁺]

### Step (c): Preparation of 5-chloro-3-isopropoxy-1-methyl-pyrazolo[3,4-c]pyridazine (Compound 8.3)

To a solution of 5-chloro-1-methyl-pyrazolo[3,4-c]pyridazin-3-ol (compound 8.2, 100.0 mg, 0.54 mmol) in DMF (2.0 mL) was added K₂CO₃ (225.4 mg, 1.63 mmol) and 2-iodopropane (275.4 mg, 1.63 mmol). The resultant mixture was heated at 70° C. for 16 h. After being cooled to room temperature, the mixture was diluted with EA (50 mL), washed with water (20 mL×2), brine (20 mL×2), dried over anhydrous sodium sulfate, filtrated and concentrated in vacuo. The residue was purified by silica column chromatography (eluent: PE/EA, from 10/1 to 3/1) to afford compound 8.3 (40.0 mg). MS: calc'd 227.1 [(M+H)⁺], measured 227.1 [(M+H)⁺].

### Step (d): Preparation of 5-(2,4-ditert-butoxypyrimidin-5-yl)-3-isopropoxy-1-methyl-pyrazolo[3,4-c]pyridazine (Compound 8.5)

To a Schlenk flask was added 5-chloro-3-isopropoxy-1-methyl-pyrazolo[3,4-c]pyridazine (compound 8.3, 40 mg, 0.176 mmol), (2,4-ditert-butoxypyrimidin-5-yl)boronic acid (compound 8.4, 70.9 mg, 0.265 mmol), Na₂CO₃ (74.8 mg, 4.0 eq), Pd(dppf)Cl₂·DCM (12.9 mg, 0.017 mmol), DME (4.0 mL) and water (1.0 mL). The flask was evacuated and backfilled with N₂ for three times, after which the resultant mixture was stirred at 80° C. for 5 hrs.

After being cooled to room temperature, the mixture was diluted with EA (50 mL), washed with water (20 mL×2), brine (20 mL×2), dried over anhydrous sodium sulfate, filtrated and concentrated in vacuo. The residue was purified by silica column chromatography (eluent: PE/EA, from 10/1 to 3/1) to afford compound 8.5 (45.0 mg).

### Step (e) Preparation of 5-(3-isopropoxy-1-methyl-pyrazolo[3,4-c]pyridazin-5-yl)-1H-pyrimidine-2,4-dione (Example 8)

To a solution of 5-(2,4-ditert-butoxypyrimidin-5-yl)-3-isopropoxy-1-methyl-pyrazolo[3,4-c]pyridazine (compound 8.5, 45.0 mg, 0.11 mmol) in dichloromethane (2.0 mL) was added TFA (0.1 mL). After stirred at room temperature for 20 min, the reaction mixture was concentrated to afford a crude product, which was purified by pre-HPLC to afford Example 8 (32.0 mg). MS: calc'd 303.1 [(M+H)⁺], measured 303.1 [(M+H)⁺]. ¹H NMR (400 MHz, DMSO-d₆) δ=11.54-11.50 (m, 1H), 11.48-11.38 (m, 1H), 8.54 (s, 1H), 8.34 (d, J=6.3 Hz, 1H), 5.11 (multiple, J=6.1 Hz, 1H), 4.06 (s, 3H), 1.42 (d, J=6.1 Hz, 6H).

### Example 9

### 5-(3-chloro-1-methyl-pyrazolo[4,3-c]pyridazin-6-yl)-1H-pyrimidine-2,4-dione

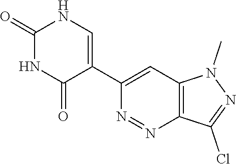 [SMILES: Cn1nc(Cl)c2nnc(-c3c[nH]c(=O)[nH]c3=O)cc21]

The titled compound was synthesized according to the following scheme:

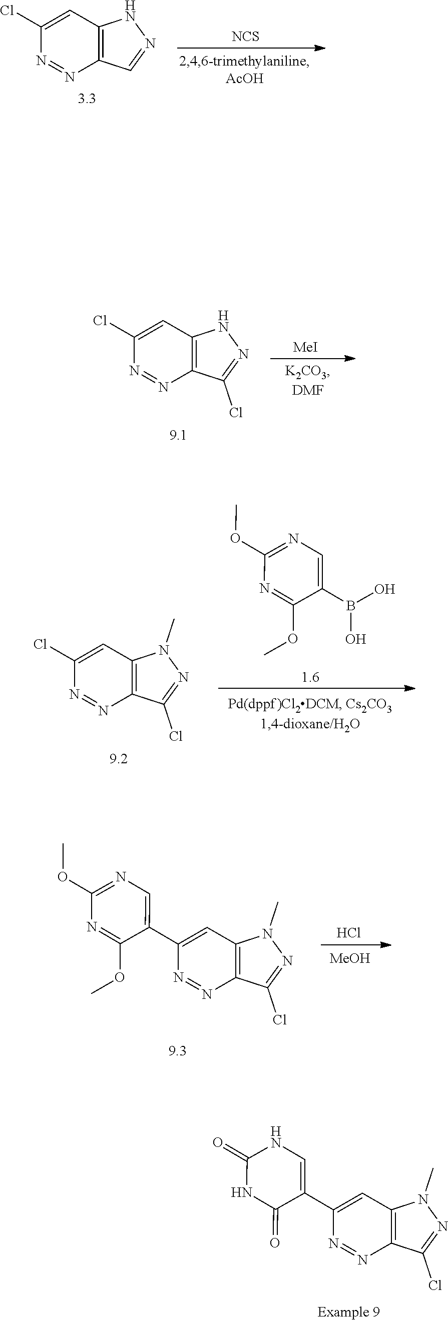

### Step (a) Preparation of 3,6-dichloro-1H-pyrazolo[4,3-c]pyridazine (Compound 9.1)

To an 8 mL round-bottom flask equipped with a magnetic stir bar was added 6-chloro-1H-pyrazolo[4,3-c]pyridazine (compound 3.3, 400.0 mg, 2.59 mmol), followed by the addition of AcOH (10.0 mL). Then NCS (1.73 g, 12.94 mmol) and 2,4,6-trimethylaniline (34.99 mg, 0.26 mmol) were added into the mixture at 25° C. The mixture was stirred at 70° C. for 1 h. The reaction was quenched by slow addition of H₂O (20 mL), extracted with ethyl acetate (20 mL) for three times. The combined organic layer was washed with brine (20 mL), dried over anhydrous sodium sulfate, filtered and concentrated in vacuo. The residue was purified by silica gel column chromatography (eluent: PE/EA, from 1/100 to 1/2) to afford compound 9.1 (700.0 mg). MS: calc'd 189.1 [(M+H)⁺], measured 189.2 [(M+H)⁺].

### Step (b) Preparation of 3,6-dichloro-1-methyl-pyrazolo[4,3-c]pyridazine (Compound 9.2)

To a solution of 3,6-dichloro-1H-pyrazolo[4,3-c]pyridazine (compound 9.1, 500.0 mg, 2.65 mmol) in DMF (15 mL) was added iodomethane (563.25 mg, 3.97 mmol) and K₂CO₃ (731.27 mg, 5.29 mmol). The resultant mixture was stirred at 25° C. for 1 h. The reaction mixture was quenched by slow addition of H₂O (20 mL), extracted with EA (15 mL) for three times. The combined organic layer was washed with brine (15 mL), dried over anhydrous sodium sulfate, filtered and concentrated in vacuo. The residue was purified by silica gel column chromatography (eluent: PE/EA, from 100/1 to 1/1) to afford compound 9.2 (150.0 mg). MS: calc'd 203.0 [(M+H)⁺], measured 203.0 [(M+H)⁺]. ¹H NMR of (400 MHz, DMSO-d₆) δ=8.47 (s, 1H), 4.06 (s, 3H).

### Step (c) Preparation of 5-(3-chloro-1-methyl-pyrazolo[4,3-c]pyridazin-6-yl)-1H-pyrimidine-2,4-dione (Example 9)

5-(3-chloro-1-methyl-pyrazolo[4,3-c]pyridazin-6-yl)-1H-pyrimidine-2,4-dione (Example 9) was prepared in analogy to Example 1, by replacing 5-chloro-1,3-dimethyl-pyrazolo[3,4-c]pyridazine (compound 1.5) with 3,6-dichloro-1-methyl-pyrazolo[4,3-c]pyridazine (compound 9.2) in step (d). 20.0 mg of Example 9 was obtained. MS: calc'd 279.0 [(M+H)⁺], measured 279.2 [(M+H)⁺]. ¹H NMR (400 MHz, DMSO-d₆) δ=11.59 (br s, 2H), 8.64 (s, 1H), 8.60 (s, 1H), 4.07 (s, 3H).

### Example 10

### 5-[1-methyl-3-(1-phenylethoxy)pyrazolo[3,4-c]pyridazin-5-yl]-1H-pyrimidine-2,4-dione

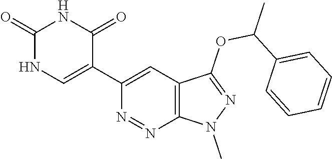 [SMILES: CC(Oc1nn(C)c2nnc(-c3c[nH]c(=O)[nH]c3=O)cc12)c1ccccc1]

5-[1-methyl-3-(1-phenylethoxy)pyrazolo[3,4-c]pyridazin-5-yl]-1H-pyrimidine-2,4-dione (Example 10) was prepared in analogy to Example 8, by replacing 2-iodopropane with 1-bromoethylbenzene in step (c). 34.9 mg of Example 10 was obtained. MS: calc'd 365.1 [(M+H)⁺], measured 365.2 [(M+H)⁺]. ¹H NMR (400 MHz, DMSO-d₆) δ=11.51 (d, J=1.4 Hz, 1H), 11.44-11.39 (m, 1H), 8.61 (s, 1H), 8.33 (d, J=6.3 Hz, 1H), 7.51 (d, J=7.1 Hz, 2H), 7.40-7.34 (m, 2H), 7.30 (d, J=7.3 Hz, 1H), 6.02 (d, J=6.4 Hz, 1H), 4.02 (s, 3H), 1.70 (d, J=6.5 Hz, 3H).

### Example 11

### 3-[1-[5-(2,4-dioxo-1H-pyrimidin-5-yl)-1-methyl-pyrazolo[3,4-c]pyridazin-3-yl]oxyethyl]benzonitrile

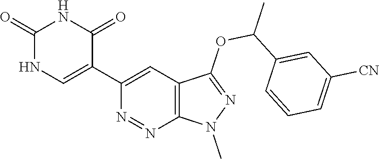 [SMILES: CC(Oc1nn(C)c2nnc(-c3c[nH]c(=O)[nH]c3=O)cc12)c1cccc(C#N)c1]

The titled compound was synthesized according to the following scheme

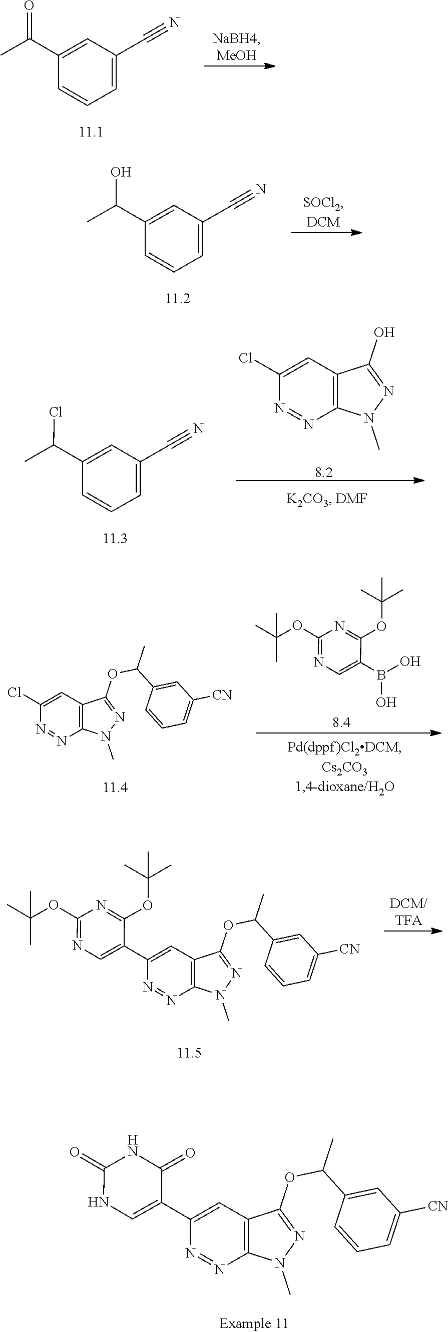

### Step (a): Preparation of 3-(1-hydroxyethyl)benzonitrile (Compound 11.2)

To a solution of 3-cyanoacetophenone (1 g, 6.9 mmol) in methanol (10 mL) was added sodium borohydride (312.7 mg, 8.3 mmol) at 0° C. The resultant mixture was stirred at 0° C. for 30 min. Then the reaction was quenched with 1M HCl (3 mL), diluted with water (100 mL), and extracted with DCM (20 mL) for three times. The combined organic layer was dried over Na₂SO₄ and concentrated in vacuo to afford compound 11.2 (1.09 g) which was used for next step directly.

### Step (b): Preparation of 3-(1-chloroethyl)benzonitrile (Compound 11.3)

To a solution of 3-(1-hydroxyethyl)benzonitrile (compound 11.2, 1.09 g, 7.4 mmol) in DCM (10 mL) was added drop-wise SOCl₂ (1.32 g, 810.9 μL, 11.1 mmol) at 0° C. The resultant mixture was stirred at room temperature for 2 hr. The reaction mixture was concentrated in vacuo to afford compound 11.3 (1.2 g) which was used for next step directly.

### Step (c): Preparation of 3-[1-(5-chloro-1-methyl-pyrazolo[3,4-c]pyridazin-3-yl)oxyethyl]benzonitrile (Compound 11.4)

To a solution of 5-chloro-1-methyl-pyrazolo[3,4-c]pyridazin-3-ol (compound 8.2, 750 mg, 4.1 mmol) in DMF (15 mL) was added 3-(1-chloroethyl)benzonitrile (compound 11.3, 1.01 g, 6.1 mmol) and K₂CO₃ (3.37 g, 24.4 mmol). The resultant mixture was stirred at 70° C. for 2 hr. After being cooled to room temperature, the reaction mixture was diluted with H₂O (100 mL), and extracted with EA (20 mL) for three times. The combined organic layer was washed with brine, dried over Na₂SO₄, filtered and concentrated in vacuo. The residue was purified by flash chromatography (silica gel, 40 g, 0% to 30% EA in PE) to afford compound 11.4 (250 mg). MS: calc'd 314.1 [(M+H)⁺]; measured 314.1 [(M+H)⁺].

### Step (d): Preparation of 3-[1-[5-(2,4-ditert-butoxypyrimidin-5-yl)-1-methyl-pyrazolo[3,4-c]pyridazin-3-yl]oxyethyl]benzonitrile (Compound 11.5)

To a solution of 3-[1-(5-chloro-1-methyl-pyrazolo[3,4-c]pyridazin-3-yl)oxyethyl]benzonitrile (compound 11.4, 250 mg, 796.8 μmol) in 1,4-dioxane (6 mL) and water (1.5 mL) was added (2,4-ditert-butoxypyrimidin-5-yl)boronic acid (compound 8.4, 256.4 mg, 956.2 μmol), Pd(dppf)Cl₂·DCM (65.1 mg, 79.7 μmol) and Na₂CO₃ (337.8 mg, 3.2 mmol). The resultant mixture was stirred at 90° C. under nitrogen for 2 hr. After being cooled to room temperature, the reaction mixture was diluted with H₂O (50 mL), and extracted with EA (10 mL) for three times. The combined organic layer was washed with brine, dried over Na₂SO₄, filtered and concentrated in vacuo. The residue was purified by flash chromatography (silica gel, 12 g, 0% to 30% EA in PE) to afford compound 11.5 (310 mg). MS: calc'd 502.3 [(M+H)⁺]; measured 502.4 [(M+H)⁺].

### Step (e): Preparation of 3-[1-[5-(2,4-dioxo-1H-pyrimidin-5-yl)-1-methyl-pyrazolo[3,4-c]pyridazin-3-yl]oxyethyl]benzonitrile (Example 11)

To a solution of 3-[1-[5-(2,4-ditert-butoxypyrimidin-5-yl)-1-methyl-pyrazolo[3,4-c]pyridazin-3-yl]oxyethyl]benzonitrile (compound 11.5, 310 mg, 618.1 μmol) in dichloromethane (3 mL) was added TFA (500 μL). After being stirred at room temperature for 20 min, the reaction mixture was concentrated to afford a crude product, which was purified by pre-HPLC to afford Example 11 (151.9 mg). MS: calc'd 390.1 [(M+H)⁺]; measured 390.2 [(M+H)⁺]. ¹H NMR (400 MHz, DMSO-d₆) δ=11.54-11.50 (m, 1H), 11.45-11.38 (m, 1H), 8.64 (s, 1H), 8.33 (d, J=6.2 Hz, 1H), 8.01 (s, 1H), 7.88 (d, J=7.9 Hz, 1H), 7.78 (dt, J=7.8, 1.4 Hz, 1H), 7.59 (t, J=7.8 Hz, 1H), 6.07 (q, J=6.3 Hz, 1H), 4.01 (s, 3H), 1.71 (d, J=6.5 Hz, 3H).

### Example 12

### 4-[1-[5-(2,4-dioxo-1H-pyrimidin-5-yl)-1-methyl-pyrazolo[3,4-c]pyridazin-3-yl]oxyethyl]benzonitrile

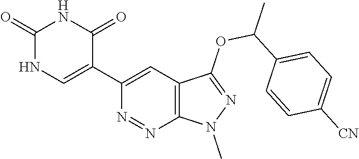 [SMILES: CC(Oc1nn(C)c2nnc(-c3c[nH]c(=O)[nH]c3=O)cc12)c1ccc(C#N)cc1]

4-[1-[5-(2,4-dioxo-1H-pyrimidin-5-yl)-1-methyl-pyrazolo[3,4-c]pyridazin-3-yl]oxyethyl]benzonitrile (Example 12) was prepared in analogy to Example 11, by replacing 3-acetylbenzonitrile (compound 11.1) with 4-acetylbenzonitrile in step (a). MS: calc'd 390.1 [(M+H)⁺], measured 390.2 [(M+H)⁺]. ¹H NMR (400 MHz, DMSO-d₆) δ=11.52 (s, 1H), 11.42 (br s, 1H), 8.64 (s, 1H), 8.34 (s, 1H), 7.87-7.81 (m, 2H), 7.72 (d, J=8.2 Hz, 2H), 6.09 (q, J=6.5 Hz, 1H), 4.00 (s, 3H), 1.70 (d, J=6.5 Hz, 3H).

### Example 13

### 5-[3-[1-(2-chlorophenyl)ethoxy]-1-methyl-pyrazolo[3,4-c]pyridazin-5-yl]-1H-pyrimidine-2,4-dione

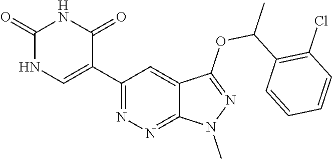 [SMILES: CC(Oc1nn(C)c2nnc(-c3c[nH]c(=O)[nH]c3=O)cc12)c1ccccc1Cl]

5-[3-[1-(2-chlorophenyl)ethoxy]-1-methyl-pyrazolo[3,4-c]pyridazin-5-yl]-1H-pyrimidine-2,4-dione (Example 13) was prepared in analogy to Example 11, by replacing 3-(1-hydroxyethyl)benzonitrile (compound 11.2) with 1-(2-chlorophenyl)ethanol in step (b). MS: calc'd 399.1 [(M+H)⁺]; measured 399.2 [(M+H)⁺]. ¹H NMR (500 MHz, DMSO-d₆) δ=11.54 (s, 1H), 11.44 (br s, 1H), 8.65 (s, 1H), 8.35 (s, 1H), 7.65 (dd, J=7.3, 2.1 Hz, 1H), 7.49 (dd, J=7.6, 1.8 Hz, 1H), 7.40-7.30 (m, 2H), 6.29 (q, J=6.4, 1H), 4.00 (s, 3H), 1.70 (d, J=6.4, 3H).

### Example 14

### 5-[3-[1-(3-chlorophenyl)ethoxy]-1-methyl-pyrazolo[3,4-c]pyridazin-5-yl]-1H-pyrimidine-2,4-dione

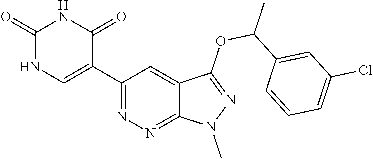 [SMILES: CC(Oc1nn(C)c2nnc(-c3c[nH]c(=O)[nH]c3=O)cc12)c1cccc(Cl)c1]

5-[3-[1-(3-chlorophenyl)ethoxy]-1-methyl-pyrazolo[3,4-c]pyridazin-5-yl]-1H-pyrimidine-2,4-dione (Example 14) was prepared in analogy to Example 11, by replacing replacing 3-(1-hydroxyethyl)benzonitrile (compound 11.2) with 1-(3-chlorophenyl)ethanol in step (b). MS: calc'd 399.1 [(M+H)⁺]; measured 399.2 [(M+H)⁺]. ¹H NMR (500 MHz, DMSO-d₆) δ=11.54 (s, 1H), 11.44 (br s, 1H), 8.63 (s, 1H), 8.34 (s, 1H), 7.58 (t, J=1.7 Hz, 1H), 7.49 (d, J=7.6 Hz, 1H), 7.41 (t, J=7.7 Hz, 1H), 7.37 (d, J=7.8 Hz, 1H), 6.03 (q, J=6.4, 1H), 4.02 (s, 3H), 1.69 (d, J=6.4, 3H).

### Example 15

### 5-[3-[1-(4-chlorophenyl)ethoxy]-1-methyl-pyrazolo[3,4-c]pyridazin-5-yl]-1H-pyrimidine-2,4-dione

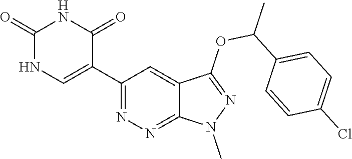 [SMILES: CC(Oc1nn(C)c2nnc(-c3c[nH]c(=O)[nH]c3=O)cc12)c1ccc(Cl)cc1]

5-[3-[1-(4-chlorophenyl)ethoxy]-1-methyl-pyrazolo[3,4-c]pyridazin-5-yl]-1H-pyrimidine-2,4-dione (Example 15) was prepared in analogy to Example 11, by replacing 3-(1-hydroxyethyl)benzonitrile (compound 11.2) with 1-(4-chlorophenyl)ethanol in step (b). MS: calc'd 399.1 [(M+H)⁺]; measured 399.2 [(M+H)⁺]. ¹H NMR (500 MHz, DMSO-d₆) δ=11.54 (d, J=1.7 Hz, 1H), 11.44 (dd, J=6.0, 1.4 Hz, 1H), 8.61 (s, 1H), 8.34 (d, J=6.1 Hz, 1H), 7.60-7.52 (m, 2H), 7.47-7.37 (m, 2H), 6.02 (q, J=6.5 Hz, 1H), 4.01 (s, 3H), 1.68 (d, J=6.6, 3H).

### Example 16

### 5-[3-[1-(4-fluorophenyl)ethoxy]-1-methyl-pyrazolo[3,4-c]pyridazin-5-yl]-1H-pyrimidine-2,4-dione

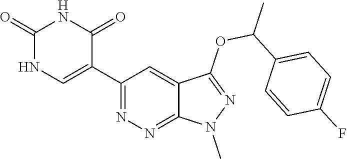 [SMILES: CC(Oc1nn(C)c2nnc(-c3c[nH]c(=O)[nH]c3=O)cc12)c1ccc(F)cc1]

5-[3-[1-(4-fluorophenyl)ethoxy]-1-methyl-pyrazolo[3,4-c]pyridazin-5-yl]-1H-pyrimidine-2,4-dione (Example 16) was prepared in analogy to Example 11, by replacing 3-(1-hydroxyethyl)benzonitrile (compound 11.2) with 1-(4-fluorophenyl)ethanol in step (b). MS: calc'd 383.1 [(M+H)⁺]; measured 383.2 [(M+H)⁺]. ¹H NMR (500 MHz, DMSO-d₆) δ=11.53 (br s, 1H), 11.43 (br s, 1H), 8.60 (s, 1H), 8.34 (s, 1H), 7.62-7.54 (m, 2H), 7.24-7.16 (m, 2H), 6.03 (q, J=6.4 Hz, 1H), 4.02 (s, 3H), 1.69 (d, J=6.6 Hz, 3H).

### Example 17

### 5-[1-methyl-3-[1-(2-pyridyl)ethoxy]pyrazolo[3,4-c]pyridazin-5-yl]-1H-pyrimidine-2,4-dione

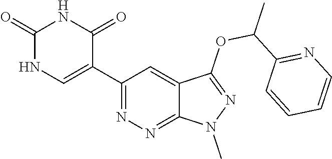 [SMILES: CC(Oc1nn(C)c2nnc(-c3c[nH]c(=O)[nH]c3=O)cc12)c1ccccn1]

5-[1-methyl-3-[1-(2-pyridyl)ethoxy]pyrazolo[3,4-c]pyridazin-5-yl]-1H-pyrimidine-2,4-dione (Example 17) was prepared in analogy to Example 11, by replacing 3-(1-hydroxyethyl)benzonitrile (compound 11.2) with 1-(2-pyridyl)ethanol in step (b). MS: calc'd 366.1 [(M+H)⁺]; measured 366.2 [(M+H)⁺]. ¹H NMR (500 MHz, DMSO-d₆) δ=11.56-11.52 (m, 1H), 11.50-11.39 (m, 1H), 8.64 (s, 1H), 8.57 (d, J=5.1 Hz, 1H), 8.35 (d, J=6.3 Hz, 1H), 7.80 (td, J=7.7, 1.7 Hz, 1H), 7.55 (d, J=7.9 Hz, 1H), 7.33 (t, J=6.3 Hz, 1H), 6.02 (q, J=6.5 Hz, 1H), 4.00 (s, 3H), 1.72 (d, J=6.6 Hz, 3H).

### Example 18

### 5-[1-methyl-3-[1-(3-pyridyl)ethoxy]pyrazolo[3,4-c]pyridazin-5-yl]-1H-pyrimidine-2,4-dione

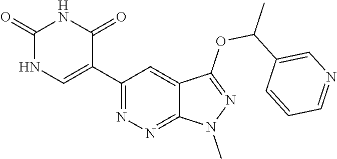 [SMILES: CC(Oc1nn(C)c2nnc(-c3c[nH]c(=O)[nH]c3=O)cc12)c1cccnc1]

5-[1-methyl-3-[1-(3-pyridyl)ethoxy]pyrazolo[3,4-c]pyridazin-5-yl]-1H-pyrimidine-2,4-dione (Example 18) was prepared in analogy to Example 11, by replacing 3-(1-hydroxyethyl)benzonitrile (compound 11.2) with 1-(3-pyridyl)ethanol in step (b). MS: calc'd 366.1 [(M+H)⁺]; measured 366.2 [(M+H)⁺]. ¹H NMR (500 MHz, DMSO-d₆) δ=11.54 (d, J=1.7 Hz, 1H), 11.47-11.42 (m, 1H), 8.79 (s, 1H), 8.63 (s, 1H), 8.56 (d, J=4.7 Hz, 1H), 8.34 (d, J=6.1 Hz, 1H), 8.06 (br d, J=7.8 Hz, 1H), 7.53-7.45 (m, 1H), 6.10 (q, J=6.4 Hz, 1H), 4.02 (s, 3H), 1.75 (d, J=6.6 Hz, 3H).

### Example 19

### 5-[1-methyl-3-[1-(4-pyridyl)ethoxy]pyrazolo[3,4-c]pyridazin-5-yl]-1H-pyrimidine-2,4-dione

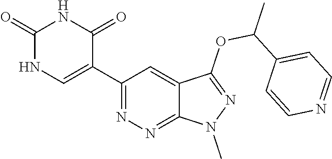 [SMILES: CC(Oc1nn(C)c2nnc(-c3c[nH]c(=O)[nH]c3=O)cc12)c1ccncc1]

5-[1-methyl-3-[1-(4-pyridyl)ethoxy]pyrazolo[3,4-c]pyridazin-5-yl]-1H-pyrimidine-2,4-dione (Example 17) was prepared in analogy to Example 11, by replacing 3-(1-hydroxyethyl)benzonitrile (compound 11.2) with 1-(4-pyridyl)ethanol in step (b). MS: calc'd 366.1 [(M+H)⁺]; measured 366.3 [(M+H)⁺]. ¹H NMR (500 MHz, DMSO-d₆) δ=11.55 (d, J=1.7 Hz, 1H), 11.47 (br d, J=6.1 Hz, 1H), 8.82-8.52 (m, 3H), 8.36 (d, J=6.3 Hz, 1H), 7.89-7.59 (m, 2H), 6.30-5.93 (m, 1H), 4.00 (s, 3H), 1.72 (d, J=6.6 Hz, 3H).

### Example 20

### 5-[1-methyl-3-[1-[3-(trifluoromethyl)phenyl]ethoxy]pyrazolo[3,4-c]pyridazin-5-yl]-1H-pyrimidine-2,4-dione

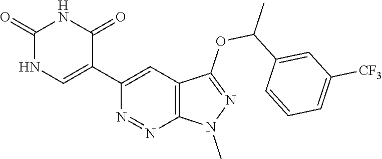 [SMILES: CC(Oc1nn(C)c2nnc(-c3c[nH]c(=O)[nH]c3=O)cc12)c1cccc(C(F)(F)F)c1]

5-[1-methyl-3-[1-[3-(trifluoromethyl)phenyl]ethoxy]pyrazolo[3,4-c]pyridazin-5-yl]-1H-pyrimidine-2,4-dione (Example 20) was prepared in analogy to Example 11, by replacing 3-(1-chloroethyl)benzonitrile (compound 11.3) with 1-(1-bromoethyl)-3-(trifluoromethyl)benzene in step (c). MS: calc'd 433.1 [(M+H)⁺]; measured 433.1 [(M+H)⁺]. ¹H NMR (500 MHz, DMSO-d₆) δ=11.13-10.54 (m, 2H), 8.64 (s, 1H), 8.35 (s, 1H), 7.97-7.81 (m, 2H), 7.67 (s, 1H), 7.63 (d, J=7.6 Hz, 1H), 6.13 (d, J=6.4 Hz, 1H), 4.01 (s, 3H), 1.73 (d, J=6.6 Hz, 3H).

### Example 21

### 5-[1-methyl-3-[1-(2-methylthiazol-4-yl)ethoxy]pyrazolo[3,4-c]pyridazin-5-yl]-1H-pyrimidine-2,4-dione

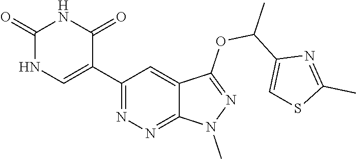 [SMILES: Cc1nc(C(C)Oc2nn(C)c3nnc(-c4c[nH]c(=O)[nH]c4=O)cc23)cs1]

The titled compound was synthesized according to the following scheme

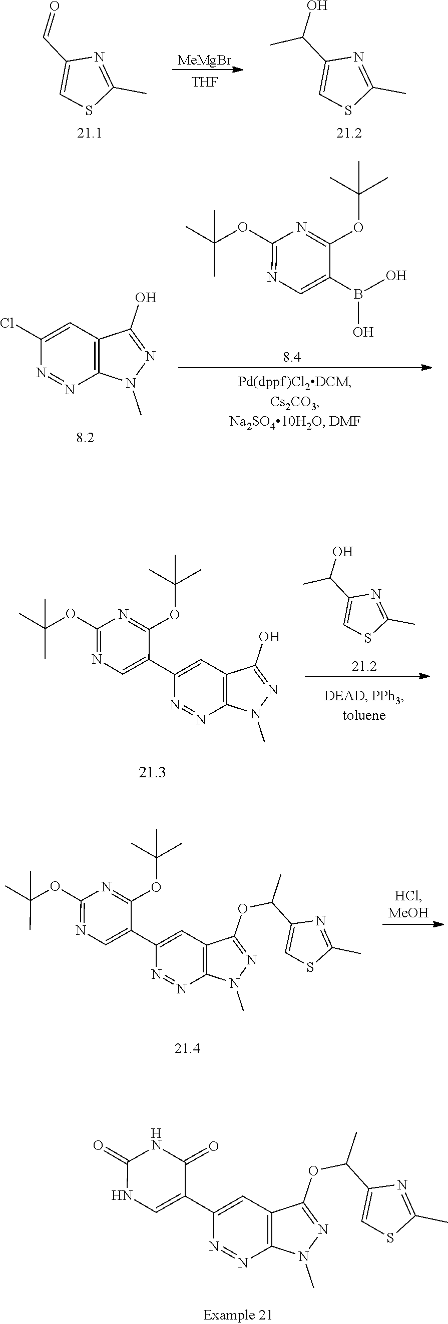

### Step (a): Preparation of 1-(2-methylthiazol-4-yl)ethanol (Compound 21.2)

To a solution of 4-formyl-2-methylthiazole (compound 21.1, 500.0 mg, 3.93 mmol) in THF (5 mL) was added MeMgBr (1M in THF, 6.0 mL, 6.0 mmol) at 0° C. under N₂ atmosphere. The reaction mixture was stirred at 0° C. for 1 h. The mixture was quenched by slow addition of saturated aqueous ammonium chloride (5 mL). Then the mixture was diluted with brine (5 mL) and extracted with EA (10 mL) for three times. The combined organic layer was dried over Na₂SO₄, filtered and concentrated to afford compound 21.2 (500.0 mg). MS: calc'd 144.0 [(M+H)⁺]; measured 126.1 [M−OH]⁺.

### Step (b): Preparation of 5-(2,4-ditert-butoxypyrimidin-5-yl)-1-methyl-pyrazolo[3,4-c]pyridazin-3-ol (Compound 21.3)

To a solution of 5-chloro-1-methyl-pyrazolo[3,4-c]pyridazin-3-ol (compound 8.2, 3.0 g, 16.2 mmol) in DMF (60 mL) was added (2,4-ditert-butoxypyrimidin-5-yl)boronic acid (compound 8.4, 5.2 g, 19.5 mmol), Pd(dppf)Cl₂·DCM (991.0 mg, 1.4 mmol), Na₂SO₄-10H₂O (17.5 g, 54.2 mmol) and Cs₂CO₃ (17.6 g, 54.2 mmol). The resultant mixture was stirred at 85° C. under nitrogen for 16 hr. After being cooled to room temperature, the reaction mixture was diluted with H₂O (150 mL). Then the pH of water phase was adjusted to pH=6 by using saturated aqueous ammonium chloride, and the mixture was extracted with EA (150 mL) for three times. The combined organic layer was washed with brine, dried over Na₂SO₄, filtered and concentrated in vacuo. The residue was purified by silica gel chromatography (50% to 100% EA in PE) to afford compound 21.3 (2.7 g). MS: calc'd 373.2 [(M+H)⁺]; measured 373.2 [(M+H)⁺].

### Step (c): Preparation of 4-[1-[5-(2,4-ditert-butoxypyrimidin-5-yl)-1-methyl-pyrazolo[3,4-c]pyridazin-3-yl]oxyethyl]-2-methyl-thiazole (Compound 21.4)

To a suspension of 5-(2,4-ditert-butoxypyrimidin-5-yl)-1-methyl-pyrazolo[3,4-c]pyridazin-3-ol (compound 21.3, 200.0 mg, 540 μmol), 1-(2-methylthiazol-4-yl)ethanol (compound 21.2, 76.9 mg, 540 μmol) and PPh₃ (281.71 mg, 1.1 mmol) in toluene (8 mL) was added DEAD (187.1 mg, 1.1 mmol). The resultant mixture was stirred at 60° C. for 1 hr. After being cooled to room temperature, the reaction mixture was diluted with H₂O (20 mL), and extracted with EA (20 mL) for three times. The combined organic layer was washed with brine, dried over Na₂SO₄, filtered and concentrated in vacuo. The residue was purified by flash chromatography (silica gel, 24 g, 0% to 30% EA in PE) to afford compound 21.4 (120.0 mg). MS: calc'd 498.2 [(M+H)⁺]; measured 498.4 [(M+H)⁺].

### Step (d): Preparation of 5-[1-methyl-3-[1-(2-methylthiazol-4-yl)ethoxy]pyrazolo[3,4-c]pyridazin-5-yl]-1H-pyrimidine-2,4-dione (Example 21)

To the mixture of 4-[1-[5-(2,4-ditert-butoxypyrimidin-5-yl)-1-methyl-pyrazolo[3,4-c]pyridazin-3-yl]oxyethyl]-2-methyl-thiazole (compound 21.4, 100.0 mg, 0.2 mol) in methanol (3 mL) was added HCl (2.0 M in MeOH, 1.5 mL, 3.0 mmol). The mixture was stirred at room temperature for 2 hr. The reaction mixture was concentrated to afford a crude product, which was purified by pre-HPLC to afford Example 21 (56.0 mg). MS: calc'd 386.1 [(M+H)+]; measured 386.2 [(M+H)+]. ¹H NMR (400 MHz, DMSO-d₆) δ=11.52 (s, 1H), 11.42 (br d, J=5.2 Hz, 1H), 8.57 (s, 1H), 8.37-8.33 (m, 1H), 7.55 (s, 1H), 6.09 (q, J=6.4 Hz, 1H), 4.07 (s, 3H), 2.66 (s, 3H), 1.75 (d, J=6.4 Hz, 3H).

### Example 22

### 5-[1-methyl-3-[1-(5-methylthiazol-2-yl)ethoxy]pyrazolo[3,4-c]pyridazin-5-yl]-1H-pyrimidine-2,4-dione

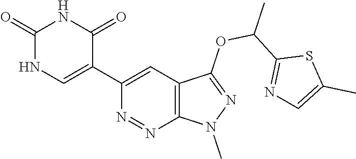 [SMILES: Cc1cnc(C(C)Oc2nn(C)c3nnc(-c4c[nH]c(=O)[nH]c4=O)cc23)s1]

5-[1-methyl-3-[1-(5-methylthiazol-2-yl)ethoxy]pyrazolo[3,4-c]pyridazin-5-yl]-1H-pyrimidine-2,4-dione (Example 22) was prepared in analogy to Example 21, by replacing 1-(2-methylthiazol-4-yl)ethanol (compound 21.2) with 1-(5-methylthiazol-2-yl)ethanol in step (c). MS: calc'd 386.4 [(M+H)+]; measured 386.1 [(M+H)+]. ¹H NMR (400 MHz, DMSO-d₆) δ=11.52 (s, 1H), 11.43 (br d, J=4.9 Hz, 1H), 8.60 (s, 1H), 8.36 (d, J=6.1 Hz, 1H), 7.48 (s, 1H), 6.22 (q, J=6.5 Hz, 1H), 4.07 (s, 3H), 2.41 (s, 3H), 1.80 (d, J=6.5 Hz, 3H).

### Example 23

### 5-[3-[1-(4-chloro-1-methyl-pyrazol-3-yl)ethoxy]-1-methyl-pyrazolo[3,4-c]pyridazin-5-yl]-1H-pyrimidine-2,4-dione

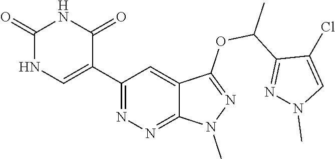 [SMILES: CC(Oc1nn(C)c2nnc(-c3c[nH]c(=O)[nH]c3=O)cc12)c1nn(C)cc1Cl]

5-[3-[1-(4-chloro-1-methyl-pyrazol-3-yl)ethoxy]-1-methyl-pyrazolo[3,4-c]pyridazin-5-yl]-1H-pyrimidine-2,4-dione (Example 23) was prepared in analogy to Example 21, by replacing 4-formyl-2-methylthiazole (compound 21.1) with 4-chloro-1-methylpyrazole-3-carboxaldehyde in step (a). MS: calc'd 403.1, 405.1 [(M+H)⁺]; measured 403.2, 405.2 [(M+H)⁺]. ¹H NMR (400 MHz, DMSO-d₆) δ=11.50 (s, 1H), 11.43 (br d, J=5.6 Hz, 1H), 8.53 (s, 1H), 8.34 (d, J=6.4 Hz, 1H), 7.92 (s, 1H), 6.03 (q, J=6.4 Hz, 1H), 4.06 (s, 3H), 3.81 (s, 3H), 1.75 (d, J=6.4 Hz, 3H).

### Example 24

### 5-[1-methyl-3-[1-(2-methylpyrazol-3-yl)ethoxy]pyrazolo[3,4-c]pyridazin-5-yl]-1H-pyrimidine-2,4-dione

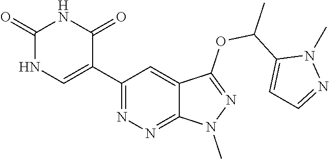 [SMILES: CC(Oc1nn(C)c2nnc(-c3c[nH]c(=O)[nH]c3=O)cc12)c1ccnn1C]

5-[1-methyl-3-[1-(2-methylpyrazol-3-yl)ethoxy]pyrazolo[3,4-c]pyridazin-5-yl]-1H-pyrimidine-2,4-dione (Example 24) was prepared in analogy to Example 11, by replacing 3-(1-hydroxyethyl)benzonitrile (compound 11.2) with 1-(2-methylpyrazol-3-yl)ethanol in step (b). MS: calc'd 369.1 [(M+H)⁺], measured 369.2 [(M+H)⁺]. ¹H NMR (400 MHz, DMSO-d₆) δ=11.51 (s, 1H), 11.45-11.35 (m, 1H), 8.55 (s, 1H), 8.37-8.29 (m, 1H), 7.37 (d, J=2.0 Hz, 1H), 6.45 (d, J=2.0 Hz, 1H), 6.16 (q, J=6.4 Hz, 1H), 4.08 (s, 3H), 3.87 (s, 3H), 1.77 (d, J=6.4 Hz, 3H).

### Example 25

### 5-[3-[1-(1,3-benzoxazol-2-yl)ethoxy]-1-methyl-pyrazolo[3,4-c]pyridazin-5-yl]-1H-pyrimidine-2,4-dione

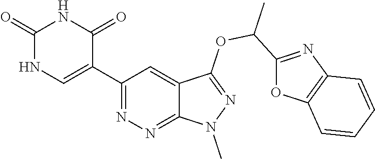 [SMILES: CC(Oc1nn(C)c2nnc(-c3c[nH]c(=O)[nH]c3=O)cc12)c1nc2ccccc2o1]

5-[1-methyl-3-[1-(2-methylpyrazol-3-yl)ethoxy]pyrazolo[3,4-c]pyridazin-5-yl]-1H-pyrimidine-2,4-dione (Example 24) was prepared in analogy to Example 11, by replacing 3-(1-hydroxyethyl)benzonitrile (compound 11.2) with 1-(1,3-benzoxazol-2-yl)ethanol in step (b). MS: calc'd 406.1 [(M+H)⁺], measured 406.2 [(M+H)⁺]. ¹H NMR (400 MHz, DMSO-d₆) δ=11.53 (s, 1H), 11.45 (br d, J=6.0 Hz, 1H), 8.63 (s, 1H), 8.35 (d, J=6.0 Hz, 1H), 7.79-7.72 (m, 2H), 7.47-7.37 (m, 2H), 6.30 (q, J=6.8 Hz, 1H), 4.03 (s, 3H), 1.90 (d, J=6.4 Hz, 3H).

### Example 26

### 5-[1-methyl-3-[(1S)-1-(2-pyridyl)ethoxy]pyrazolo[3,4-c]pyridazin-5-yl]-1H-pyrimidine-2,4-dione

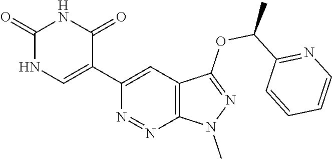 [SMILES: C[C@H](Oc1nn(C)c2nnc(-c3c[nH]c(=O)[nH]c3=O)cc12)c1ccccn1]

The titled compound was synthesized according to the following scheme

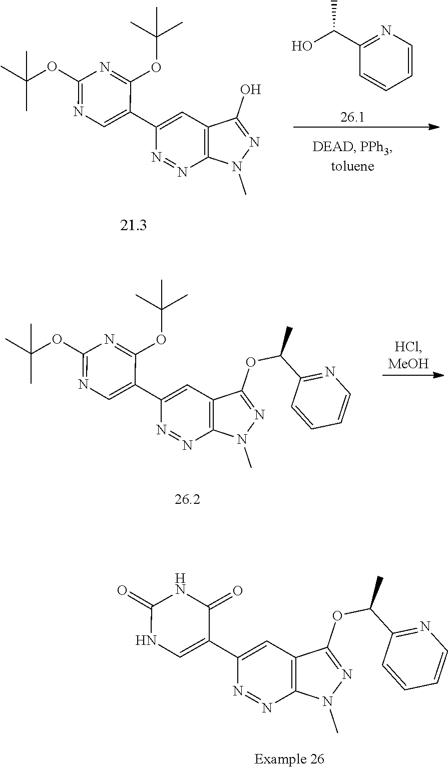

### Step (a): Preparation of 5-(2,4-ditert-butoxypyrimidin-5-yl)-1-methyl-3-[(1S)-1-(2-pyridyl)ethoxy]pyrazolo[3,4-c]pyridazine (Compound 26.2)

To a solution of 5-(2,4-ditert-butoxypyrimidin-5-yl)-1-methyl-pyrazolo[3,4-c]pyridazin-3-ol (compound 21.3, 300.0 mg, 805.5 μmol) in toluene (8 mL) was added (1R)-1-(2-pyridyl)ethanol (compound 26.1, 148.8 mg, 1.2 mmol), PPh₃ (316.9 mg, 1.2 mmol) and DEAD (220 mg, 200 μL, 1.3 mmol). The resultant mixture was stirred at 60° C. for 1 hr. After being cooled to room temperature, the reaction mixture was diluted with H₂O (20 mL), and extracted with EA (20 mL) for three times. The combined organic layer was washed with brine, dried over Na₂SO₄, filtered and concentrated in vacuo. The residue was purified by flash chromatography (silica gel, 24 g, 0% to 30% EA in PE) to afford compound 26.2 (338.6 mg). MS: calc'd 478.3 [(M+H)⁺]; measured 478.3 [(M+H)⁺].

### Step (b): Preparation of 5-[1-methyl-3-[(1S)-1-(2-pyridyl)ethoxy]pyrazolo[3,4-c]pyridazin-5-yl]-1H-pyrimidine-2,4-dione (Example 26)

To the mixture of 5-(2,4-ditert-butoxypyrimidin-5-yl)-1-methyl-3-[(1S)-1-(2-pyridyl)ethoxy]pyrazolo[3,4-c]pyridazine (compound 26.2, 338.6 mg, 709.0 μmol) in methanol (3 mL) was added HCl (2.0 M in MeOH, 1.5 mL, 3.0 mmol). The mixture was stirred at 22° C. for 2 hr. The reaction mixture was concentrated to afford a crude product, which was purified by pre-HPLC to afford Example 26 (229.0 mg). MS: calc'd 366.1 [(M+H)⁺]; measured 366.1 [(M+H)⁺]. ¹H NMR (400 MHz, DMSO-d₆) δ=11.53 (s, 1H), 11.47 (br d, J=4.3 Hz, 1H), 8.67 (s, 2H), 8.36 (d, J=5.8 Hz, 1H), 8.02 (d, J=5.3 Hz, 1H), 7.74 (s, 1H), 7.51 (d, J=3.0 Hz, 1H), 6.17-6.02 (m, 1H), 4.00 (s, 3H), 1.76 (d, J=6.1 Hz, 3H).

### Example 27

### 5-[1-methyl-3-[(1S)-1-phenylethoxy]pyrazolo[3,4-c]pyridazin-5-yl]-1H-pyrimidine-2,4-dione

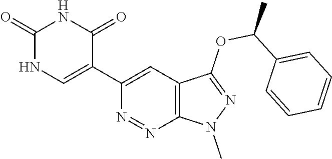 [SMILES: C[C@H](Oc1nn(C)c2nnc(-c3c[nH]c(=O)[nH]c3=O)cc12)c1ccccc1]

5-[1-methyl-3-[(1S)-1-phenylethoxy]pyrazolo[3,4-c]pyridazin-5-yl]-1H-pyrimidine-2,4-dione (Example 27) was prepared in analogy to Example 26, by replacing (1R)-1-(2-pyridyl)ethanol (compound 26.2) with (1R)-1-phenylethanol in step (a). MS: calc'd 365.1 [(M+H)⁺], measured 365.2 [(M+H)⁺]. ¹H NMR (400 MHz, DMSO-d₆) δ=11.51 (s, 1H), 11.42 (br s, 1H), 8.61 (s, 1H), 8.33 (s, 1H), 7.51 (d, J=7.3 Hz, 2H), 7.44-7.34 (m, 2H), 7.33-7.25 (m, 1H), 6.02 (q, J=6.5 Hz, 1H), 4.02 (s, 3H), 1.70 (d, J=6.4 Hz, 3H).

### Example 28

### 5-[3-[(1R)-2,2-difluoro-1-phenyl-ethoxy]-1-methyl-pyrazolo[3,4-c]pyridazin-5-yl]-1H-pyrimidine-2,4-dione

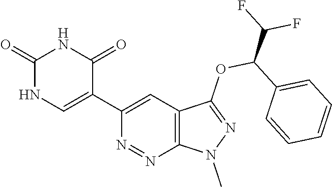 [SMILES: Cn1nc(O[C@H](c2ccccc2)C(F)F)c2cc(-c3c[nH]c(=O)[nH]c3=O)nnc21]

The titled compound was synthesized according to the following scheme

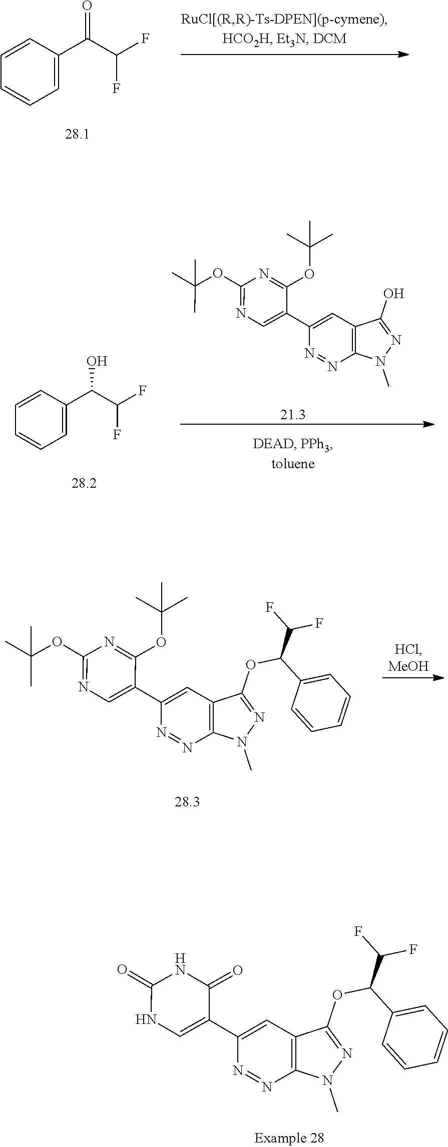

### Step (a): Preparation of (1S)-2,2-difluoro-1-phenyl-ethanol (Compound 28.2)

To a solution of 2,2-difluoro-1-phenyl-ethanone (compound 28.1, 354.0 mg, 300 μL, 2.3 mmol) in DCM (2 mL) was added HCOOH (1.1 g, 900 μL, 23.5 mmol), Et₃N (943.8 mg, 1.3 mL, 9.3 mmol) and RuCl[(R,R)-TsDPEN](p-cymene) (14.4 mg, 22.7 μmol). The resultant mixture was stirred at room temperature under nitrogen for 24 hr. The reaction mixture was diluted with saturated NaHCO₃ (20 mL), and extracted with DCM (20 mL) for three times. The combined organic layer was washed with brine, dried over Na₂SO₄, filtered and concentrated in vacuo. The residue was purified by flash chromatography (silica gel, 12 g, 0% to 50% EA in PE) to afford compound 28.2 (302.0 mg). MS: calc'd 141.0 [(M−OH)⁺]; measured 141.0 [(M−OH)⁺].

### Step (b): Preparation of 5-(2,4-ditert-butoxypyrimidin-5-yl)-1-methyl-3-[(1R)-2,2-difluoro-1-phenyl-ethoxy]pyrazolo[3,4-c]pyridazine (Compound 28.3)

To a solution of 5-(2,4-ditert-butoxypyrimidin-5-yl)-1-methyl-pyrazolo[3,4-c]pyridazin-3-ol (compound 21.3, 180.0 mg, 483.3 μmol) in toluene (4 mL) was added (1S)-2,2-difluoro-1-phenyl-ethanol (compound 28.2, 130.1 mg, 773.3 μmol), PPh₃ (202.8 mg, 773.3 μmol) and DEAD (143.0 mg, 130 μL, 821.1 μmol). The resultant mixture was stirred at 60° C. for 3 hr. After being cooled to room temperature, the reaction mixture was diluted with H₂O (20 mL), and extracted with EA (20 mL) for three times. The combined organic layer was washed with brine, dried over Na₂SO₄, filtered and concentrated in vacuo. The residue was purified by flash chromatography (silica gel, 24 g, 0% to 25% EA in PE) to afford compound 28.3 (219.4 mg). MS: calc'd 513.2 [(M+H)⁺]; measured 513.3 [(M+H)⁺].

### Step (c): Preparation of 5-[3-[(1R)-2,2-difluoro-1-phenyl-ethoxy]-1-methyl-pyrazolo[3,4-c]pyridazin-5-yl]-1H-pyrimidine-2,4-dione (Example 28)

To the mixture of 5-(2,4-ditert-butoxypyrimidin-5-yl)-1-methyl-3-[(1R)-2,2-difluoro-1-phenyl-ethoxy]pyrazolo[3,4-c]pyridazine (compound 28.3, 219.4 mg, 428.1 μmol) in methanol (1.5 mL) was added HCl (2.0 M in MeOH, 1.0 mL, 2.0 mmol). The mixture was stirred at 22° C. for 2 hr. The reaction mixture was concentrated to afford a crude product, which was purified by pre-HPLC to afford Example 28 (137.4 mg). MS: calc'd 401.1 [(M+H)⁺]; measured 401.0 [(M+H)⁺]. ¹H NMR (500 MHz, DMSO-d₆) δ=11.55 (s, 1H), 11.44 (br s, 1H), 8.69 (s, 1H), 8.36 (s, 1H), 7.63-7.57 (m, 2H), 7.47-7.37 (m, 3H), 6.58 (td, J=54.5, 3.5 Hz, 1H), 6.19 (td, J=11.4, 3.1 Hz, 1H), 4.02 (s, 3H).

### Example 29A and 29B

### 5-[1-methyl-3-[(1R)-2,2,2-trifluoro-1-(2-pyridyl)ethoxy]pyrazolo[3,4-c]pyridazin-5-yl]-1H-pyrimidine-2,4-dione and 5-[1-methyl-3-[(1S)-2,2,2-trifluoro-1-(2-pyridyl)ethoxy]pyrazolo[3,4-c]pyridazin-5-yl]-1H-pyrimidine-2,4-dione

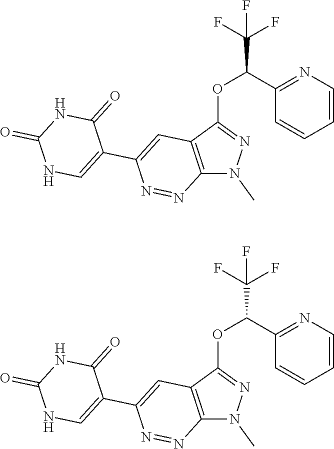 [SMILES: Cn1nc(O[C@H](c2ccccn2)C(F)(F)F)c2cc(-c3c[nH]c(=O)[nH]c3=O)nnc21 ; Cn1nc(O[C@@H](c2ccccn2)C(F)(F)F)c2cc(-c3c[nH]c(=O)[nH]c3=O)nnc21]

The titled compound was synthesized according to the following scheme:

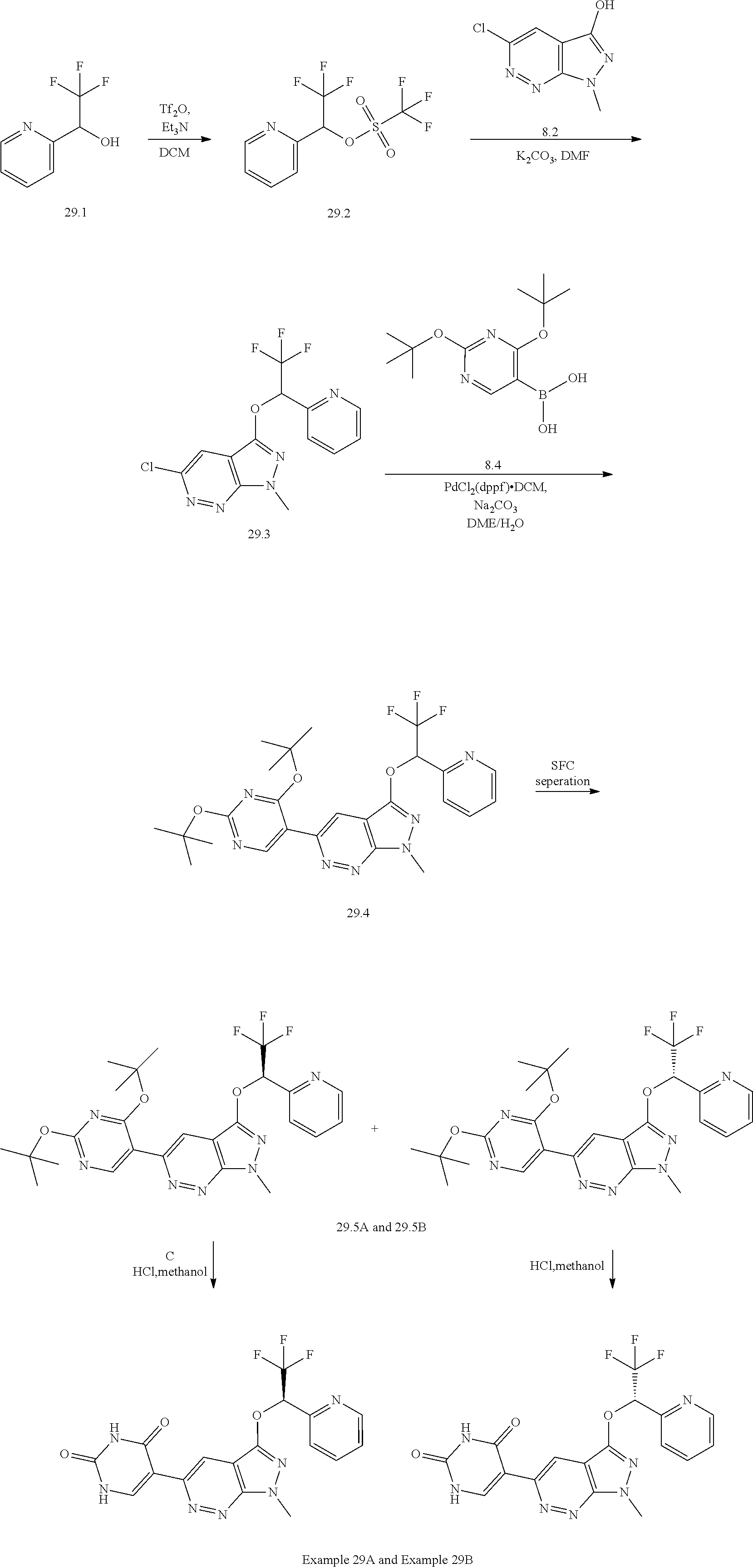

### Step (a): Preparation of [2,2,2-trifluoro-1-(2-pyridyl)ethyl]trifluoromethanesulfonate (Compound 29.2)

To the mixture of 2,2,2-trifluoro-1-(2-pyridyl)ethanol (compound 29.1, 1.8 g, 10.2 mmol), triethylamine (1.54 g, 2.1 mL, 15.2 mmol) in DCM (20 mL) was added dropwise trifluoromethanesulfonic anhydride (3.15 g, 1.9 mL, 11.2 mmol) at 0° C. The mixture was stirred at 0° C. for 10 minutes. The red mixture with compound 29.2 was added to the subsequent step directly without work up.

### Step (b): Preparation of 5-chloro-1-methyl-3-[2,2,2-trifluoro-1-(2-pyridyl)ethoxy]pyrazolo[3,4-c]pyridazine (Compound 29.3)

To the mixture of 5-chloro-1-methyl-pyrazolo[3,4-c]pyridazin-3-ol (compound 8.2, 1.5 g, 8.1 mmol) in DMF (15 mL) was added potassium carbonate (1.68 g, 12.2 mmol) and [2,2,2-trifluoro-1-(2-pyridyl)ethyl]trifluoromethanesulfonate (compound 29.2, crude product from the previous step) was subsequently added at 0° C. to the above mixture. The mixture was stirred at 60° C. for 3 hr. After being cooled to room temperature, the reaction was quenched with H₂O (15 mL) and the mixture was extracted with EA (60 mL) for three times. The combined organic layer was washed with brine, dried over Na₂SO₄ and concentrated in vacuo. The residue was purified by silica gel column chromatography (eluent: PE/EA, from 100/1 to 3/1) to afford compound 29.3 (1.12 g). MS: calc'd 344.7 [(M+H)⁺], measured 344.0 [(M+H)⁺].

### Step (c): Preparation of 5-(2,4-ditert-butoxypyrimidin-5-yl)-1-methyl-3-[2,2,2-trifluoro-1-(2-pyridyl)ethoxy]pyrazolo[3,4-c]pyridazine (Compound 29.4)

To the mixture of (2,4-ditert-butoxypyrimidin-5-yl)boronic acid (539.1 mg, 2.0 mmol) and 5-chloro-1-methyl-3-[2,2,2-trifluoro-1-(2-pyridyl)ethoxy]pyrazolo[3,4-c]pyridazine (compound 29.3, 530 mg, 1.6 mmol) in 1,2-dimethoxyethane (8 mL) and water (2 mL) were added Pd(dppf)Cl₂·DCM (126.3 mg, 154.7 μmol) and Na₂CO₃ (655.7 mg, 6.2 mmol). The mixture was stirred at 90° C. under nitrogen for 1 hr. After being cooled to room temperature, The mixture was diluted with water (30 mL), and extracted with EA (10 mL) twice. The combined organic layer was dried over anhydrous Na₂SO₄ and concentrated in vacuo. The residue was purified by silica gel column chromatography (eluent: PE/EA, from 100/1 to 3/1) to afford compound 29.4 (900 mg). MS: calc'd 532.5 [(M+H)⁺], measured 532.2 [(M+H)⁺].

### Step (d): Preparation of 5-(2,4-ditert-butoxypyrimidin-5-yl)-1-methyl-3-[(1R)-2,2,2-trifluoro-1-(2-pyridyl)ethoxy]pyrazolo[3,4-c]pyridazine and 5-(2,4-ditert-butoxypyrimidin-5-yl)-1-methyl-3-[(1S)-2,2,2-trifluoro-1-(2-pyridyl)ethoxy]pyrazolo[3,4-c]pyridazine (Compound 29.5A and Compound 29.5B)

Compound 30.4 (900 mg) was resolved by SFC to give two single isomers: compound 29.5A (faster eluted, 400 mg) MS: calc'd 532.5 (M+H)⁺, measured 532.2 (M+H)⁺; and compound 29.5B (slower eluted, 330 mg) MS: calc'd 532.5 (M+H)⁺, measured 532.2 (M+H)⁺, with SFC 150 Mgm Column: (s,s)Whelk-O1 250×30 mm I.D., 5 μm. Mobile phase: A for CO₂ and B for IPA (0.1% NH₃H₂O) Gradient: B 40% Flow rate: 80 mL/min Back pressure: 100 bar.

### Step (e): Preparation of 5-[1-methyl-3-[(1R)-2,2,2-trifluoro-1-(2-pyridyl)ethoxy]pyrazolo[3,4-c]pyridazin-5-yl]-1H-pyrimidine-2,4-dione and 5-[1-methyl-3-[(1S)-2,2,2-trifluoro-1-(2-pyridyl)ethoxy]pyrazolo[3,4-c]pyridazin-5-yl]-1H-pyrimidine-2,4-dione (Example 29A and 29B)

To the mixture of compound 29.5A (400 mg, 752.5 μmol) in methanol (4 mL) was added 2 M HCl (1.3 mL, 2.7 mmol). The mixture was stirred at room temperature for 1 hr, then filtered and washed with ice methanol (1 mL) twice to afford Example 29A (236.1 mg). MS: calc'd 420.3 [(M+H)⁺]; measured 420.0 [(M+H)⁺]. ¹H NMR (400 MHz, DMSO-d₆) δ=11.56 (s, 1H), 11.46 (br d, J=6.0 Hz, 1H), 8.71 (s, 1H), 8.67 (d, J=4.9 Hz, 1H), 8.37 (d, J=6.1 Hz, 1H), 7.93 (dt, J=7.7, 1.7 Hz, 1H), 7.79 (d, J=7.9 Hz, 1H), 7.50 (ddd, J=7.5, 4.9, 0.9 Hz, 1H), 6.59 (q, J=6.7 Hz, 1H), 4.01 (s, 3H).

To the mixture of compound 29.5B (330 mg, 620.9 μmol) in methanol (1.5 mL) was added 2 M HCl (1.6 mL, 3.1 mmol). The mixture was stirred at room temperature for 1 hr, then filtered and washed with ice methanol (1 mL) twice to afford Example 29B (179.9 mg). MS: calc'd 420.3 [(M+H)+]; measured 420.1 [(M+H)+]. ¹H NMR (400 MHz, DMSO-d₆) δ=11.55 (s, 1H), 11.45 (br d, J=5.9 Hz, 1H), 8.71 (s, 1H), 8.66 (d, J=4.3 Hz, 1H), 8.37 (d, J=6.3 Hz, 1H), 7.92 (dt, J=7.8, 1.6 Hz, 1H), 7.79 (d, J=7.9 Hz, 1H), 7.50 (t, J=6.1 Hz, 1H), 6.59 (q, J=6.7 Hz, 1H), 4.01 (s, 3H).

### Example 30A and 30B

### 5-[1-methyl-3-[(1R)-2,2,2-trifluoro-1-phenyl-ethoxy]pyrazolo[3,4-c]pyridazin-5-yl]-1H-pyrimidine-2,4-dione and 5-[1-methyl-3-[(1S)-2,2,2-trifluoro-1-phenyl-ethoxy]pyrazolo[3,4-c]pyridazin-5-yl]-1H-pyrimidine-2,4-dione

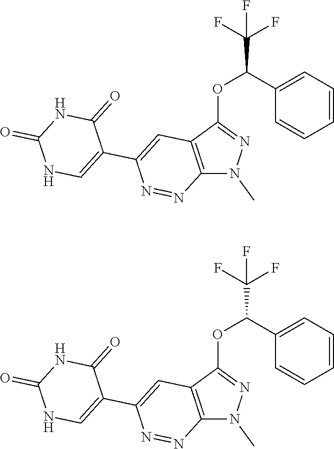 [SMILES: Cn1nc(O[C@H](c2ccccc2)C(F)(F)F)c2cc(-c3c[nH]c(=O)[nH]c3=O)nnc21 ; Cn1nc(O[C@@H](c2ccccc2)C(F)(F)F)c2cc(-c3c[nH]c(=O)[nH]c3=O)nnc21]

Example 30A and 30B was prepared in analogy to Example 29A and 29B, by replacing 2,2,2-trifluoro-1-(2-pyridyl)ethanol (compound 29.1) with 2,2,2-trifluoro-1-phenyl-ethanol in step (a). Example 30A MS: calc'd 419.3 [(M+H)+]; measured 419.1 [(M+H)+]. ¹H NMR (400 MHz, DMSO-d₆) δ=11.55 (s, 1H), 11.45 (br d, J=5.1 Hz, 1H), 8.70 (s, 1H), 8.36 (d, J=6.0 Hz, 1H), 7.74-7.64 (m, 2H), 7.50-7.43 (m, 3H), 6.67 (q, J=6.7 Hz, 1H), 4.03 (s, 3H). Example 30B MS: calc'd 419.3 [(M+H)+]; measured 419.1 [(M+H)+]. ¹H NMR (400 MHz, DMSO-d₆) δ=11.55 (s, 1H), 11.45 (br d, J=5.1 Hz, 1H), 8.70 (s, 1H), 8.36 (d, J=6.1 Hz, 1H), 7.72-7.66 (m, 2H), 7.50-7.43 (m, 3H), 6.67 (q, J=6.6 Hz, 1H), 4.03 (s, 3H).

### Example 31A and 31B

### 5-[3-[(1R)-2,2-difluoro-1-(2-pyridyl)ethoxy]-1-methyl-pyrazolo[3,4-c]pyridazin-5-yl]-1H-pyrimidine-2,4-dione and 5-[3-[(1S)-2,2-difluoro-1-(2-pyridyl)ethoxy]-1-methyl-pyrazolo[3,4-c]pyridazin-5-yl]-1H-pyrimidine-2,4-dione

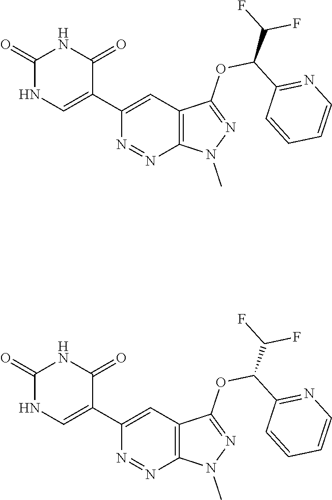 [SMILES: Cn1nc(O[C@H](c2ccccn2)C(F)F)c2cc(-c3c[nH]c(=O)[nH]c3=O)nnc21 ; Cn1nc(O[C@@H](c2ccccn2)C(F)F)c2cc(-c3c[nH]c(=O)[nH]c3=O)nnc21]

The titled compound was synthesized according to the following scheme:

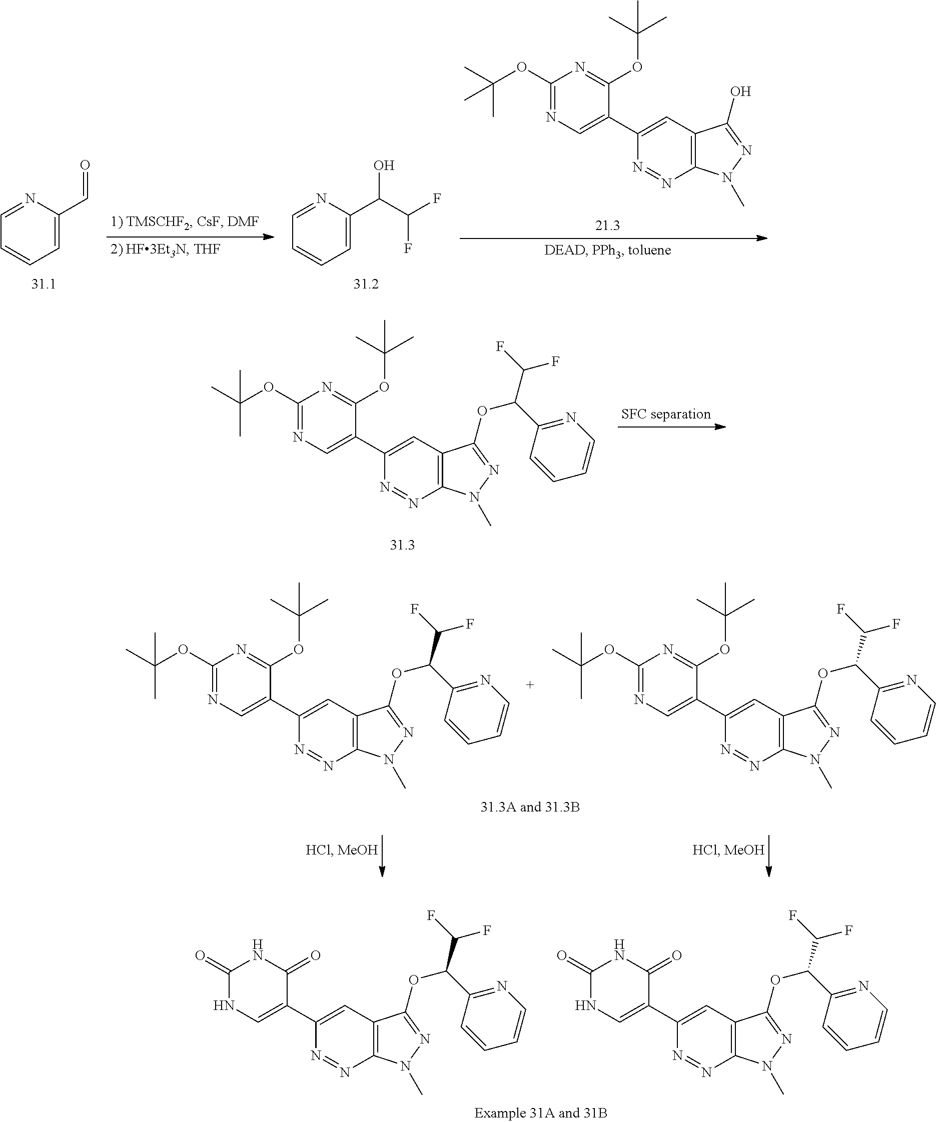

### Step (a): Preparation of 2,2-difluoro-1-(2-pyridyl)ethanol (Compound 31.2)

To a solution of pyridine-2-carbaldehyde (compound 31.1, 1.7 g, 1.5 mL, 15.7 mmol) in DMF (20 mL) was added TMSCHF₂ (2.6 g, 3.0 mL, 21.2 mmol) and CsF (238.3 mg, 1.6 mmol). The resultant mixture was stirred at room temperature under nitrogen for 2 hr, then diluted with saturated NaCl (100 mL), and extracted with EA (100 mL) for three times. The combined organic layer was washed with brine, dried over Na₂SO₄, filtered and concentrated in vacuo. The residue was dissolved in THF (20 mL). To the resultant solution was added HF-3Et₃N (4.0 g, 4.0 mL, 24.5 mmol) at 0° C. After being stirred at 0° C. for 1 hr, the reaction was quenched by saturated NaHCO₃ (100 mL), and the mixture was extracted with EA (100 mL) for three times. The combined organic layer was washed with brine, dried over Na₂SO₄, filtered and concentrated in vacuo. The residue was purified by flash chromatography (silica gel, 80 g, 0% to 50% EA in PE) to afford compound 31.2 (1.77 g). MS: calc'd 160.1 [(M+H)⁺]; measured 160.0 [(M+H)⁺].

### Step (b): Preparation of 5-(2,4-ditert-butoxypyrimidin-5-yl)-3-[2,2-difluoro-1-(2-pyridyl)ethoxy]-1-methyl-pyrazolo[3,4-c]pyridazine (Compound 31.3)

To a solution of 5-(2,4-ditert-butoxypyrimidin-5-yl)-1-methyl-pyrazolo[3,4-c]pyridazin-3-ol (compound 21.3, 500.0 mg, 1.3 mmol) in toluene (13 mL) was added 2,2-difluoro-1-(2-pyridyl)ethanol (compound 31.2, 277.7 mg, 1.7 mmol), PPh₃ (493.0 mg, 1.9 mmol) and DEAD (330.0 mg, 300 μL, 1.9 mmol). The resultant mixture was stirred at 60° C. for 1 hr. After being cooled to room temperature, the reaction mixture was diluted with H₂O (40 mL), and extracted with EA (40 mL) for three times. The combined organic layer was washed with brine, dried over Na₂SO₄, filtered and concentrated in vacuo. The residue was purified by flash chromatography (silica gel, 30 g, 0% to 30% EA in PE) to afford compound 31.3 (582.0 mg). MS: calc'd 514.2 [(M+H)⁺]; measured 514.3 [(M+H)⁺].

### Step (c): Preparation of 5-(2,4-ditert-butoxypyrimidin-5-yl)-1-methyl-3-[(1R)-2,2-difluoro-1-(2-pyridyl)ethoxy]pyrazolo[3,4-c]pyridazine and 5-(2,4-ditert-butoxypyrimidin-5-yl)-1-methyl-3-[(1S)-2,2-difluoro-1-(2-pyridyl)ethoxy]pyrazolo[3,4-c]pyridazine (Compound 31.3A and 31.3B)

Compound 31.3 (582.0 mg) was resolved by SFC to give two single isomers: compound 31.3A (faster eluted, 251.4 mg) MS: calc'd 514.2 [(M+H)+], measured 514.3 [(M+H)+]; and compound 31.3B (slower eluted, 238.3 mg) MS: calc'd 514.2 [(M+H)⁺], measured 514.3 [(M+H)⁺], with SFC 150 Mgm, Column: (s,s)Whelk-O1 250×30 mm I.D. 5 μm, mobile phase: A for CO₂ and B for MeOH (0.1% NH₃·H₂O), gradient: B 30%, flow rate: 80 mL/min, back pressure: 100 bar.

### Step (d): 5-[3-[(1R)-2,2-difluoro-1-(2-pyridyl)ethoxy]-1-methyl-pyrazolo[3,4-c]pyridazin-5-yl]-1H-pyrimidine-2,4-dione and 5-[3-[(1S)-2,2-difluoro-1-(2-pyridyl)ethoxy]-1-methyl-pyrazolo[3,4-c]pyridazin-5-yl]-1H-pyrimidine-2,4-dione (Example 31A and 31B)

To the mixture of compound 31.3A (251.4 mg, 489.5 μmol) in methanol (2 mL) was added HCl (2.0 M in MeOH, 400 μL, 800 μmol). The mixture was stirred at room temperature for 2 hr. The reaction mixture was concentrated to afford a crude product, which was purified by pre-HPLC to afford Example 31A (92.6 mg). MS: calc'd 402.1 [(M+H)⁺]; measured 402.0 [(M+H)⁺]. ¹H NMR (400 MHz, DMSO-d₆) δ=11.55 (s, 1H), 11.45 (br d, J=5.1 Hz, 1H), 8.70 (s, 1H), 8.65 (br d, J=4.3 Hz, 1H), 8.38 (d, J=6.3 Hz, 1H), 7.88 (td, J=7.7, 1.6 Hz, 1H), 7.67 (d, J=7.9 Hz, 1H), 7.44 (dd, J=6.9, 5.1 Hz, 1H), 6.72 (td, J=53.8, 3.0 Hz, 1H), 6.29-6.11 (m, 1H), 4.02 (s, 3H).

To the mixture of compound 31.3B (238.3 mg, 464.2 μmol) in methanol (2 mL) was added HCl (2.0 M in MeOH, 400 μL, 800 μmol). The mixture was stirred at room temperature for 2 hr. The reaction mixture was concentrated to afford a crude product, which was purified by pre-HPLC to afford Example 31B (85.0 mg). MS: calc'd 402.1 [(M+H)⁺]; measured 402.0 [(M+H)⁺]. ¹H NMR (400 MHz, DMSO-d₆) δ=11.55 (s, 1H), 11.46 (br d, J=5.8 Hz, 1H), 8.70 (s, 1H), 8.65 (dd, J=4.8, 0.6 Hz, 1H), 8.38 (d, J=6.1 Hz, 1H), 7.89 (td, J=7.7, 1.7 Hz, 1H), 7.67 (d, J=7.8 Hz, 1H), 7.45 (ddd, J=7.5, 4.8, 0.9 Hz, 1H), 6.72 (td, J=53.8, 3.4 Hz, 1H), 6.22 (ddd, J=14.1, 8.8, 3.3 Hz, 1H), 4.02 (s, 3H).

### Example 32A and 32B

### 5-[3-[(1R)-2,2-difluoro-1-(5-fluoro-2-pyridyl)ethoxy]-1-methyl-pyrazolo[3,4-c]pyridazin-5-yl]-1H-pyrimidine-2,4-dione and 5-[3-[(1S)-2,2-difluoro-1-(5-fluoro-2-pyridyl)ethoxy]-1-methyl-pyrazolo[3,4-c]pyridazin-5-yl]-1H-pyrimidine-2,4-dione

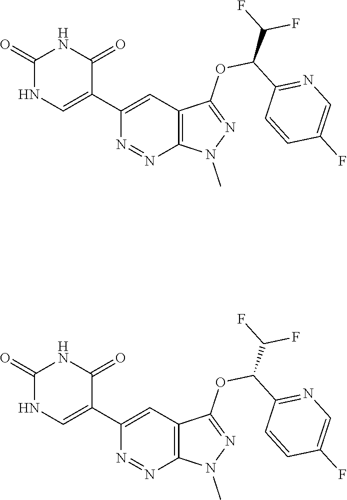 [SMILES: Cn1nc(O[C@H](c2ccc(F)cn2)C(F)F)c2cc(-c3c[nH]c(=O)[nH]c3=O)nnc21 ; Cn1nc(O[C@@H](c2ccc(F)cn2)C(F)F)c2cc(-c3c[nH]c(=O)[nH]c3=O)nnc21]

Example 32A and 32B was prepared in analogy to Example 31A and 31B, by replacing pyridine-2-carbaldehyde (compound 31.1) with 5-fluoropyridine-2-carbaldehyde in step (a). Example 32A MS: calc'd 420.1 [(M+H)⁺]; measured 420.0 [(M+H)⁺]. ¹H NMR (400 MHz, DMSO-d₆) δ=11.54 (s, 1H), 11.45 (br d, J=5.8 Hz, 1H), 8.68 (s, 1H), 8.66 (d, J=2.4 Hz, 1H), 8.37 (d, J=6.1 Hz, 1H), 7.88-7.73 (m, 2H), 6.70 (td, J=53.9, 3.4 Hz, 1H), 6.34-6.16 (m, 1H), 4.01 (s, 3H). Example 32B MS: calc'd 420.1 [(M+H)⁺]; measured 420.1 [(M+H)⁺]. ¹H NMR (400 MHz, DMSO-d₆) δ=11.54 (s, 1H), 11.45 (br d, J=5.9 Hz, 1H), 8.69 (s, 1H), 8.66 (d, J=2.3 Hz, 1H), 8.37 (d, J=6.1 Hz, 1H), 7.88-7.74 (m, 2H), 6.71 (td, J=53.9, 3.3 Hz, 1H), 6.31-6.19 (m, 1H), 4.02 (s, 3H).

### Example 33A and 33B

### 5-[3-[(1R)-2,2-difluoro-1-(6-methyl-2-pyridyl)ethoxy]-1-methyl-pyrazolo[3,4-c]pyridazin-5-yl]-1H-pyrimidine-2,4-dione and 5-[3-[(1S)-2,2-difluoro-1-(6-methyl-2-pyridyl)ethoxy]-1-methyl-pyrazolo[3,4-c]pyridazin-5-yl]-1H-pyrimidine-2,4-dione

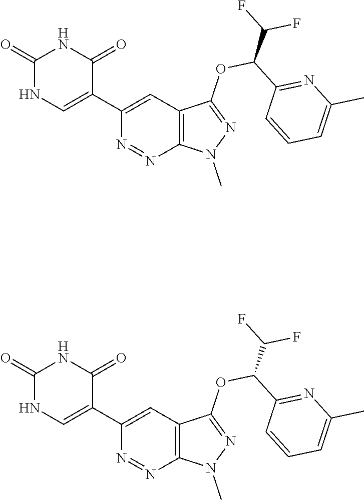 [SMILES: Cc1cccc([C@@H](Oc2nn(C)c3nnc(-c4c[nH]c(=O)[nH]c4=O)cc23)C(F)F)n1 ; Cc1cccc([C@H](Oc2nn(C)c3nnc(-c4c[nH]c(=O)[nH]c4=O)cc23)C(F)F)n1]

Example 33A and 33B were prepared in analogy to Example 31A and 31B, by replacing pyridine-2-carbaldehyde (compound 31.1) with 6-methylpyridine-2-carbaldehyde in step (a). Example 33A MS: calc'd 416.1 [(M+H)⁺]; measured 416.1 [(M+H)⁺]. ¹H NMR (400 MHz, DMSO-d₆) δ=11.54 (s, 1H), 11.44 (br d, J=5.8 Hz, 1H), 8.70 (s, 1H), 8.37 (d, J=6.3 Hz, 1H), 7.75 (t, J=7.8 Hz, 1H), 7.44 (d, J=7.6 Hz, 1H), 7.29 (d, J=7.8 Hz, 1H), 6.68 (td, J=53.7, 2.1 Hz, 1H), 6.22-6.05 (m, 1H), 4.03 (s, 3H), 2.51 (s, 3H). Example 33B MS: calc'd 416.1 [(M+H)⁺]; measured 416.1 [(M+H)⁺]. ¹H NMR (400 MHz, DMSO-d₆) δ=11.54 (s, 1H), 11.44 (br d, J=6.0 Hz, 1H), 8.70 (s, 1H), 8.37 (d, J=6.1 Hz, 1H), 7.75 (t, J=7.8 Hz, 1H), 7.44 (d, J=7.6 Hz, 1H), 7.29 (d, J=7.6 Hz, 1H), 6.68 (td, J=53.8, 2.6 Hz, 1H), 6.23-6.07 (m, 1H), 4.02 (s, 3H), 2.50 (s, 3H).

### Example 34

### 5-[1-methyl-3-[methyl(1,2,2-trimethylpropyl)amino]pyrazolo[3,4-c]pyridazin-5-yl]-1H-pyrimidine-2,4-dione

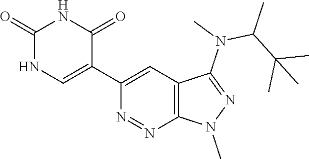 [SMILES: CC(N(C)c1nn(C)c2nnc(-c3c[nH]c(=O)[nH]c3=O)cc12)C(C)(C)C]

The titled compound was synthesized according to the following scheme:

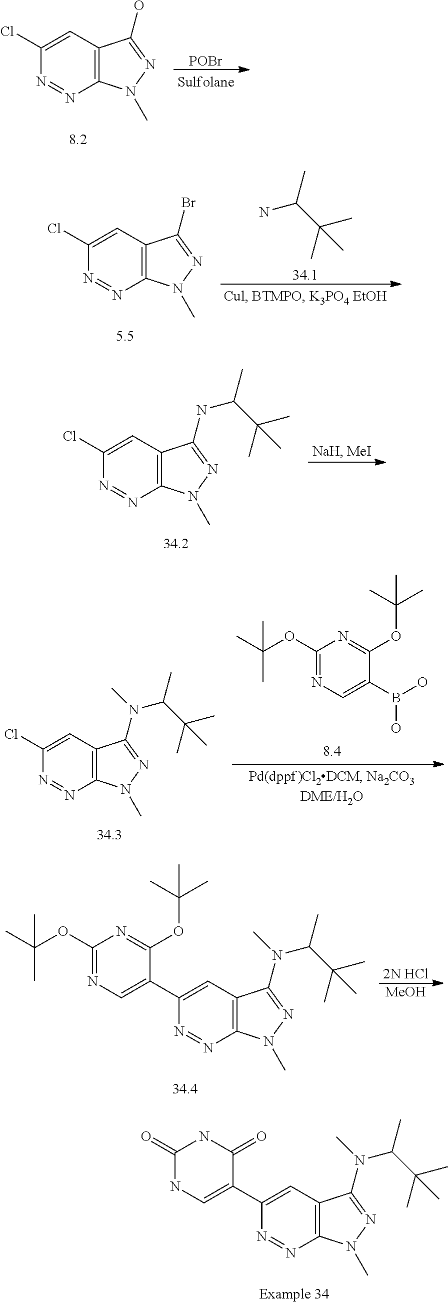

### Step (a): Preparation of 3-bromo-5-chloro-1-methyl-pyrazolo[3,4-c]pyridazine (Compound 5.5)

To a 100 mL round-bottom flask equipped with a magnetic stir bar was added 5-chloro-1-methyl-pyrazolo[3,4-c]pyridazin-3-ol (compound 8.2, 1.0 g, 5.42 mmol) followed by the addition of sulfolane (10 mL). Then POBr₃ (1.71 g, 5.96 mmol) was added into the mixture at 25° C. under nitrogen atmosphere. The mixture was stirred at 50° C. under nitrogen atmosphere for 48 hrs. The mixture was quenched by slow addition of H₂O (100 mL), then extracted with EA (50 mL) for three times. The combined organic layer was washed with the mixture of brine and H₂O (1:1, 50 mL) for five times, dried over anhydrous Na₂SO₄, filtered and concentrated. The residue was purified by silica gel column chromatography (eluent: 1% to 20% EA in PE) to afford compound 5.5 (954.5 mg). MS: calc'd 246.9 [(M+H)⁺]; measured 246.9 [(M+H)⁺].

### Step (b): Preparation of 5-chloro-1-methyl-N-(1,2,2-trimethylpropyl)pyrazolo[3,4-c]pyridazin-3-amine (Compound 34.2)

To a Schlenk flask was added 3-bromo-5-chloro-1-methyl-pyrazolo[3,4-c]pyridazine (compound 5.5, 150 mg, 0.61 mmol), 3,3-dimethylbutan-2-amine (compound 34.1, 92.0 mg, 0.91 mmol), K₃PO₄ (257.3 mg, 1.21 mmol), CuI (5.7 mg, 30.3 μmol), BTMPO (17.8 mg, 42.4 μmol) and EtOH (0.8 mL). The flask was evacuated and backfilled with N₂ for three times, after which the mixture was stirred at 80° C. for 16 hrs. After being cooled to room temperature, the mixture was diluted with EA (50 mL), and filtered through Celite pad and washed with EA. The filtrate was concentrated under vacuum and the residue was purified by silica gel flash column chromatography (eluent: 0% to 50% EA in PE) to afford compound 34.2 (130 mg). MS: calc'd 268.1 [(M+H)⁺]; measured 268.1 [(M+H)⁺].

### Step (c): Preparation of 5-chloro-N,1-dimethyl-N-(1,2,2-trimethylpropyl)pyrazolo[3,4-c]pyridazin-3-amine (Compound 34.3)

To a solution of 5-chloro-1-methyl-N-(1,2,2-trimethylpropyl)pyrazolo[3,4-c]pyridazin-3-amine (compound 34.2, 130 mg, 0.48 mmol) in DMF (3 mL) was added NaH (60% dispersion in mineral oil, 38.8 mg, 0.97 mmol) under 0° C. The mixture was stirred under 0° C. for 15 mins, after which MeI (151 μL, 2.43 mmol) was added. The mixture was warmed up to room temperature slowly and stirred for another 2 hrs. The reaction was quenched by HOAc (30 μL), diluted with 30 mL cold water, and extracted with EA (30 mL) for three times. The combined organic layer was washed with brine, dried over anhydrous Mg₂SO₄ and concentrated. The residue was purified by silica gel flash column chromatography (eluent: 0% to 30% EA in PE) to afford compound 34.3 (120 mg). MS: calc'd 282.1 [(M+H)⁺]; measured 282.1 [(M+H)⁺].

### Step (d): Preparation of 5-(2,4-ditert-butoxypyrimidin-5-yl)-N,1-dimethyl-N-(1,2,2-trimethylpropyl)pyrazolo[3,4-c]pyridazin-3-amine (Compound 34.4)

To a Schlenk flask was added 5-chloro-N,1-dimethyl-N-(1,2,2-trimethylpropyl)pyrazolo[3,4-c]pyridazin-3-amine (compound 34.3, 120 mg, 0.43 mmol), (2,4-ditert-butoxypyrimidin-5-yl)boronic acid (compound 8.4, 173.0 mg, 0.64 mmol), Na₂CO₃ (174.0 mg), Pd(dppf)Cl₂·DCM (31.4 mg, 0.043 mmol). 1,2-Dimethoxyethane (4.0 mL) and water (1.0 ml) were added. The flask was evacuated and backfilled with N₂ for three times, after which the mixture was stirred at 90° C. for 6 hrs. After being cooled to room temperature, the reaction mixture was diluted with water (40 mL), and extracted with EA (50 mL) twice. The combined organic layer was washed with brine, dried over anhydrous Mg₂SO₄, filtered and concentrated. The residue was purified by silica gel flash column chromatography (eluent: 0% to 30% EA in PE) to afford compound 34.4 (177.7 mg).

### Step (e): Preparation of 5-[1-methyl-3-[methyl(1,2,2-trimethylpropyl)amino]pyrazolo[3,4-c]pyridazin-5-yl]-1H-pyrimidine-2,4-dione (Example 34)

A 2N HCl solution in MeOH (1.9 mL) was diluted with 14 mL MeOH. To a solution of 5-(2,4-ditert-butoxypyrimidin-5-yl)-N,1-dimethyl-N-(1,2,2-trimethylpropyl)pyrazolo[3,4-c]pyridazin-3-amine (compound 34.4, 177.7 mg, 378.4 μmol) in MeOH (5 mL) was added the above solution and the mixture was stirred for 3 hr. The desired product precipitated from the red solution. The red solid was filtered and washed with MeOH (2 mL) twice. The solid was dried over oil pump to afford Example 34 (55.4 mg). MS: calc'd 358.4 [(M+H)⁺]; measured 358.1 [(M+H)⁺]. ¹H NMR (400 MHz, DMSO-d6) δ=11.45 (s, 1H), 11.36 (br d, J=5.0 Hz, 1H), 8.85 (s, 1H), 8.30 (d, J=6.3 Hz, 1H), 4.20-4.12 (m, 1H), 3.98 (s, 3H), 3.01 (s, 3H), 1.23-1.20 (m, 3H), 0.98 (s, 9H).

### Example 35

### 5-[3-[1-cyclopentylethyl(methyl)amino]-1-methyl-pyrazolo[3,4-c]pyridazin-5-yl]-1H-pyrimidine-2,4-dione

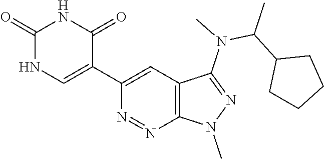 [SMILES: CC(C1CCCC1)N(C)c1nn(C)c2nnc(-c3c[nH]c(=O)[nH]c3=O)cc12]

5-[3-[1-cyclopentylethyl(methyl)amino]-1-methyl-pyrazolo[3,4-c]pyridazin-5-yl]-1H-pyrimidine-2,4-dione (Example 35) was prepared in analogy to Example 34, by replacing 3,3-dimethylbutan-2-amine (compound 34.1) with 1-cyclopentylethanamine in step (b). MS: calc'd 370.4 [(M+H)⁺]; measured 370.1 [(M+H)⁺]. ¹H NMR (400 MHz, DMSO-d₆)=11.45 (br s, 1H), 11.35 (br s, 1H), 8.74 (s, 1H), 8.29 (s, 1H), 3.99 (s, 3H), 3.94-3.81 (m, 1H), 2.92 (s, 3H), 2.21-2.10 (m, 1H), 1.82-1.73 (m, 1H), 1.66 (ddd, J=12.2, 8.0, 4.4 Hz, 2H), 1.60-1.45 (m, 3H), 1.29-1.20 (m, 2H), 1.16 (d, J=6.5 Hz, 3H).

### Example 36

### 5-[3-[cyclopentyl(methyl)amino]-1-methyl-pyrazolo[3,4-c]pyridazin-5-yl]-1H-pyrimidine-2,4-dione

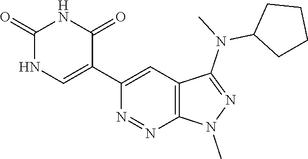 [SMILES: CN(c1nn(C)c2nnc(-c3c[nH]c(=O)[nH]c3=O)cc12)C1CCCC1]

5-[3-[cyclopentyl(methyl)amino]-1-methyl-pyrazolo[3,4-c]pyridazin-5-yl]-1H-pyrimidine-2,4-dione (Example 36) was prepared in analogy to Example 34, by replacing 3,3-dimethylbutan-2-amine (compound 34.1) with cyclopentanamine in step (b). MS: calc'd 342.3 [(M+H)⁺]; measured 342.1 [(M+H)⁺]. ¹H NMR (400 MHz, DMSO-d₆) δ=11.45 (s, 1H), 11.36 (br d, J=5.25 Hz, 1H), 8.69 (s, 1H), 8.26 (d, J=6.1 Hz, 1H), 4.49 (br t, J=7.6 Hz, 1H), 4.00 (s, 3H), 2.96 (s, 3H), 1.91-1.80 (m, 2H), 1.75-1.56 (m, 6H).

### Example 37

### 5-[1-methyl-3-[[(1S)-1-phenylethyl]amino]pyrazolo[3,4-c]pyridazin-5-yl]-1H-pyrimidine-2,4-dione

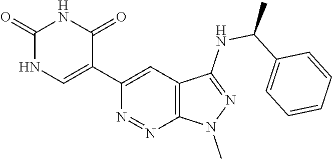 [SMILES: C[C@H](Nc1nn(C)c2nnc(-c3c[nH]c(=O)[nH]c3=O)cc12)c1ccccc1]

The titled compound was synthesized according to the following scheme:

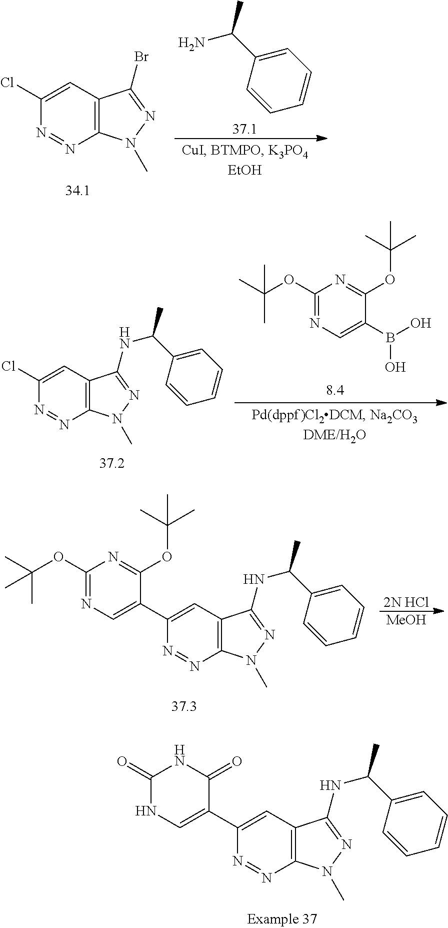

### Step (a): Preparation of 5-chloro-1-methyl-N-[(1S)-1-phenylethyl]pyrazolo[3,4-c]pyridazin-3-amine (Compound 37.2)

To a Schlenk flask was added 3-bromo-5-chloro-1-methyl-pyrazolo[3,4-c]pyridazine (compound 5.5, 500 mg, 2.0 mmol), (1S)-1-phenylethanamine (compound 37.1, 367.2 mg, 3.0 mmol), K₃PO₄ (857.7 mg, 4.0 mmol), CuI (19.2 mg, 101.0 μmol), BTMPO (42.5 mg, 101.0 μmol) and EtOH (3.0 mL). The flask was evacuated and backfilled with N₂ for three times, after which the mixture was stirred at 85° C. for 16 hrs. After being cooled to room temperature, the reaction mixture was diluted with water (40 mL), and extracted with EA (50 mL) twice. The combined organic layer was washed with brine, dried over anhydrous Mg₂SO₄, filtered and concentrated. The residue was purified by silica gel flash column chromatography (eluent: 0% to 30% EA in PE) to afford compound 37.2 (400 mg). MS: calc'd 288.1 [(M+H)⁺]; measured 288.1 [(M+H)⁺].

### Step (b): Preparation of 5-(2,4-ditert-butoxypyrimidin-5-yl)-1-methyl-N-[(1S)-1-phenylethyl]pyrazolo[3,4-c]pyridazin-3-amine (Compound 37.3)

To a Schlenk flask was added 5-chloro-1-methyl-N-[(1S)-1-phenylethyl]pyrazolo[3,4-c]pyridazin-3-amine (compound 37.2, 50.0 mg, 173.7 μmol), (2,4-ditert-butoxypyrimidin-5-yl)boronic acid (compound 8.4, 69.9 mg, 260.6 μmol), Na₂CO₃ (73.7 mg, 695.1 μmol), Pd(dppf)Cl₂·DCM (12.7 mg, 17.4 μmol). 1,2-dimethoxyethane (1.0 mL) and water (0.25 ml) were added. The flask was evacuated and backfilled with N₂ for three times, after which the mixture was stirred at 90° C. for 6 hrs. After being cooled to room temperature, the reaction mixture was diluted with water (40 mL), and extracted with EA (50 mL) twice. The combined organic layer was washed with brine, dried over anhydrous Mg₂SO₄, filtered and concentrated.

The residue was purified by silica gel flash column chromatography (eluent: 0% to 30% EA in PE) to afford compound 37.3 (67.0 mg).

### Step (c): Preparation of 5-[1-methyl-3-[[(1S)-1-phenylethyl]amino]pyrazolo[3,4-c]pyridazin-5-yl]-1H-pyrimidine-2,4-dione (Example 37)

A 2N HCl solution in MeOH (0.7 mL) was diluted with 2.5 mL MeOH. To a solution of 5-(2,4-ditert-butoxypyrimidin-5-yl)-1-methyl-N-[(1S)-1-phenylethyl]pyrazolo[3,4-c]pyridazin-3-amine (compound 37.3, 67.0 mg, 140.9 μmol) in MeOH (1 mL) was added the above solution and the mixture was stirred for 40 min. The desired product precipitated from the red solution. The red solid was filtered and washed with MeOH (1 mL) twice, then dried over oil pump to afford Example 37 (30.0 mg). MS: calc'd 364.2 [(M+H)⁺]; measured 364.1 [(M+H)⁺]. ¹H NMR (400 MHz, DMSO-d₆) δ=11.45 (s, 1H), 11.33 (br d, J=4.9 Hz, 1H), 8.81 (s, 1H), 8.22 (d, J=6.1 Hz, 1H), 7.46-7.39 (m, 3H), 7.30 (t, J=7.6 Hz, 2H), 7.23-7.17 (m, 1H), 4.90 (quin, J=6.7 Hz, 1H), 3.87 (s, 3H), 1.50 (d, J=6.9 Hz, 3H).

### Example 38

### 5-[1-methyl-3-[methyl-[(1S)-1-phenylethyl]amino]pyrazolo[3,4-c]pyridazin-5-yl]-1H-pyrimidine-2,4-dione

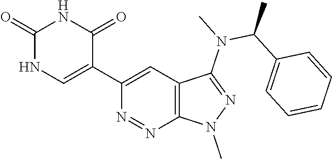 [SMILES: C[C@@H](c1ccccc1)N(C)c1nn(C)c2nnc(-c3c[nH]c(=O)[nH]c3=O)cc12]

5-[1-methyl-3-[methyl-[(1S)-1-phenylethyl]amino]pyrazolo[3,4-c]pyridazin-5-yl]-1H-pyrimidine-2,4-dione (Example 38) was prepared in analogy to Example 34, by replacing 3,3-dimethylbutan-2-amine (compound 34.1) with (1S)-1-phenylethanamine in step (b). MS: calc'd 378.2 [(M+H)⁺]; measured 378.2 [(M+H)⁺]. ¹H NMR (500 MHz, DMSO-d₆) δ=11.46 (s, 1H), 11.37 (br d, J=4.6 Hz, 1H), 8.73 (s, 1H), 8.25 (d, J=6.1 Hz, 1H), 7.39-7.33 (m, 4H), 7.31-7.24 (m, 1H), 5.47 (q, J=6.9 Hz, 1H), 4.02 (s, 3H), 2.85 (s, 3H), 1.59 (d, J=7.0 Hz, 3H).

### Example 39

### 5-(3-cyclobutyl-1-methyl-pyrazolo[3,4-c]pyridazin-5-yl)-1H-pyrimidine-2,4-dione

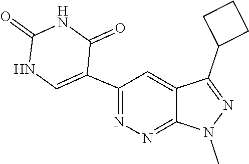 [SMILES: Cn1nc(C2CCC2)c2cc(-c3c[nH]c(=O)[nH]c3=O)nnc21]

The titled compound was synthesized according to the following scheme:

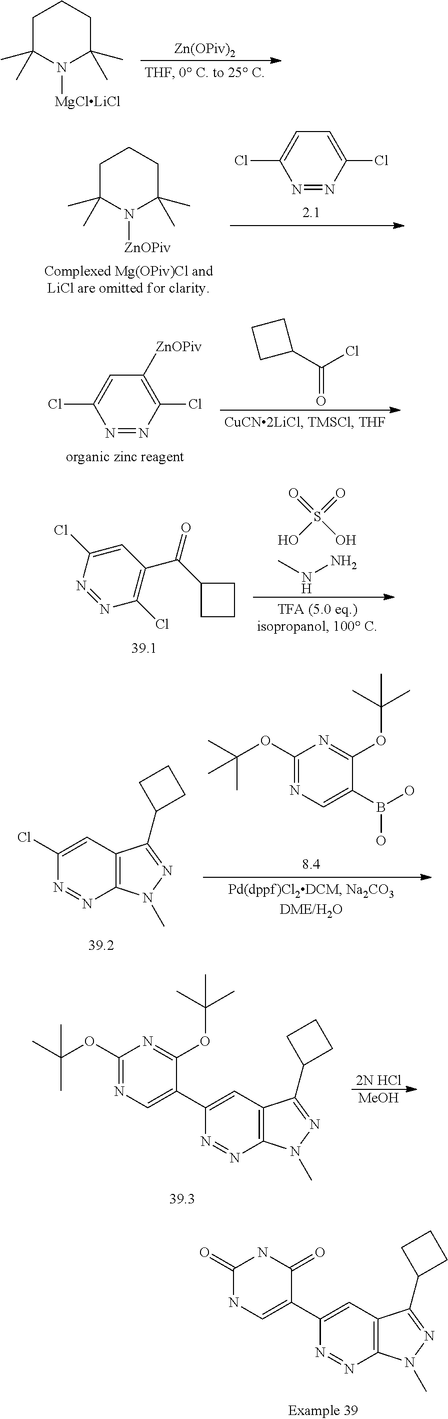

### Step (a): Preparation of cyclobutyl-(3,6-dichloropyridazin-4-yl)methanone (Compound 39.1)

A dry and N₂ flushed flask was charged with a solution of TMPMgCl·LiCl (1 M in THF, 7.7 mL, 7.7 mmol) and was cooled to 0° C. using ice bath. To the mixture was added pre-treated Zn(OPiv)₂ in one portion (zinc pivalate, 2.2 g, 8.05 mmol, dried in vacuo at 400° C. or with a heat gun at 300° C. for 30 min prior to use), and the mixture was warmed up to 25° C. slowly over 1.5 hr to afford a brown solution as freshly prepared TMPZnOPiv·Mg(OPiv)Cl·LiCl at a cal. 1 M concentration.

3,6-dichloropyridazine (compound 2.1, 1.04 g, 7.0 mmol) was dissolved in 14 mL dry THF. To this solution was added the freshly prepared TMPZnOPiv·Mg(OPiv)Cl·LiCl solution dropwise at 25° C. and was stirred for 0.5 h to get the desired organic zinc reagent.

The above zinc reagent was cooled to at −20° C., and TMSCl (3.6 mL, 42 mmol) was added in one portion and kept stirring for 0.5 h. Then CuCN·2LiCl (1M in THF, 7.0 mL) and cyclobutanecarbonyl chloride (18.2 mmol) was added subsequently. The resultant mixture was stirred at −20° C. for 1 h and at 0° C. for 15 h. Then the mixture was quenched with an aqueous solution of sat. NH₄Cl/NH₃ (conc.) (v/v, 8:1; 56 mL) and extracted with ethyl acetate, washed with aqueous solution of KHCO₃ (to remove the unreacted alkyl acid), brine and dried over Na₂SO₄ to get the crude product. The crude product was purified with silica gel flash chromatography (eluent, 10% to 25% EA in PE) to afford Compound 39.1 (700 mg). MS: calc'd 231.0 [(M+H)⁺]; measured 231.0 [(M+H)⁺].

### Step (b): Preparation of 5-chloro-3-cyclobutyl-1-methyl-pyrazolo[3,4-c]pyridazine (Compound 39.2)

Cyclobutyl-(3,6-dichloropyridazin-4-yl)methanone (compound 39.1, 70 mg, 303.0 μmol) and methylhydrazine sulfate (48.0 mg, 333.2 μmol) were added to a flask with a stirring bar. To the mixture was added isopropanol (1.5 mL) and TFA (117 μL). The mixture was stirred at 100° C. for 6 hr. After concentration, the crude residue was purified with silica gel flash chromatography (eluent, 10% to 25% EA in PE) to afford compound 39.2 (48 mg). MS: calc'd 223.1 [(M+H)⁺]; measured 223.1 [(M+H)⁺].

### Step (c): Preparation of 3-cyclobutyl-5-(2,4-ditert-butoxypyrimidin-5-yl)-1-methyl-pyrazolo[3,4-c]pyridazine (Compound 39.3)

To a Schlenk flask was added 5-chloro-3-cyclobutyl-1-methyl-pyrazolo[3,4-c]pyridazine (compound 39.2, 48.0 mg, 215.6 μmol), (2,4-ditert-butoxypyrimidin-5-yl)boronic acid (compound 8.4, 86.7 mg, 323.3 μmol), Na₂CO₃ (91.4 mg, 862.2 μmol), Pd(dppf)Cl₂·DCM (15.7 mg, 21.5 μmol). 1,2-dimethoxyethane (4.0 mL) and water (1.0 mL) were added. The flask was evacuated and backfilled with N₂ for three times, after which the mixture was stirred at 90° C. for 6 hrs. After being cooled to room temperature, the reaction mixture was diluted with water (40 mL), and extracted with EA (50 mL) twice. The combined organic layer was washed with brine, dried over anhydrous Mg₂SO₄, filtered and concentrated. The residue was purified by silica gel flash column chromatography (eluent: 0% to 25% EA in PE) to afford compound 39.3 (70.0 mg).

### Step (d): Preparation of 5-(3-cyclobutyl-1-methyl-pyrazolo[3,4-c]pyridazin-5-yl)-1H-pyrimidine-2,4-dione (Example 39)

A 2N HCl solution in MeOH (0.9 mL) was diluted with 2.0 mL MeOH. To a solution of 3-cyclobutyl-5-(2,4-ditert-butoxypyrimidin-5-yl)-1-methyl-pyrazolo[3,4-c]pyridazine (compound 39.3, 70.0 mg, 162.0 μmol) in MeOH (2.5 mL) was added the above solution and the mixture was stirred for 40 min until the completion of the reaction. The desired product was precipitated from the solution. The solid was filtered and washed with MeOH (1 mL) twice. The solid was dried over oil pump to afford Example 39 (40.0 mg). MS: calc'd 299.1 [(M+H)⁺]; measured 299.2 [(M+H)⁺]. ¹H NMR (400 MHz, DMSO-d₆) δ=11.49 (s, 1H), 11.40 (br d, J=5.3 Hz, 1H), 8.68 (s, 1H), 8.32 (d, J=6.1 Hz, 1H), 4.20 (s, 3H), 4.00-3.91 (m, 1H), 2.48-2.35 (m, 4H), 2.17-2.06 (m, 1H), 2.01-1.92 (m, 1H).

### Example 40

### 5-[3-[(1S)-1-(5-fluoro-6-methyl-2-pyridyl)ethoxy]-1-methyl-pyrazolo[3,4-c]pyridazin-5-yl]-1H-pyrimidine-2,4-dione

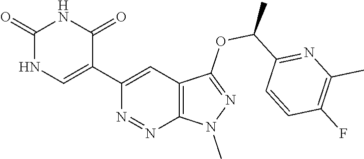 [SMILES: Cc1nc([C@H](C)Oc2nn(C)c3nnc(-c4c[nH]c(=O)[nH]c4=O)cc23)ccc1F]

The titled compound was synthesized according to the following scheme:

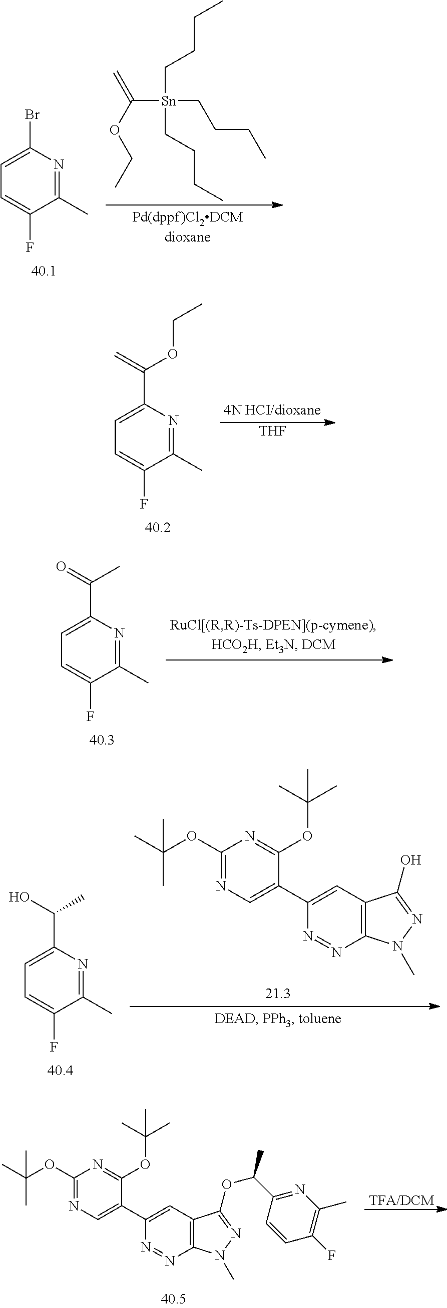

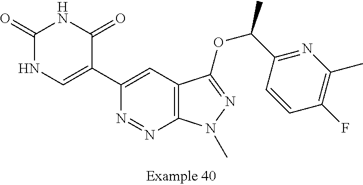 [SMILES: Cc1nc([C@H](C)Oc2nn(C)c3nnc(-c4c[nH]c(=O)[nH]c4=O)cc23)ccc1F]

### Step (a): Preparation of 6-(1-ethoxyvinyl)-3-fluoro-2-methyl-pyridine (Compound 40.2)

The mixture of Pd(dppf)Cl₂·DCM (214.89 mg, 263.14 μmol), 1-ethoxyvinyltri-n-butyltin (950.34 mg, 896.55 μL, 2.63 mmol) and 6-bromo-3-fluoro-2-methyl-pyridine (compound 40.1, 500 mg, 2.63 mmol) in 1,4-dioxane (10 mL) was stirred at 100° C. for 20 hr. After being cooled to room temperature, the reaction mixture was diluted with EA (50 mL), and treated with a 10% aqueous KF (50 mL). The resultant mixture was stirred at room temperature for 1 h, the mixture was filtered, the solid was washed with EA. The resultant organic layer was concentrated, the crude residue was purified by flash chromatography (silica gel, 24 g, 0 to 100% EA in PE) to afford compound (420 mg). MS: calc'd 182.1 [(M+H)⁺]; measured 182.2 [(M+H)⁺].

### Step (b): Preparation of 1-(5-fluoro-6-methyl-2-pyridyl)ethanone (Compound 40.3)

To a solution of 6-(1-ethoxyvinyl)-3-fluoro-2-methyl-pyridine (compound 40.2, 370 mg, 2.04 mmol) in THF (20 mL) was added 4N HCl/dioxane (2 mL), the resultant mixture was stirred at room temperature for 1 h. The reaction mixture was quenched with aq. NaHCO₃ (50 mL), extracted with EA (80 mL) twice. The combined organic layer was washed with brine, dried over Na₂SO₄, filtered and concentrated. The residue was purified by flash chromatography (silica gel, 25 g, 0 to 100% EA in PE) to afford compound 40.3 (260 mg). MS: calc'd 154.1 [(M+H)⁺]; measured 154.2 [(M+H)⁺].

### Step (c): Preparation of (1R)-1-(5-fluoro-6-methyl-2-pyridyl)ethanol (Compound 40.4)

To a solution of 1-(5-fluoro-6-methyl-2-pyridyl)ethanone (compound 40.3, 260 mg, 1.7 mmol) in DCM (10 mL) was added Et₃N (687.15 mg, 946.49 μL, 6.79 mmol), formic acid (781.44 mg, 651.2 μL, 16.98 mmol) and RuCl[(R,R)-TsDPEN(p-cymene)](10.8 mg, 16.98 μmol) at 0° C., the resultant mixture was stirred at room temperature overnight. The reaction mixture was concentrated. The residue was purified by flash chromatography (silica gel, 12 g, 0 to 20% MeOH in DCM) to afford compound 40.4 (110 mg).

### Step (d): Preparation of 5-(2,4-ditert-butoxypyrimidin-5-yl)-1-methyl-3-[(1S)-1-(5-fluoro-6-methyl-2-pyridyl)ethoxy]pyrazolo[3,4-c]pyridazine (Compound 40.5)

To a suspension of (1R)-1-(5-fluoro-6-methyl-2-pyridyl)ethanol (compound 40.4, 68.8 mg, 443.1 μmol), 5-(2,4-ditert-butoxypyrimidin-5-yl)-1-methyl-pyrazolo[3,4-c]pyridazin-3-ol (compound 21.3, 110 mg, 295.4 μmol) and Ph₃P (131.7 mg, 502.1 μmol) in toluene (10 mL) was added DEAD (90.8 mg, 82.5 μL, 521.1 μmol), the resultant mixture was stirred at 60° C. for 3 h under N₂ atmosphere. After being cooled to room temperature, the reaction mixture was diluted with water (30 mL), and extracted with EA (30 mL) for three times. The combined organic layer was concentrated, and the residue was purified by flash chromatography (silica gel, 25 g, 0 to 100% EA in PE) to afford compound 40.5 (70 mg). MS: calc'd 510.2 [(M+H)⁺]; measured 510.3 [(M+H)⁺].

### Step (e): Preparation of 5-[3-[(1S)-1-(5-fluoro-6-methyl-2-pyridyl)ethoxy]-1-methyl-pyrazolo[3,4-c]pyridazin-5-yl]-1H-pyrimidine-2,4-dione (Compound 40.5) 398.1

To a solution of 5-(2,4-ditert-butoxypyrimidin-5-yl)-1-methyl-3-[(1S)-1-(5-fluoro-6-methyl-2-pyridyl)ethoxy]pyrazolo[3,4-c]pyridazine (compound 40.5, 70 mg, 137.4 μmol) in DCM (5 mL) was added TFA (0.5 mL). The reaction mixture was stirred at room temperature for 1 h, then concentrated to afford a crude product, which was purified by pre-HPLC to afford Example 40 (45 mg). MS: calc'd 398.1 [(M+H)⁺]; measured 398.2 [(M+H)⁺]. ¹H NMR (400 MHz, DMSO-d₆) δ=11.51 (s, 1H), 11.42 (br d, J=5.6 Hz, 1H), 8.62 (s, 1H), 8.34 (d, J=6.3 Hz, 1H), 7.61 (t, J=9.1 Hz, 1H), 7.50-7.37 (m, 1H), 6.08-5.88 (m, 1H), 4.01 (s, 3H), 2.46 (d, J=3.0 Hz, 3H), 1.71 (d, J=6.5 Hz, 3H).

### Example 41

### 5-[1-methyl-3-[(1S)-1-(6-methyl-2-pyridyl)ethoxy]pyrazolo[3,4-c]pyridazin-5-yl]-1H-pyrimidine-2,4-dione

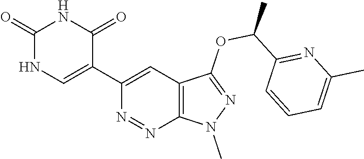 [SMILES: Cc1cccc([C@H](C)Oc2nn(C)c3nnc(-c4c[nH]c(=O)[nH]c4=O)cc23)n1]

5-[1-methyl-3-[(1S)-1-(6-methyl-2-pyridyl)ethoxy]pyrazolo[3,4-c]pyridazin-5-yl]-1H-pyrimidine-2,4-dione (Example 41) was prepared in analogy to Example 40, by replacing 1-(5-fluoro-6-methyl-2-pyridyl)ethanone (compound 40.3) with 1-(6-methyl-2-pyridyl)ethanone in step (c). MS: calc'd 380.1 [(M+H)⁺]; measured 380.1 [(M+H)⁺]. ¹H NMR (400 MHz, DMSO-d₆) δ=11.52 (s, 1H), 11.47 (br d, J=5.8 Hz, 1H), 8.67 (s, 1H), 8.36 (d, J=6.1 Hz, 1H), 8.05 (br s, 1H), 7.67 (br d, J=6.5 Hz, 1H), 7.51 (br d, J=6.9 Hz, 1H), 6.18-6.08 (m, 1H), 4.01 (s, 3H), 2.64 (s, 3H), 1.78 (d, J=6.5 Hz, 3H).

### Example 42

### 5-[1-methyl-3-[(1S)-1-(4-methyl-2-pyridyl)ethoxy]pyrazolo[3,4-c]pyridazin-5-yl]-1H-pyrimidine-2,4-dione

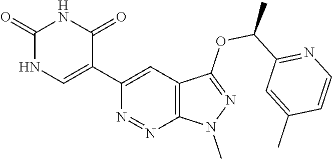 [SMILES: Cc1ccnc([C@H](C)Oc2nn(C)c3nnc(-c4c[nH]c(=O)[nH]c4=O)cc23)c1]

5-[1-methyl-3-[(1S)-1-(4-methyl-2-pyridyl)ethoxy]pyrazolo[3,4-c]pyridazin-5-yl]-1H-pyrimidine-2,4-dione (Example 42) was prepared in analogy to Example 40, by replacing 1-(5-fluoro-6-methyl-2-pyridyl)ethanone (compound 40.3) with 1-(4-methyl-2-pyridyl)ethanone in step (c). MS: calc'd 380.1 [(M+H)⁺]; measured 380.1 [(M+H)⁺]. ¹H NMR (400 MHz, DMSO-d₆) δ=11.53 (s, 1H), 11.47 (br d, J=5.9 Hz, 1H), 8.68 (s, 1H), 8.59 (d, J=5.5 Hz, 1H), 8.36 (d, J=6.3 Hz, 1H), 7.77 (br s, 1H), 7.52 (br d, J=3.5 Hz, 1H), 6.18-6.06 (m, 1H), 4.01 (s, 3H), 2.46 (s, 3H), 1.77 (d, J=6.6 Hz, 3H).

### Example 43

### 5-[3-[(1S)-1-(5-fluoro-2-pyridyl)ethoxy]-1-methyl-pyrazolo[3,4-c]pyridazin-5-yl]-1H-pyrimidine-2,4-dione

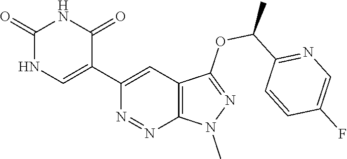 [SMILES: C[C@H](Oc1nn(C)c2nnc(-c3c[nH]c(=O)[nH]c3=O)cc12)c1ccc(F)cn1]

5-[3-[(1S)-1-(5-fluoro-2-pyridyl)ethoxy]-1-methyl-pyrazolo[3,4-c]pyridazin-5-yl]-1H-pyrimidine-2,4-dione (Example 43) was prepared in analogy to Example 40, by replacing 1-(5-fluoro-6-methyl-2-pyridyl)ethanone (compound 40.3) with 1-(5-fluoro-2-pyridyl)ethanone in step (c). MS: calc'd 384.1 [(M+H)⁺]; measured 384.1[(M+H)⁺]. ¹H NMR (400 MHz, DMSO-d₆) δ=11.52 (s, 1H), 11.44 (br d, J=6.0 Hz, 1H), 8.63 (s, 1H), 8.57 (d, J=2.9 Hz, 1H), 8.34 (d, J=6.3 Hz, 1H), 7.76-7.70 (m, 1H), 7.68-7.62 (m, 1H), 6.08-6.00 (m, 1H), 4.01 (s, 3H), 1.72 (d, J=6.5 Hz, 3H).

### Example 44

### 5-[3-[(1S)-1-(6-chloro-2-pyridyl)ethoxy]-1-methyl-pyrazolo[3,4-c]pyridazin-5-yl]-1H-pyrimidine-2,4-dione

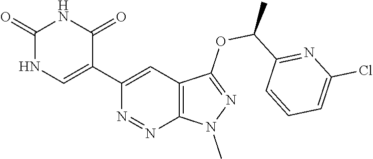 [SMILES: C[C@H](Oc1nn(C)c2nnc(-c3c[nH]c(=O)[nH]c3=O)cc12)c1cccc(Cl)n1]

5-[3-[(1S)-1-(6-chloro-2-pyridyl)ethoxy]-1-methyl-pyrazolo[3,4-c]pyridazin-5-yl]-1H-pyrimidine-2,4-dione (Example 44) was prepared in analogy to Example 40, by replacing 1-(5-fluoro-6-methyl-2-pyridyl)ethanone (compound 40.3) with 1-(6-chloro-2-pyridyl)ethanone in step (c). MS: calc'd 400.1, 402.1 [(M+H)⁺]; measured 400.1, 402.1 [(M+H)⁺]. ¹H NMR (400 MHz, DMSO-d₆) δ=11.52 (s, 1H), 11.43 (br d, J=5.4 Hz, 1H), 8.65 (s, 1H), 8.35 (d, J=6.3 Hz, 1H), 7.87 (t, J=7.8 Hz, 1H), 7.57 (d, J=7.5 Hz, 1H), 7.46 (d, J=7.9 Hz, 1H), 6.01-5.92 (m, 1H), 4.01 (s, 3H), 1.72 (d, J=6.5 Hz, 3H)

### Example 45

### 5-[3-[(1S)-1-(6-chloropyridazin-3-yl)ethoxy]-1-methyl-pyrazolo[3,4-c]pyridazin-5-yl]-1H-pyrimidine-2,4-dione

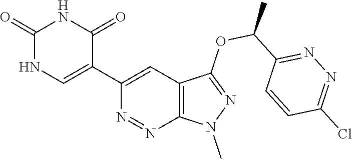 [SMILES: C[C@H](Oc1nn(C)c2nnc(-c3c[nH]c(=O)[nH]c3=O)cc12)c1ccc(Cl)nn1]

5-[3-[(1S)-1-(6-chloropyridazin-3-yl)ethoxy]-1-methyl-pyrazolo[3,4-c]pyridazin-5-yl]-1H-pyrimidine-2,4-dione (Example 45) was prepared in analogy to Example 40, by replacing 1-(5-fluoro-6-methyl-2-pyridyl)ethanone (compound 40.3) with 1-(6-chloropyridazin-3-yl)ethanone in step (c). MS: calc'd 401.1, 403.1 [(M+H)⁺]; measured 401.0, 403.0 [(M+H)⁺]. ¹H NMR (500 MHz, DMSO-d₆) δ=11.52 (s, 1H), 11.44 (br d, J=6.0 Hz, 1H), 8.66 (s, 1H), 8.35 (d, J=6.3 Hz, 1H), 8.03-7.98 (m, 1H), 7.95-7.92 (m, 1H), 6.26-6.20 (m, 1H), 4.00 (s, 3H), 1.81 (d, J=6.6 Hz, 3H).

### Example 46

### 5-(3-cyclobutylisoxazolo[5,4-c]pyridazin-5-yl)-1H-pyrimidine-2,4-dione

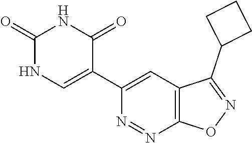 [SMILES: O=c1[nH]cc(-c2cc3c(C4CCC4)noc3nn2)c(=O)[nH]1]

The titled compound was synthesized according to the following scheme:

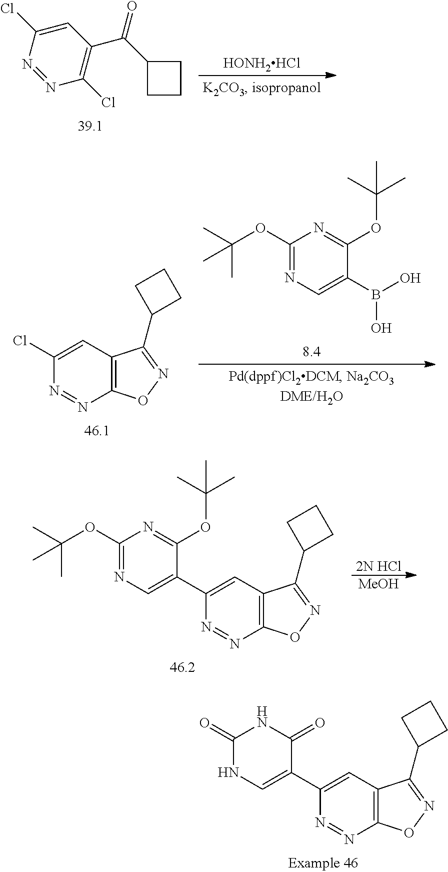

### Step (a): Preparation of 5-chloro-3-cyclobutyl-isoxazolo[5,4-c]pyridazine (Compound 46.1)

Cyclobutyl-(3,6-dichloropyridazin-4-yl)methanone (compound 39.1, 50 mg, 303.0 μmol), hydroxylamine hydrochloride (16.5 mg, 238.1 μmol) and K₂CO₃ (59.8 mg, 432.7 μmol) were added to a flask with a stirring bar. To the mixture was added isopropanol (1.0 mL). The mixture was stirred at 100° C. for 2 hr. After concentration, the crude residue was purified with silica gel flash chromatography (eluent, 0% to 10% EA in PE) to get compound 46.1 (30 mg). MS: calc'd 210.0 [(M+H)⁺]; measured 210.0 [(M+H)⁺].

### Step (b): Preparation 3-cyclobutyl-5-(2,4-ditert-butoxypyrimidin-5-yl)isoxazolo[5,4-c]pyridazine (Compound 46.2)

To a Schlenk flask was added 5-chloro-3-cyclobutyl-isoxazolo[5,4-c]pyridazine (compound 46.1, 30.0 mg, 143.1 μmol), (2,4-ditert-butoxypyrimidin-5-yl)boronic acid (compound 8.4, 50.0 mg, 186.4 μmol), Na₂CO₃ (60.7 mg, 572.4 μmol), Pd(dppf)Cl₂·DCM (10.5 mg, 14.3 μmol). 1,2-Dimethoxyethane (2.0 mL) and water (0.5 mL) were added. The flask was evacuated and backfilled with N₂ for three times, after which the mixture was stirred at 90° C. for 6 hrs. After being cooled to room temperature, the reaction mixture was diluted with water (40 mL), and extracted with EA (50 mL) twice. The combined organic layer was washed with brine, dried over anhydrous Mg₂SO₄, filtered and concentrated. The residue was purified by silica gel flash column chromatography (eluent: 0% to 10% EA in PE) to afford compound 46.2 (23.0 mg).

### Step (c): Preparation of 5-(3-cyclobutylisoxazolo[5,4-c]pyridazin-5-yl)-1H-pyrimidine-2,4-dione (Example 46)

A 2N HCl solution in MeOH (0.3 mL) was diluted with 1.0 mL MeOH. To a solution of 3-cyclobutyl-5-(2,4-ditert-butoxypyrimidin-5-yl)isoxazolo[5,4-c]pyridazine (compound 46.2, 23.0 mg, 57.7 μmol) in MeOH (1.0 mL) was added the above solution and the mixture was stirred for 40 mins until the completion of the reaction. After concentration, the desired product was purified via silica gel flash column chromatography (0% to 10% MeOH in CH₂Cl₂) to afford Example 46 (5.0 mg). MS: calc'd 286.1 [(M+H)⁺]; measured 286.0 [(M+H)⁺]. ¹H NMR (400 MHz, DMSO-d₆) δ=11.58 (br s, 2H), 8.83 (s, 1H), 8.38 (s, 1H), 4.06 (dt, J=0.8, 8.5 Hz, 1H), 2.50-2.40 (m, 4H), 2.22-2.14 (m, 1H), 2.06-2.02 (m, 1H).

### Biological Example

### Example 47: Human Microsomal Stability Assay

Human liver microsomes (Cat. NO.: 452117, Corning, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold ACN (including internal standard) was added to 100 μL incubation mixture to terminate the reaction. Following precipitation and centrifugation, 100 μL supernatant will be taken out and added 300 μL water. The amount of compound remaining in the samples was determined by LC-MS/MS. Controls of no NADPH regenerating system at zero and 30 minutes were also prepared and analyzed. The results were categorized as: low (<7.0 mL/min/kg), medium (7.0-16.2 mL/min/kg) and high (16.2-23.2 mL/min/kg). Test results were summarized in Table 1.

TABLE 1
Human microsomal stability results

| Example No | CL (h) (mL/min/kg) |
|---|---|
| 1 | 8.14 |
| 2 | 8.52 |
| 4 | 6.15 |
| 5 | 6.15 |
| 6 | 6.15 |
| 7 | 9.85 |
| 9 | 6.15 |
| 10 | 6.15 |
| 11 | 6.15 |
| 12 | 6.15 |
| 13 | 7.84 |
| 14 | 7.01 |
| 15 | 6.15 |
| 16 | 6.15 |
| 17 | 6.15 |
| 18 | 6.22 |
| 19 | 9.03 |
| 20 | 6.36 |
| 21 | 6.32 |
| 22 | 8.14 |
| 24 | 6.26 |
| 25 | 8.85 |
| 26 | 6.15 |
| 27 | 6.15 |
| 28 | 6.15 |
| 29A | 6.15 |
| 29B | 6.15 |
| 30A | 6.15 |
| 30B | 8.44 |
| 31A | 6.15 |
| 31B | 6.15 |
| 32A | 6.15 |
| 32B | 6.15 |
| 33A | 6.15 |
| 33B | 6.15 |
| 36 | 6.15 |
| 37 | 6.15 |
| 38 | 9.3 |
| 39 | 6.15 |
| 40 | 6.15 |
| 41 | 8.11 |
| 42 | 7.78 |
| 43 | 6.15 |
| 44 | 6.15 |
| 45 | 6.15 |

### Example 48: CD73 Cellular Assay

Compound serial dilution (1:3) was prepared with Echo 555 liquid handler (Labcyte) into the corresponding wells of a 384-well plate. 40 μL of MDA-MB-231 cells (ATCC, HTB-26, breast cancer, final concentration at 20,000 cells/mL) suspended in assay buffer (20 mM HEPES pH 7.4, 137 mM NaCl, 5.4 mM KCl, 1.3 mM CaCl2, 4.2 mM NaHCO3, 1 mg/mL glucose) were added into the corresponding wells of the plate. After incubating with compounds for 30 minutes, 40 μL of AMP working solution (200 μM AMP in assay buffer) was added into each well in the assay plate. The assay plate was then incubated in a 5% CO₂ incubator (Thermo Fisher Scientific) at 37° C. for 45 min. After the reaction was completed, 50 μL of the supernatants was collected and transferred into a new 384 well plate. 10 μL of Malachite A was added to each well in the assay plate and incubated for 10 minutes. 10 μL of Malachite B was then added to each corresponding well of the plate and incubated for 30 minutes. Finally the absorbance value was read on the Envision plate reader at 620 nM. Calculation percentage inhibition by using equation {% inhibition=100×[I−(X−MIN)/(MAX−MIN)]} where X equals to the well signal, Max equals signal of neutral control and MIN equals signal of inhibitor control.

TABLE 2
Cellular IC₅₀ values of the compounds
of this invention against CD73

| Example No | IC₅₀ (nM) |
|---|---|
| 1 | 56.9 |
| 2 | 10.2 |
| 3 | 103.7 |
| 4 | 158.6 |
| 5 | 7.7 |
| 6 | 12.9 |
| 7 | 11.4 |
| 8 | 13.3 |
| 9 | 46.7 |
| 10 | 2.8 |
| 11 | 2.9 |
| 12 | 3.4 |
| 13 | 5.5 |
| 14 | 4.0 |
| 15 | 4.1 |
| 16 | 3.5 |
| 17 | 3.7 |
| 18 | 4.3 |
| 19 | 3.9 |
| 20 | 6.9 |
| 21 | 4.6 |
| 22 | 3.9 |
| 23 | 58.6 |
| 24 | 19.6 |
| 25 | 8.0 |
| 26 | 2.7 |
| 27 | 1.6 |
| 28 | 0.6 |
| 29A | 3.4 |
| 30A | 1.6 |
| 31A | 1.0 |
| 32A | 0.7 |
| 33A | 0.8 |
| 34 | 5.5 |
| 35 | 2.1 |
| 36 | 8.6 |
| 37 | 4.0 |
| 38 | 1.6 |
| 39 | 3.5 |
| 40 | 1.3 |
| 41 | 1.4 |
| 42 | 4.1 |
| 43 | 3.7 |
| 44 | 2.3 |
| 45 | 9.9 |
| 46 | 10.4 |

### Example 49: CD73 Potency Assay Using LC/MS

The purpose of this assay is to identify and characterize inhibitors of CD73 enzymatic activity. Compound serial dilution (1:3) was prepared with Echo 555 liquid handler (Labcyte) into the corresponding wells of a 384-well plate. 10 μL of enzyme working solution (containing 0.05 nM recombinant CD73 protein, 10 mM Tris pH 7.5, 100 mM NaCl, 0.01% BSA, 0.2 mM Octyl glucoside) was added to the assay plate and incubated with compounds for 15 minutes at room temperature. After adding 15 μL of AMP working solution (Containing 10 mM Tris pH 7.5, 100 mM NaCl, 0.01% BSA, 0.2 mM Octyl glucoside, with 1000 μM of AMP final concentration) and incubate for 10 min at room temperature. Stop the reaction by adding 75 μL of stop solution (5% TCA in H₂O containing 250 nM 13C5-adenosine) to each well for 10 min incubation. After centrifugation, 75 μL of the mixture was transferred to a new 384-well plate for LC/MS analysis.

Samples from the 384-well plates, were loaded onto an autosampler deck, then injected with ADDA-LC-MS/MS. The aqueous mobile phase is 0.1% formic acid in water. The organic mobile phase is 0.1% formic acid in acetonitrile. Flow rate is maintained at 0.8 mL/minute using Shimadzu pumps. The column is ACE 5 Phenyl, 50×2.1 mm. The analysis was performed on a SCIEX triple quadrupole mass spectrometer operating in positive ion mode. The effluent from the HPLC column was directly introduced into the electrospray ionization (ESI). Multiple reaction monitoring (MRM) is used to determine analyte and internal standard (IS) responses. The MRM for adenosine is 268.1/136.1, for 13C5-Adenosine (IS) is 273.2/136.2. The data is calculated using the peak area ratio (PAR) semi-quantitative method.

TABLE 3
Enzymatic IC₅₀ values of the compounds
of this invention against CD73

| Example No | IC₅₀ (nM) |
|---|---|
| 2 | 0.89 |
| 5 | 0.96 |
| 7 | 1.94 |
| 8 | 3.88 |
| 21 | 0.65 |
| 26 | 0.33 |
| 27 | 0.15 |
| 28 | 0.08 |
| 29A | 0.38 |
| 30A | 0.15 |
| 31A | 0.10 |
| 32A | 0.15 |
| 33A | 0.13 |
| 36 | 2.23 |
| 38 | 0.25 |
| 39 | 0.7 |
| 46 | 2.0 |

### Example 50: T Cell Proliferation Assay

The purpose of this assay is to characterize the potency of inhibitors of CD73 in rescuing adenosine-mediated inhibition of T cell proliferation. CD4+ or CD8+ T cells were isolated from peripheral blood mononuclear cells (PBMCs, HemaCare) by immunomagnetic negative selection using EasySep™ Isolation Kit (STEMCELL Technologies) following the supplier's protocol. CD4+ or CD8+ T cells were pelleted by centrifugation at 300 gravitational force (g) for 5 minutes at room temperature and re-suspended in PBS. CellTrace™ Violet staining solution (Invitrogen) was added at 1:5,000 and incubated at 37° C. for 20 minutes, protected from light. Complete culture medium [RPMI-1640 (Gibco), 10% Fetal Bovine Serum (Gibco), 2 mM GlutaMAX (Gibco) and 1 mM Sodium Pyruvate (Gibco), 100 U/mL Penicillin-Streptomycin (Gibco) and MEM non-essential amino acids (NEAA) cell culture supplement (1:100, Gibco)] was then added, mixed, and incubated at 37° C. for 5 minutes. Cells were then pelleted by centrifugation at 300 g for 5 minutes at room temperature and re-suspended in fresh, pre-warmed complete culture medium. 50 μL of cells were seeded per well in 96 well u-bottom plates. 50 μL of CD3/CD28 beads-containing medium and 50 μL of media containing compounds were added into cells. 50 μL of media containing AMP and EHNA hydrochloride (Sigma-Aldrich) was added into cells at 200 μM and 5 μM final concentration, respectively. Cells were incubated for 72 hours at 37° C. in a 5% CO₂ incubator. 200 μL of PBS was then added to each well and cells were centrifuged at 300 g, 4° C. for 10 minutes. The supernatant was discarded. 50 μL of Human TruStain FcX™ (Fc Receptor Blocking Solution, BioLegend) diluted 1:100 in PBS was added to each well, mixed gently and incubated for 20 minutes at 4° C. 50 μL of staining solution (BioLegend) was added to each well, mixed gently and incubated at 4° C. for 30 minutes. Cells were centrifuged at 300 g, 4° C. for 10 minutes and the supernatant was discarded. The cell pellets were washed with 250 μL of cell staining buffer and centrifuged at 300 g, 4° C. for 10 minutes. The supernatant was discarded and cells were re-suspended in 60 μL of cell staining buffer and analyzed on a flow cytometer.

TABLE 4
Rescue of proliferation of CD4+ and CD8+ T cells
under high AMP condition by CD73 inhibitors

| | CD8+ T cell proliferation | CD4+ T cell proliferation |
|---|---|---|
| Example No | EC₅₀ (nM) | EC₅₀ (nM) |
| 26 | 3.9 | 5.6 |
| 27 | 3.84 | 4.3 |
| 28 | 0.81 | 1.08 |
| 31A | 1.38 | 2.1 |
| 32A | 2.05 | 3.88 |
| 33A | 0.63 | 0.84 |

### Example 51: T Cell Cytokine Release Function Assay

The purpose of this assay is to characterize the potency of inhibitors of CD73 in rescuing adenosine-mediated inhibition of T cell cytokine release function. CD4+ or CD8+ T cells were isolated from peripheral blood mononuclear cells (PBMCs) by immunomagnetic negative selection using EasySep™ Isolation Kit (STEMCELL Technologies) following the supplier's protocol. CD4+ or CD8+ T cells were then pelleted the cells by centrifugation at 300 g for 5 minutes at room temperature and re-suspended in fresh, pre-warmed complete culture medium. 50 μL of cells was seeded per well in 96 well u-bottom plates. 50 μL of CD3/CD28 beads-containing medium and 50 μL of media containing compounds were added into cells. 50 μL of media containing AMP and EHNA hydrochloride (Sigma-Aldrich) was added into cells at 200 μM and 5 μM final concentration, respectively. Cells were incubated for up to 72 hours at 37° C. in a 5% CO₂ incubator. 50 μL of supernatant was collected to determine levels of IL2 and IFN gamma using ELISA-MSD kit (Meso Scale Discovery).

1. A compound of formula (I), 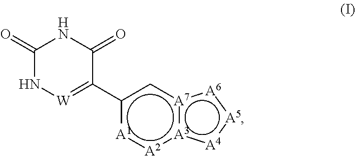
wherein
W is CH or N;
A¹ and A² are each independently CH or N;
A³ and A⁷ are each independently C or N;
A⁴, A⁵ and A⁶ are each independently O, S, N, CR¹ or NR²;
R¹ is H, halogen, cyano, C₁₋₆alkyl, C₃₋₇cycloalkyl, C₁₋₆alkoxyC₁₋₆alkyl or -L¹-R³;
R² is H, C₁₋₆alkyl, C₃₋₇cycloalkyl, C₁₋₆alkoxyC₁₋₆alkyl or -L²-R³;
wherein L¹ is O, S, NH, NR³, C₁₋₆alkylene, C₃₋₇cycloalkylene, heteroarylene or heterocyclylene;
L² is C₁₋₆alkylene, C₃₋₇cycloalkylene, heteroarylene or heterocyclylene;
R³ is optionally substituted group selected from C₁₋₆alkyl, C₃₋₇cycloal, C₃₋₇cycloalC₁₋₆alkyl, C₁₋₆alkoxyC₁₋₆alkyl, aryl, heteroaryl, heterocyclyl, arylC₁₋₆alkyl, heterocyclylC₁₋₆alkyl, heteroarylC₁₋₆alkyl, arylhaloC₁₋₆alkyl, heterocyclylhaloC₁₋₆alkyl and heteroarylhaloC₁₋₆alkyl;
or a pharmaceutically acceptable salt thereof.

2. A compound according to claim 1 having the structure of formula (Ia): 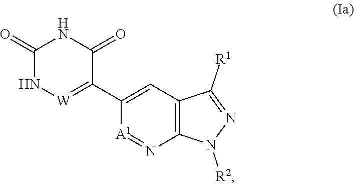
wherein
W is CH;
A¹ is N;
R¹ is (C₁₋₆alkyl)₂amino, (C₁₋₆alkylhalopyrazolyl)C₁₋₆alkoxy, (C₁₋₆alkylhalopyridinyl)C₁₋₆alkoxy, (C₁₋₆alkylpyrazolyl)C₁₋₆alkoxy, (C₁₋₆alkylpyridinyl)C₁₋₆alkoxy, (C₁₋₆alkylpyridinyl)haloC₁₋₆alkoxy, (C₁₋₆alkylthiazolyl)C₁₋₆alkoxy, (cyanophenyl)C₁₋₆alkoxy, (haloC₁₋₆alkylphenyl)C₁₋₆alkoxy, (halophenyl)C₁₋₆alkoxy, (halopyridazinyl)C₁₋₆alkoxy, (halopyridinyl)C₁₋₆alkoxy, (halopyridinyl)haloC₁₋₆alkoxy, (phenylC₁₋₆alkyl)pyrazolyl, benzoxazolylC₁₋₆alkoxy, C₁₋₆alkoxy, C₁₋₆alkyl, C₃₋₇cycloalkyl, C₃₋₇cycloalkyl(C₁₋₆alkyl)amino, C₃₋₇cycloalkylC₁₋₆alkyl(C₁₋₆alkyl)amino, phenylC₁₋₆alkoxy, phenylC₁₋₆alkyl, phenylC₁₋₆alkyl(C₁₋₆alkyl)amino, phenylC₁₋₆alkylamino, phenylC₃₋₇cycloalkyl, phenylhaloC₁₋₆alkoxy, pyridinylC₁₋₆alkoxy or pyridinylhaloC₁₋₆alkoxy;
R² is C₁₋₆alkyl;
or a pharmaceutically acceptable salt thereof.

3. A compound according to claim 1 or 2, wherein R¹ is (C₁₋₆alkylpyridinyl)haloC₁₋₆alkoxy, (halopyridinyl)haloC₁₋₆alkoxy, (phenylC₁₋₆alkyl)pyrazolyl, C₃₋₇cycloalkyl, C₃₋₇cycloalkylC₁₋₆alkyl(C₁₋₆alkyl)amino, phenylC₁₋₆alkoxy, phenylC₁₋₆alkyl, phenylC₁₋₆alkyl(C₁₋₆alkyl)amino, phenylC₁₋₆alkylamino, phenylC₃₋₇cycloalkyl, phenylhaloC₁₋₆alkoxy, pyridinylC₁₋₆alkoxy or pyridinylhaloC₁₋₆alkoxy.

4. A compound according to any one of claims 1-3, wherein R¹ is (1-phenylethyl)amino, 1-(2-pyridinyl)ethoxy, 1-(2-pyridinyl)ethoxy, 1-cyclopentylethyl(methyl)amino, 1-phenylcyclopropyl, 1-phenylethoxy, 1-phenylethoxy, 1-phenylethyl, 2,2,2-trifluoro-1-(2-pyridinyl)ethoxy, 2,2,2-trifluoro-1-phenyl-ethoxy, 2,2-difluoro-1-(2-pyridinyl)ethoxy, 2,2-difluoro-1-(5-fluoro-2-pyridinyl)ethoxy, 2,2-difluoro-1-(6-methyl-2-pyridinyl)ethoxy, 2,2-difluoro-1-phenyl-ethoxy, 2-benzylpyrazol-3-yl, cyclobutyl or methyl(1-phenylethyl)amino.

5. A compound according to any one of claims 1-4, wherein R¹ is (C₁₋₆alkylpyridinyl)haloC₁₋₆alkoxy, (halopyridinyl)haloC₁₋₆alkoxy, C₃₋₇cycloalkylC₁₋₆alkyl(C₁₋₆alkyl)amino, phenylC₁₋₆alkoxy, phenylC₁₋₆alkyl(C₁₋₆alkyl)amino, phenylC₁₋₆alkyl, phenylC₁₋₆alkylamino, phenylhaloC₁₋₆alkoxy, pyridinylC₁₋₆alkoxy or pyridinylhaloC₁₋₆alkoxy.

6. A compound according to any one of claims 1-4, wherein R¹ is (1-phenylethyl)amino, 1-(2-pyridinyl)ethoxy, 1-cyclopentylethyl(methyl)amino, 1-phenylethoxy, 1-phenylethyl, 2,2-difluoro-1-(2-pyridinyl)ethoxy, 2,2-difluoro-1-(5-fluoro-2-pyridinyl)ethoxy, 2,2-difluoro-1-(6-methyl-2-pyridinyl)ethoxy, 2,2-difluoro-1-phenyl-ethoxy or methyl(1-phenylethyl)amino.

7. A compound according to any one of claims 1-6, wherein R² is methyl.

8. A compound according to any one of claims 2-7, wherein
W is CH;
A¹ is N;
R¹ is (C₁₋₆alkylpyridinyl)haloC₁₋₆alkoxy, (halopyridinyl)haloC₁₋₆alkoxy, C₃₋₇cycloalkylC₁₋₆alkyl(C₁₋₆alkyl)amino, phenylC₁₋₆alkoxy, phenylC₁₋₆alkyl(C₁₋₆alkyl)amino, phenylC₁₋₆alkyl, phenylC₁₋₆alkylamino, phenylhaloC₁₋₆alkoxy, pyridinylC₁₋₆alkoxy or pyridinylhaloC₁₋₆alkoxy;
R² is C₁₋₆alkyl;
or a pharmaceutically acceptable salt thereof.

9. A compound according to any one of claims 2-8, wherein
W is CH;
A¹ is N;
R¹ is (1-phenylethyl)amino, 1-(2-pyridinyl)ethoxy, 1-cyclopentylethyl(methyl)amino, 1-phenylethoxy, 1-phenylethyl, 2,2-difluoro-1-(2-pyridinyl)ethoxy, 2,2-difluoro-1-(5-fluoro-2-pyridinyl)ethoxy, 2,2-difluoro-1-(6-methyl-2-pyridinyl)ethoxy, 2,2-difluoro-1-phenyl-ethoxy or methyl(1-phenylethyl)amino;
R² is methyl;
or a pharmaceutically acceptable salt thereof.

10. A compound according to claim 1 having the structure of formula (Ib): 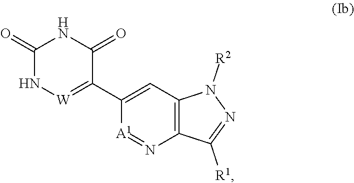
wherein
W is CH;
A¹ is N;
R¹ is H or halogen;
R² is C₁₋₆alkyl;
or a pharmaceutically acceptable salt thereof.

11. A compound according to claim 10, wherein R¹ is halogen.

12. A compound according to claim 10 or 11, wherein R¹ is chloro.

13. A compound according to any one of claims 10-12, wherein R² is methyl.

14. A compound according to any one of claims 10-13, wherein
W is CH;
A¹ is N;
R¹ is chloro;
R² is methyl;
or a pharmaceutically acceptable salt thereof.

15. A compound according to claim 1 having the structure of formula (Ic): 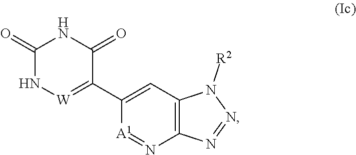
wherein
W is CH;
A¹ is N;
R² is C₁₋₆alkyl;
or a pharmaceutically acceptable salt thereof.

16. A compound according to claim 15, wherein R² is methyl.

17. A compound according to claim 1 having the structure of formula (Id): 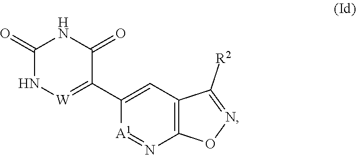
wherein
W is CH;
A¹ is N;
R¹ is C₃₋₇cycloalkyl;
or a pharmaceutically acceptable salt thereof.

18. A compound according to claim 17, wherein R¹ is cyclobutyl.

19. A compound selected from:
5-(1,3-dimethylpyrazolo[3,4-c]pyridazin-5-yl)-1H-pyrimidine-2,4-dione;
5-(3-cyclopropyl-1-methyl-pyrazolo[3,4-c]pyridazin-5-yl)-1H-pyrimidine-2,4-dione;
5-(1-methylpyrazolo[4,3-c]pyridazin-6-yl)-1H-pyrimidine-2,4-dione;
5-(1-methyltriazolo[4,5-c]pyridazin-6-yl)-1H-pyrimidine-2,4-dione;
5-[1-methyl-3-(1-phenylethyl)pyrazolo[3,4-c]pyridazin-5-yl]-1H-pyrimidine-2,4-dione;
5-[1-methyl-3-(1-phenylcyclopropyl)pyrazolo[3,4-c]pyridazin-5-yl]-1H-pyrimidine-2,4-dione;
5-[3-(2-benzylpyrazol-3-yl)-1-methyl-pyrazolo[3,4-c]pyridazin-5-yl]-1H-pyrimidine-2,4-dione;
5-(3-isopropoxy-1-methyl-pyrazolo[3,4-c]pyridazin-5-yl)-1H-pyrimidine-2,4-dione;
5-(3-chloro-1-methyl-pyrazolo[4,3-c]pyridazin-6-yl)-1H-pyrimidine-2,4-dione;
5-[1-methyl-3-(1-phenylethoxy)pyrazolo[3,4-c]pyridazin-5-yl]-1H-pyrimidine-2,4-dione;
3-[1-[5-(2,4-dioxo-1H-pyrimidin-5-yl)-1-methyl-pyrazolo[3,4-c]pyridazin-3-yl]oxyethyl]benzonitrile;
4-[1-[5-(2,4-dioxo-1H-pyrimidin-5-yl)-1-methyl-pyrazolo[3,4-c]pyridazin-3-yl]oxyethyl]benzonitrile;
5-[3-[1-(2-chlorophenyl)ethoxy]-1-methyl-pyrazolo[3,4-c]pyridazin-5-yl]-1H-pyrimidine-2,4-dione;
5-[3-[1-(3-chlorophenyl)ethoxy]-1-methyl-pyrazolo[3,4-c]pyridazin-5-yl]-1H-pyrimidine-2,4-dione;
5-[3-[1-(4-chlorophenyl)ethoxy]-1-methyl-pyrazolo[3,4-c]pyridazin-5-yl]-1H-pyrimidine-2,4-dione;
5-[3-[1-(4-fluorophenyl)ethoxy]-1-methyl-pyrazolo[3,4-c]pyridazin-5-yl]-1H-pyrimidine-2,4-dione;
5-[1-methyl-3-[1-(2-pyridyl)ethoxy]pyrazolo[3,4-c]pyridazin-5-yl]-1H-pyrimidine-2,4-dione;
5-[1-methyl-3-[1-(3-pyridyl)ethoxy]pyrazolo[3,4-c]pyridazin-5-yl]-1H-pyrimidine-2,4-dione;
5-[1-methyl-3-[1-(4-pyridyl)ethoxy]pyrazolo[3,4-c]pyridazin-5-yl]-1H-pyrimidine-2,4-dione;
5-[1-methyl-3-[1-[3-(trifluoromethyl)phenyl]ethoxy]pyrazolo[3,4-c]pyridazin-5-yl]-1H-pyrimidine-2,4-dione;
5-[1-methyl-3-[1-(2-methylthiazol-4-yl)ethoxy]pyrazolo[3,4-c]pyridazin-5-yl]-1H-pyrimidine-2,4-dione;
5-[1-methyl-3-[1-(5-methylthiazol-2-yl)ethoxy]pyrazolo[3,4-c]pyridazin-5-yl]-1H-pyrimidine-2,4-dione;
5-[3-[1-(4-chloro-1-methyl-pyrazol-3-yl)ethoxy]-1-methyl-pyrazolo[3,4-c]pyridazin-5-yl]-1H-pyrimidine-2,4-dione;
5-[1-methyl-3-[1-(2-methylpyrazol-3-yl)ethoxy]pyrazolo[3,4-c]pyridazin-5-yl]-1H-pyrimidine-2,4-dione;
5-[3-[1-(1,3-benzoxazol-2-yl)ethoxy]-1-methyl-pyrazolo[3,4-c]pyridazin-5-yl]-1H-pyrimidine-2,4-dione;
5-[1-methyl-3-[(1S)-1-(2-pyridyl)ethoxy]pyrazolo[3,4-c]pyridazin-5-yl]-1H-pyrimidine-2,4-dione;
5-[1-methyl-3-[(1S)-1-phenylethoxy]pyrazolo[3,4-c]pyridazin-5-yl]-1H-pyrimidine-2,4-dione;
5-[3-[(1R)-2,2-difluoro-1-phenyl-ethoxy]-1-methyl-pyrazolo[3,4-c]pyridazin-5-yl]-1H-pyrimidine-2,4-dione;
5-[1-methyl-3-[(1R)-2,2,2-trifluoro-1-(2-pyridyl)ethoxy]pyrazolo[3,4-c]pyridazin-5-yl]-1H-pyrimidine-2,4-dione;
5-[1-methyl-3-[(1S)-2,2,2-trifluoro-1-(2-pyridyl)ethoxy]pyrazolo[3,4-c]pyridazin-5-yl]-1H-pyrimidine-2,4-dione;
5-[1-methyl-3-[(1R)-2,2,2-trifluoro-1-phenyl-ethoxy]pyrazolo[3,4-c]pyridazin-5-yl]-1H-pyrimidine-2,4-dione;
5-[1-methyl-3-[(1S)-2,2,2-trifluoro-1-phenyl-ethoxy]pyrazolo[3,4-c]pyridazin-5-yl]-1H-pyrimidine-2,4-dione;
5-[3-[(1R)-2,2-difluoro-1-(2-pyridyl)ethoxy]-1-methyl-pyrazolo[3,4-c]pyridazin-5-yl]-1H-pyrimidine-2,4-dione;
5-[3-[(1S)-2,2-difluoro-1-(2-pyridyl)ethoxy]-1-methyl-pyrazolo[3,4-c]pyridazin-5-yl]-1H-pyrimidine-2,4-dione;
5-[3-[(1R)-2,2-difluoro-1-(5-fluoro-2-pyridyl)ethoxy]-1-methyl-pyrazolo[3,4-c]pyridazin-5-yl]-1H-pyrimidine-2,4-dione;
5-[3-[(1S)-2,2-difluoro-1-(5-fluoro-2-pyridyl)ethoxy]-1-methyl-pyrazolo[3,4-c]pyridazin-5-yl]-1H-pyrimidine-2,4-dione;
5-[3-[(1R)-2,2-difluoro-1-(6-methyl-2-pyridyl)ethoxy]-1-methyl-pyrazolo[3,4-c]pyridazin-5-yl]-1H-pyrimidine-2,4-dione;
5-[3-[(1S)-2,2-difluoro-1-(6-methyl-2-pyridyl)ethoxy]-1-methyl-pyrazolo[3,4-c]pyridazin-5-yl]-1H-pyrimidine-2,4-dione;
5-[1-methyl-3-[methyl(1,2,2-trimethylpropyl)amino]pyrazolo[3,4-c]pyridazin-5-yl]-1H-pyrimidine-2,4-dione;
5-[3-[1-cyclopentylethyl(methyl)amino]-1-methyl-pyrazolo[3,4-c]pyridazin-5-yl]-1H-pyrimidine-2,4-dione;
5-[3-[cyclopentyl(methyl)amino]-1-methyl-pyrazolo[3,4-c]pyridazin-5-yl]-1H-pyrimidine-2,4-dione;
5-[1-methyl-3-[[(1S)-1-phenylethyl]amino]pyrazolo[3,4-c]pyridazin-5-yl]-1H-pyrimidine-2,4-dione;
5-[1-methyl-3-[methyl-[(1S)-1-phenylethyl]amino]pyrazolo[3,4-c]pyridazin-5-yl]-1H-pyrimidine-2,4-dione;
5-(3-cyclobutyl-1-methyl-pyrazolo[3,4-c]pyridazin-5-yl)-1H-pyrimidine-2,4-dione;
5-[3-[(1S)-1-(5-fluoro-6-methyl-2-pyridyl)ethoxy]-1-methyl-pyrazolo[3,4-c]pyridazin-5-yl]-1H-pyrimidine-2,4-dione;
5-[1-methyl-3-[(1S)-1-(6-methyl-2-pyridyl)ethoxy]pyrazolo[3,4-c]pyridazin-5-yl]-1H-pyrimidine-2,4-dione;
5-[1-methyl-3-[(1S)-1-(4-methyl-2-pyridyl)ethoxy]pyrazolo[3,4-c]pyridazin-5-yl]-1H-pyrimidine-2,4-dione;
5-[3-[(1S)-1-(5-fluoro-2-pyridyl)ethoxy]-1-methyl-pyrazolo[3,4-c]pyridazin-5-yl]-1H-pyrimidine-2,4-dione;
5-[3-[(1S)-1-(6-chloro-2-pyridyl)ethoxy]-1-methyl-pyrazolo[3,4-c]pyridazin-5-yl]-1H-pyrimidine-2,4-dione;
5-[3-[(1S)-1-(6-chloropyridazin-3-yl)ethoxy]-1-methyl-pyrazolo[3,4-c]pyridazin-5-yl]-1H-pyrimidine-2,4-dione; and
5-(3-cyclobutylisoxazolo[5,4-c]pyridazin-5-yl)-1H-pyrimidine-2,4-dione;
or a pharmaceutically acceptable salt thereof.

20. A process for the preparation of a compound according to any one of claims 1 to 19 comprising any of the following steps:
a) Deprotection of compound of formula (XVI), 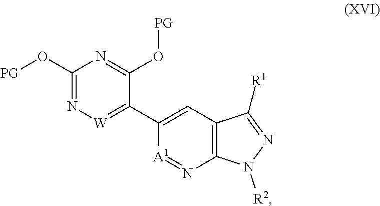
with an acid or dealkylation reagent, or through hydrogenation to afford the compound of formula (Ia), 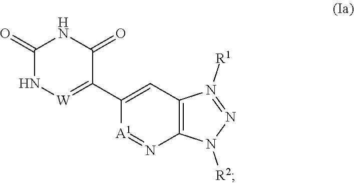
b) Deprotection of compound of formula (XXI), 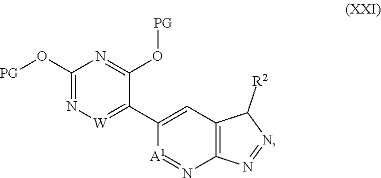
with an acid or dealkylation reagent, or through hydrogenation to afford the compound of formula (Ib-1), 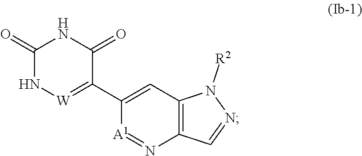
c) Deprotection of compound of formula (XXVI), 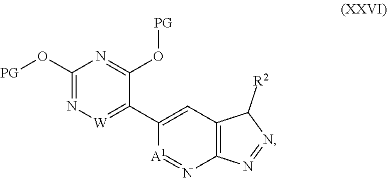
with an acid or dealkylation reagent, or through hydrogenation to afford the compound of formula (Ic), 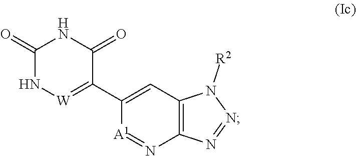
d) Deprotection of compound of formula (XXXII), 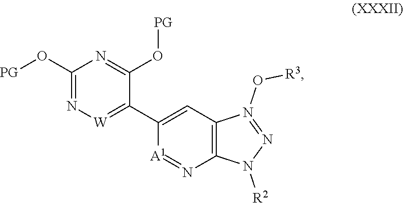
with an acid or dealkylation reagent, or through hydrogenation to afford the compound of formula (Ia-1), 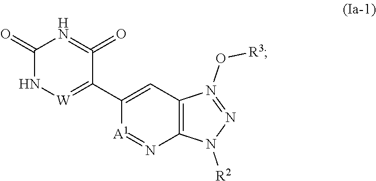
e) Deprotection of compound of formula (XXXVI), 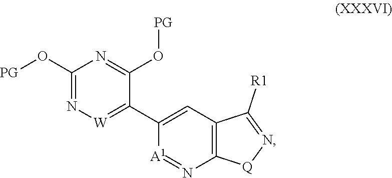
with an acid or dealkylation reagent, or through hydrogenation to afford the compound of formula (XXXVII), 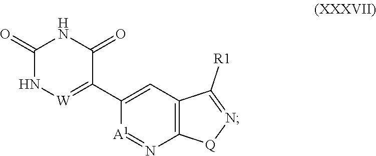
wherein
each PG is independently an oxygen protecting group; wherein PG is selected from methyl, tert-butyl, TBS, ethoxymethyl and benzyl;
in step a), b), c), d) and e), the acid is trifluoroacetic acid or aqueous hydrochloric acid;
the dealkylation reagent is TMSCl and NaI; the hydrogenation is conducted with Pd/C;
A¹, W, R¹ to R³ are defined as in any one of claims 1 to 18.

21. A compound or pharmaceutically acceptable salt according to any one of claims 1 to 19 for use as therapeutically active substance.

22. A pharmaceutical composition comprising a compound in accordance with any one of claims 1 to 19 and a pharmaceutically acceptable excipient.

23. The use of a compound according to any one of claims 1 to 19 for treating cancers.

24. The use according to claim 23, wherein the cancer is pancreatic cancer, colorectal cancer, gastric cancer, esophageal cancer, liver cancer, lung cancer, breast cancer, ovarian cancer, prostate cancer or melanoma.

25. The use of a compound according to any one of claims 1 to 19 for inhibiting CD73.

26. The use of a compound according to any one of claims 1 to 19 for the preparation of a medicament for the treatment or prophylaxis of cancers, wherein the cancer is pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia.

27. The use of a compound according to any one of claims 1 to 19 for the preparation of a medicament as a CD73 inhibitor.

28. A compound or pharmaceutically acceptable salt according to any one of claims 1 to 19, when manufactured according to a process of claim 20.

29. The invention as hereinbefore described.
